(19) United States
(12) Patent Application Publication
(10) Pub. No.: US 2021/0130401 A1
(43) Pub. Date: May 6, 2021

(54) PEPTIDOMIMETIC INHIBITORS OF THE WDR5-MLL INTERACTION

(71) Applicant: THE REGENTS OF THE UNIVERSITY OF MICHIGAN, Ann Arbor, MI (US)

(72) Inventors: Shaomeng Wang, Superior Township, MI (US); Yangbing Li, Ann Arbor, MI (US); Hacer Karatas, Ann Arbor, MI (US); Liu Liu, Ann Arbor, MI (US); Jeanne Stuckey, Fenton, MI (US); Yali Dou, Ann Arbor, MI (US); Liyue Huang, Ann Arbor, MI (US); Atsunori Kaneshige, Ann Arbor, MI (US)

(21) Appl. No.: 16/605,057

(22) PCT Filed: May 15, 2018
(86) PCT No.: PCT/US18/32646

§ 371 Date: Oct. 14, 2019

Related U.S. Application Data
(60) Provisional application No. 62/506,277, filed on May 15, 2017.

(51) Int. Cl. C07K 5/11; A61K 38/07; A61K 45/06

(52) U.S. Cl. CPC C07K 5/1019; A61K 45/06; A61K 38/07

(57) ABSTRACT

The present disclosure provides compounds represented by Formula I: and the pharmaceutically acceptable salts and solvates thereof, wherein R¹, R², R^{3a}, R^{3b}, R^{4a}, R^{4b}, R^{5a} and R^{5b} are as defined as set forth in the specification. The present disclosure also provides compounds of Formula I for use to treat a condition, disease, or disorder responsive to inhibition of the WDR5 interaction with its binding partners including, but not limited to, the WDR5-MLL protein-protein interaction.

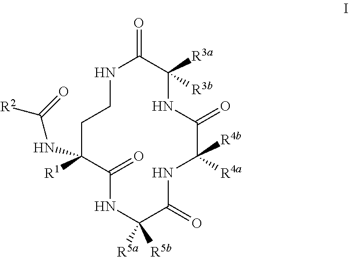

This invention was made with government support under grant number CA177307 awarded by the National Institutes of Health and under Contract No. DE AC02-06CH11357 awarded by the U.S. Department of Energy. The government has certain rights in this invention.

### BACKGROUND OF THE INVENTION

### Field of Invention

The present disclosure provides cyclic peptidomimetics that bind to WD repeat domain 5 protein (WDR5) and block the interactions between WDR5 and its binding partner proteins including, but not limited to, mixed lineage leukemia (MLL) protein. The present disclosure also provides therapeutic methods of treating conditions and diseases, e.g., cancer, wherein inhibition of the WDR5-binding partner protein-protein interaction provides a benefit.

### Background

Histones are important for the organization of DNA into a chromatin structure and in the retrieval of genetic information. Specific modifications on histones regulate gene activity, leading to either expression or silence (Kouzarides, Cell 128:693-705 (2007); Jenuwein and Allis, Science 293:1074-1080 (2001)). Of the modifications in the euchromatins of eukaryotes that have been examined, Histone 3-Lysine 4 (H3-K4) trimethylation is recognized as a hallmark of transcriptionally active genes (Shilatifard, Curr Opin Cell Biol 20:341-348 (2008)). It is believed that trimethylated H3-K4 is a recognition site for the recruitment of additional factors required for transcription (Sims and Reinberg, Genes Dev 20:2779-2786 (2006); Wysocka et al., Nature 442:86-90 (2006)). Abnormalities in H3-K4 methylating enzymes have been observed in various cancers (Huntsman et al., Oncogene 18:7975-7984 (1999); Ruault et al., Gene 284:73-81 (2002)), the most prominent example of which is Mixed Lineage Leukemia (MLL) (Hess, Trends Mol Med 10:500-507 (2004)), which is also known as MLL1, ALL-1, HRX, and HTRX1.

MLL is enzymatically active in a multiprotein complex and acts as both a global and a specific gene regulator (Guenther et al., Proc Natl Acad Sci USA 102:8603-8608 (2005); Mishra et al., FEBS J 276:1629-1640 (2009)). The most well-known targets for MLL are the homeobox (Hox) genes such as Hox-a9 and Hox-c8. These genes encode for a class of homeodomain transcriptional factors that regulate organ formation during embryo development, as well as proper hematopoiesis in adults (Hombria and Lovegrove, Differentiation 71:461-476 (2003); Monier et al., Fly (Austin) 1:59-67 (2007); Jude et al., Cell Stem Cell 1:324-337 (2007)). Increased expression levels of certain Hox genes, accompanied by MLL aberrations, such as gene fusion and amplification, are frequently observed in acute leukemias, such as acute lymphoblastic leukemia (ALL) and acute myeloid leukemia (AML) (Ferrando et al., Blood 102:262-268 (2003); Harper and Aplan, Cancer Res 68:10024-10027 (2008); Argiropoulos and Humphries, Oncogene 26:6766-6776 (2007)). Injection of cells overexpressing Hox-a7 and Hox-c8 into nude mice results in well vascularized tumors in 4-5 weeks (Maulbecker and Gruss, Cell Growth Differ 4:431-441 (1993)). Abnormal Hox gene expression also is observed in solid tumors, such as prostate carcinoma and primary colorectal tumors (Waltregny et al., Prostate 50:162-169 (2002); De Vita et al., Eur J Cancer 29A:887-893 (1993)). MLL therefore is a promising therapeutic target for several forms of leukemias and solid tumors.

Immediately after translation, MLL is proteolytically cleaved to yield 180-kDa C-terminus (MLL1c) and 320-kDa N-terminus fragments (MLL1^{N}) (Hsieh et al., Mol Cell Biol 23:186-194 (2003)). These are assembled together in a multi-subunit complex together with several other proteins, including WD Repeat Domain 5 (WDR5), Absent Small or Homeotic-Like (Ash2L), and Retinoblastoma Binding Protein 5 (RbBP5), each of which is a common component of all known human H3-K4 methylating complexes.

MLL forms a catalytically active core complex with WDR5, RbBP5, and Ash2L that can dimethylate H3-K4 in vitro (Patel et al., J Biol Chem 283:32162-32175 (2008)). Although MLL alone can minimally partially monomethylate H3-K4, all the other members of the core complex are required for dimethylation, including WDR5, which forms a bridge between MLL and the remainder of the core complex. In the absence of WDR5, MLL is unable to associate with RbBP5 and Ash2L, and fails to dimethylate H3-K4 in vitro (Patel et al., J Biol Chem 283:32162-32175 (2008); Dou et al., Nat Struct Mol Biol 13:713-719 (2006)). Knock-down of WDR5 is known to result in a significant decrease in the levels of H3-K4 trimethylation and expression of Hox-a9 and Hox-c8 genes in 293 cells (Wysocka et al., Cell 121:859-872 (2005)). Blocking of the WDR5-MLL interaction therefore is an effective strategy for inhibiting MLL activity.

It has been shown that MLL binds to WDR5 via an arginine (Arg) (residue 3765) containing sequence (Song et al., J Biol Chem 283:35258-35264 (2008); Patel et al., J Biol Chem 283:32158-32161 (2008)), which is similar to that used by the N-terminal of H3 in its interaction with WDR5 (Schuetz et al., EMBO J 25:4245-4252 (2006); Han et al., Mol Cell 22:137-144 (2006); Couture et al., Nat Struct Mol Biol 13:698-703 (2006); Ruthenburg et al., Nat Struct Mol Biol 13:704-712 (2006)). WDR5 has a canonical conformation that contains a central cavity, and both H3 and MLL peptides use an Arg residue to interact with this cavity through the arginine binding site. Although crystal structures show that H3 and MLL peptides have very similar binding modes to WDR5 in this arginine binding site, MLL peptides have a higher affinity to WDR5 than H3 peptides (Trievel and Shilatifard, Nat Struct Mol Biol 16:678-680 (2009)). The MLL-derived, 12-residue WIN (WDR5 Interacting Motif) peptide (residues 3762-3773) has been shown to dissociate MLL from the remainder of the complex in vitro (Patel et al., J Biol Chem 283:32162-32175 (2008)).

Compounds that block the interaction of MLL with WDR5 are being developed for the treatment of cancer and other diseases. See, e.g., Senisterra et al., Biochem. J. 449:151-159 (2013); Getlik et al., J. Med. Chem. 59:2478-96 (2016); Li et al., Eur. J. Med. Chem. 124:480-489 (2016); Li et al., Eur. J. Med. Chem. 118:1-8 (2016); Li et al., Bioorg. Med. Chem. Lett. 24:6109-6118 (2016); Grebien et al., Nat. Chem. Biol. 11:571-578 (2015); Karatas et al., J. Am. Chem. Soc. 135:669-682 (2013); Cao et al., Mol Cell 53:247-61 (2014); Karatas et al., J. Med. Chem. 53:5179-5185 (2010); U.S. Pat. Nos. 9,233,086; and 8,980,838. There is a need in the art for new inhibitors of the WDR5-MLL protein-protein interaction.

### BRIEF SUMMARY OF THE INVENTION

In one aspect, the present disclosure provides compounds represented by any one of Formulae I-III, below, and the pharmaceutically acceptable salts and solvates, e.g., hydrates, thereof, collectively referred to as “Compounds of the Disclosure.” Compounds of the Disclosure are inhibitors of the WDR5-binding partner protein-protein interaction, e.g., the WDR5-MLL protein-protein interaction, or synthetic intermediates used to prepare inhibitors of the WDR5-binding partner protein-protein interaction. Inhibitors of the WDR5-binding partner protein-protein interaction are useful in treating or preventing diseases or conditions such as cancer.

In another aspect, the present disclosure provides methods of treating or preventing a condition or disease by administering a therapeutically effective amount of a Compound of the Disclosure to a subject, e.g., a human patient, in need thereof. The disease or condition of interest treatable or preventable by inhibition the WDR5-binding partner protein-protein interaction, e.g., the WDR5-MLL protein-protein interaction, is, for example, a cancer, a chronic autoimmune disorder, an inflammatory condition, a proliferative disorder, sepsis, or a viral infection. Also provided are methods of preventing the proliferation of unwanted proliferating cells, such as in cancer, in a subject comprising administering a therapeutically effective amount of a Compound of the Disclosure to a subject at risk of developing a condition characterized by unwanted proliferating cells. In some embodiments, the Compounds of the Disclosure may reduce the proliferation of unwanted cells by inducing apoptosis in those cells.

In another aspect, the present disclosure provides a method of inhibiting the WDR5-binding partner protein-protein interaction, e.g., the WDR5-MLL protein-protein interaction, in a subject, comprising administering to the subject a therapeutically effective amount of at least one Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating or preventing diseases or conditions wherein inhibition of the WDR5-binding partner protein-protein interaction, e.g., WDR5-MLL protein-protein, provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment or prevention of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF DRAWINGS

FIG. 1 is an illustration showing the assay flow of the homogeneous AlphaLISA MLL HMT functional assay.

FIG. 2 are two line graphs (B) and (C) showing the enzymatic activity of the MLL complex, represented by the fluorescence intensity in the relative fluorescence units (RFU) from acceptor beads detected at 615 nm at different concentrations and different reaction time points with a low (300 nM, B) or a high (3 μM, C) SAM concentration.

FIG. 3 is a line graph showing representative inhibitory curves of selected WDR5 inhibitors.

FIG. 4 is scatter plot showing the comparison of potencies of compounds determined from the competitive WDR5 binding assay and the AlphaLISA MLL functional assay.

FIG. 5 is a line graph showing cell growth inhibition curves of representative cyclic peptidomimetics in MV4;11 human leukemia cell lines harboring MLL translocation.

FIG. 6 is a line graph showing cell growth inhibition curves of representative cyclic peptidomimetics in MOLM-13 human leukemia cell lines harboring MLL translocation.

FIG. 7 is a line graph showing the correlation between inhibition potencies of cell growth and MLL HMT activity of representative cyclic peptidomimetics.

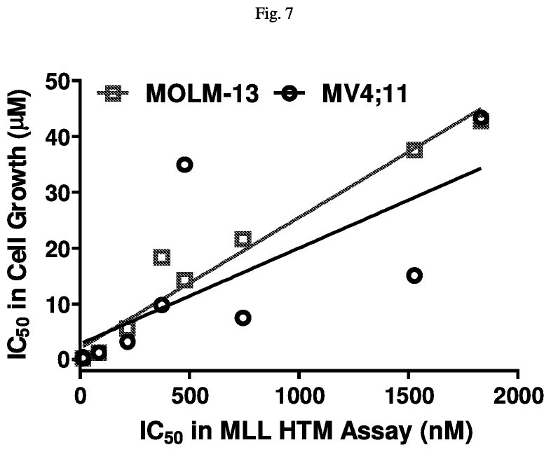

FIG. 8 is a line graph showing cell growth inhibition of Cpd. No. 5 upon 4-day or 7-day treatment time in MV4;11 acute leukemia cell lines.

FIG. 9 is a line graph showing cell growth inhibition of Cpd. No. 5 upon 4-day or 7-day treatment time in MOLM-13 acute leukemia cell lines.

FIG. 10 is a series of four illustrations showing the co-crystal structure of Cpd. No. 76 in complex with WDR5 (A-C) and in comparison with co-crystal structure of Cpd. No. 64 in complex with WDR5 (D).

FIG. 11 is a series of four illustrations showing the co-crystal structure of cyclic peptidomimetic Cpd. No. 5 in complex with WDR5. (A) The unbiased difference electron density map for Cpd. No. 5 contoured at 3σ is shown as a grey grid. (B) Structural alignment of WDR5-Cpd. No. 5 with the co-crystal structures of compounds Cpd. No. 64 and Cpd. No. 76 in complex with WDR5. (C) The hydrogen bonding and (D) hydrophobic interactions of WDR5 and Cpd. No. 5. Dashed lines represent hydrogen bonds in (C).

### DETAILED DESCRIPTION OF THE INVENTION

Compounds of the Disclosure are cyclic peptidomimetics that bind to WDR5 and block the WDR5-binding partner protein-protein interaction, e.g., the WDR5-MLL protein-protein interaction.

In one embodiment, Compounds of the Disclosure are compounds represented by Formula I:

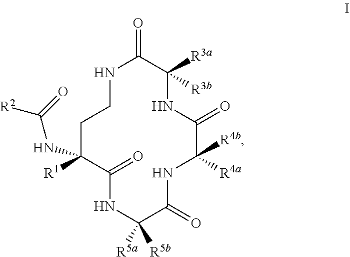

or a pharmaceutically acceptable salt or solvate thereof, wherein:

R¹ is selected from the group consisting of hydrogen, C₁₋₄ alkyl, and C₃₋₈ cycloalkyl;

R² is selected from the group consisting of C₁₋₆ alkyl and C₃₋₈ cycloalkyl;

R^{3a} and R^{3b} are independently selected from the group consisting of hydrogen, C₁₋₆ alkyl, C₃₋₈ cycloalkyl, (cycloalkyl)alkyl, aralkyl, optionally substituted aryl, and optionally substituted heteroaryl; or

R^{3a} and R^{3b} taken together with the carbon atom to which they are attached form a C₃₋₈ cycloalkyl;

R^{4a} and R^{4b} are independently selected from the group consisting of hydrogen and C₁₋₄ alkyl; or

R^{4a} and R^{4b} taken together with the carbon atom to which they are attached form a C₃₋₈ cycloalkyl;

R^{5a} and R^{5b} are independently selected from the group consisting of hydrogen, C₁₋₄ alkyl, and —(R^{6a}R^{6b})ₘX—R⁹;

each R^{6a} and R^{6b} are independently selected from the group consisting of hydrogen and C₁₋₄ alkyl;

m is 1, 2, 3, 4, or 5;

X is selected from the group consisting of —N(R^{6c})—, —C(═O)NR^{6d}, —N(R^{6e})C(═NR⁷)NR⁸—, and —N(R^{6d})C(═O)NR^{8a}—; or

X is absent;

R^{6c} is selected from the group consisting of hydrogen and C₁₋₆ alkyl;

R^{6d} is selected from the group consisting of hydrogen and C₁₋₆ alkyl;

R^{6e} is selected from the group consisting of hydrogen and C₁₋₆ alkyl;

R⁷ is selected from the group consisting of hydrogen, C₁₋₆ alkyl, and C₁₋₆ haloalkyl;

R⁸ is selected from the group consisting of hydrogen and C₁₋₆ alkyl; and

R⁹ is selected from the group consisting of hydrogen, nitro, cyano, amino, C₁₋₆ alkyl, aralkyl, (heteroaryl)alkyl, optionally substituted C₃₋₇ cycloalkyl, optionally substituted 4- to 8-membered heterocyclo, optionally substituted 5- to 10-membered heteroaryl, optionally substituted C₆₋₁₀ aryl, —C(═O)R¹⁰, —C(═NH)R¹¹, and —S(═O)₂R¹;

R¹⁰ is selected from the group consisting of C₁₋₆ alkyl, C₁₋₆ haloalkyl, and C₁₋₆ alkenyl,

R¹¹ is amino; and

R¹² is selected from the group consisting of C₁₋₆ alkyl, C₁₋₆ haloalkyl, and C₁₋₆ alkenyl.

In another embodiment, Compounds of the Disclosure are compounds represented by Formula I, or a pharmaceutically acceptable salt or solvate thereof, wherein R^{3b} is hydrogen. In another embodiment, R^{3b} is methyl.

In another embodiment, Compounds of the Disclosure are compounds represented by Formula I, or a pharmaceutically acceptable salt or solvate thereof, wherein R^{3a} is hydrogen.

In another embodiment, Compounds of the Disclosure are compounds represented by Formula I, or a pharmaceutically acceptable salt or solvate thereof, wherein R^{4b} is hydrogen.

In another embodiment, Compounds of the Disclosure are compounds represented by Formula I, or a pharmaceutically acceptable salt or solvate thereof, wherein R^{4a} is hydrogen.

In another embodiment, R^{4a} and R^{4b} taken together with the carbon atom to which they are attached form a C₃₋₆ cycloalkyl.

In another embodiment, Compounds of the Disclosure are compounds represented by Formula I, or a pharmaceutically acceptable salt or solvate thereof, wherein R^{5b} is hydrogen.

In another embodiment, Compounds of the Disclosure are compounds represented by Formula I, or a pharmaceutically acceptable salt or solvate thereof, wherein R^{5a} is hydrogen.

In another embodiment, Compounds of the Disclosure are compounds represented by Formula II:

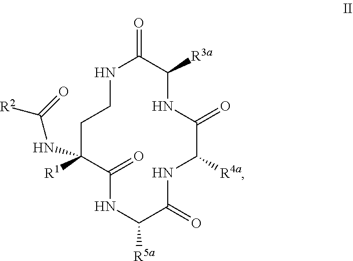

or a pharmaceutically acceptable salt or solvate thereof, wherein:

R¹ is selected from the group consisting of hydrogen and methyl; and

R², R^{3a}, R^{4a}, and R^{5a} are as defined in connection with Formula I.

In another embodiment, Compounds of the Disclosure are compounds represented by Formulae I or II, or a pharmaceutically acceptable salt or solvate thereof, wherein R¹ is methyl.

In another embodiment, Compounds of the Disclosure are compounds represented by Formulae I or II, or a pharmaceutically acceptable salt or solvate thereof, wherein R² is C₁₋₄ alkyl. In another embodiment, R² is —CH(CH₃)₂.

In another embodiment, Compounds of the Disclosure are compounds represented by Formula III:

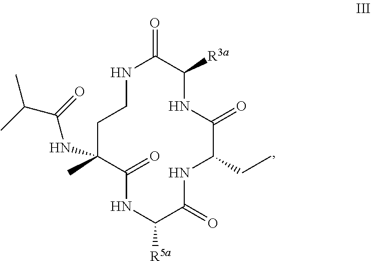

or a pharmaceutically acceptable salt or solvate thereof, wherein R^{3a} and R^{5a} are as defined in connection with Formula I.

In another embodiment, Compounds of the Disclosure are compounds represented by any one of Formulae I-III, or a pharmaceutically acceptable salt or solvate thereof, wherein R^{3a} is selected from the group consisting of:

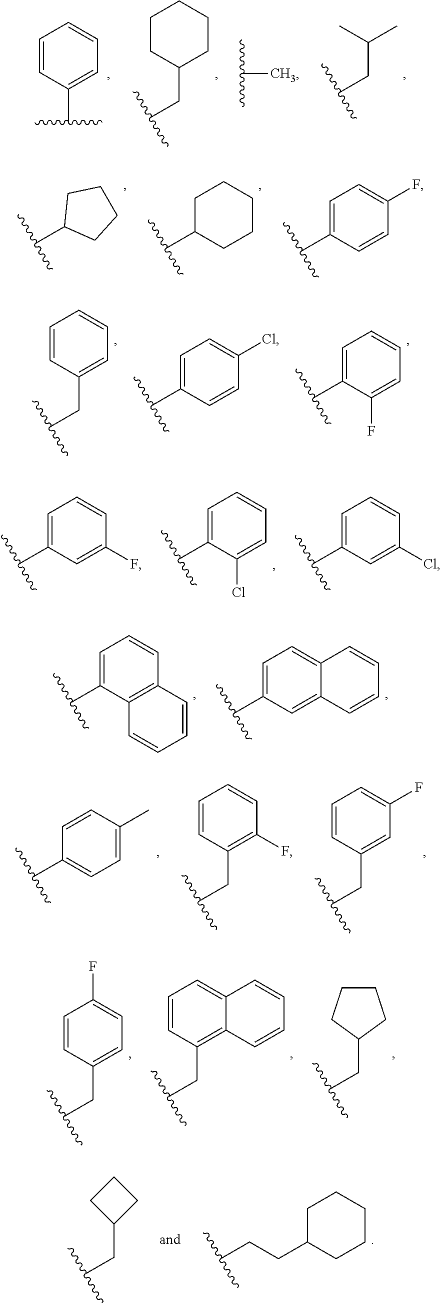

In another embodiment, Compounds of the Disclosure are compounds represented by Formula I, or a pharmaceutically acceptable salt or solvate thereof, wherein R^{3a} is hydrogen and R^{3b} is selected from the group consisting of:

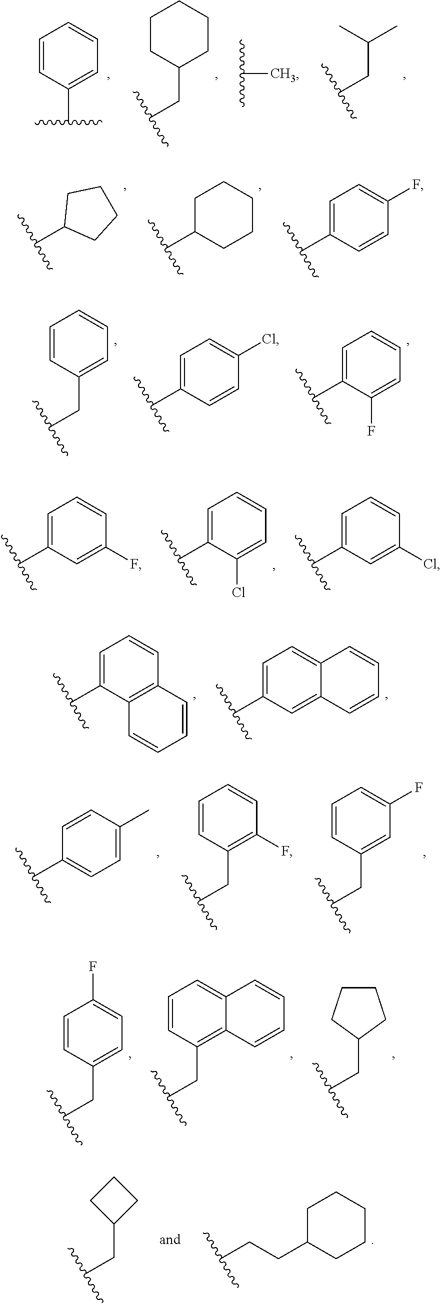

In another embodiment, Compounds of the Disclosure are compounds represented by any one of Formulae I-III, or a pharmaceutically acceptable salt or solvate thereof, wherein R^{3a} is optionally substituted phenyl.

In another embodiment, Compounds of the Disclosure are compounds represented by Formulae I or II, or a pharmaceutically acceptable salt or solvate thereof, wherein R^{4a} is —CH—₂CH₃.

In another embodiment, Compounds of the Disclosure are compounds represented by any one of Formulae I-II, or a pharmaceutically acceptable salt or solvate thereof, wherein R^{5a} is —(CH₂)ₘN(H)C(═NR⁷)NR⁸R⁹. In another embodiment, m is 2 or 3. In another embodiment, R^{5a} is —CH₂CH₂CH₂N(H)C(═NR⁷)NR⁸R⁹. In another embodiment, R⁷ is hydrogen, R⁸ is hydrogen, and R⁹ is selected from the group consisting of hydrogen and C₁₋₄ alkyl. In another embodiment, R^{5a} is —CH₂CH₂CH2N(H)C(═NH)NH₂.

In another embodiment, Compounds of the Disclosure are compounds represented by any one of Formulae I-II, or a pharmaceutically acceptable salt or solvate thereof, wherein R^{5a} is —(CH₂)ₘN(H)R⁹. In another embodiment, m is 2 or 3. In another embodiment, R⁹ is optionally substituted 4- to 8-membered heterocyclo or optionally substituted 5- to 10-membered heteroaryl. In another embodiment, R⁹ is optionally substituted 4- to 8-membered heterocyclo. In another embodiment, R⁹ is optionally substituted 4- to 8-membered heterocyclo selected from the group consisting of:

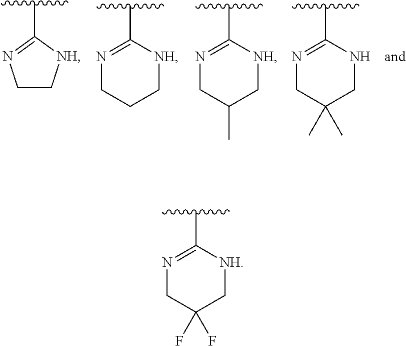

In another embodiment, Compounds of the Disclosure are any one or more of the compounds of Table 1, or a pharmaceutically acceptable salt or solvate thereof. In another embodiment, Compounds of the Disclosure are 2,2,2-trifluoroacetate (TFA) salts. In another embodiment, Compounds of the Disclosure are hydrochloric acid (HCl) salts.

TABLE 1

| Cpd. | | |
|---|---|---|
| No. | Structure | Name |
| 1 | 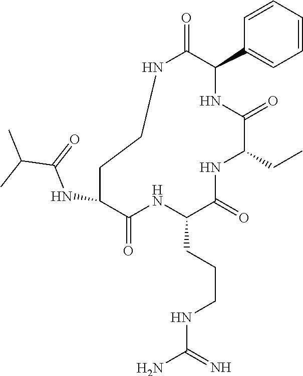 [SMILES: CC[C@@H]1NC(=O)[C@H](CCCNC(=N)N)NC(=O)[C@H](NC(=O)C(C)C)CCNC(=O)[C@@H](c2ccccc2)NC1=O] | N-((3R,6S,9S,12R)-6-ethyl-9-(3- guanidinopropyl)-2,5,8,11-tetraoxo-3- phenyl-1,4,7,10-tetraazacyclotetradecan-12- yl)isobutyramide |
| 4 | 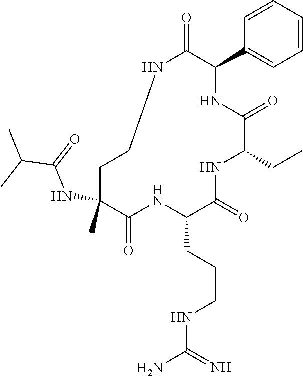 [SMILES: CC[C@@H]1NC(=O)[C@H](CCCNC(=N)N)NC(=O)[C@](C)(NC(=O)C(C)C)CCNC(=O)[C@@H](c2ccccc2)NC1=O] | N-((3R,6S,9S,12R)-6-ethyl-9-(3- guanidinopropyl)-12-methyl-2,5,8,11- tetraoxo-3-phenyl-1,4,7,10- tetraazacyclotetradecan-12-yl)isobutyramide |
| 5 | 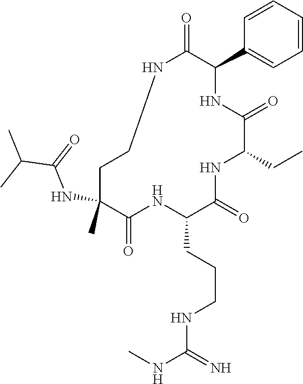 [SMILES: CC[C@@H]1NC(=O)[C@H](CCCNC(=N)NC)NC(=O)[C@](C)(NC(=O)C(C)C)CCNC(=O)[C@@H](c2ccccc2)NC1=O] | N-((3R,6S,9S,12R)-6-ethyl-12-methyl-9-(3- (3-methylguanidino)propyl)-2,5,8,11- tetraoxo-3-phenyl-1,4,7,10- tetraazacyclotetradecan-12-yl)isobutyramide |
| 6 | 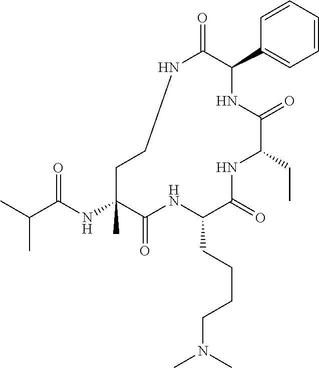 [SMILES: CC[C@@H]1NC(=O)[C@H](CCCCN(C)C)NC(=O)[C@](C)(NC(=O)C(C)C)CCNC(=O)[C@@H](c2ccccc2)NC1=O] | N-((3R,6S,9S,12R)-9-(4- (dimethylamino)butyl)-6-ethyl-12-methyl- 2,5,8,11-tetraoxo-3-phenyl-1,4,7,10- tetraazacyclotetradecan-12-yl)isobutyramide |
| 7 | 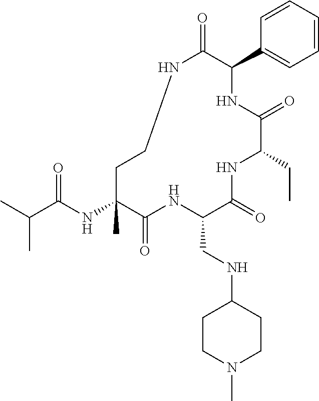 [SMILES: CC[C@@H]1NC(=O)[C@H](CNC2CCN(C)CC2)NC(=O)[C@](C)(NC(=O)C(C)C)CCNC(=O)[C@@H](c2ccccc2)NC1=O] | N-((3R,6S,9S,12R)-6-ethyl-12-methyl-9- (((1-methylpiperidin-4-yl)amino)methyl)- 2,5,8,11-tetraoxo-3-phenyl-1,4,7,10- tetraazacyclotetradecan-12-yl)isobutyramide |
| 8 | 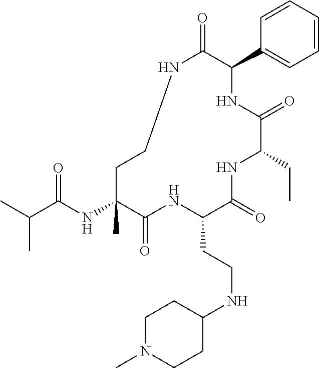 [SMILES: CC[C@@H]1NC(=O)[C@H](CCNC2CCN(C)CC2)NC(=O)[C@](C)(NC(=O)C(C)C)CCNC(=O)[C@@H](c2ccccc2)NC1=O] | N-((3R,6S,9S,12R)-6-ethyl-12-methyl-9-(2- ((1-methylpiperidin-4-yl)amino)ethyl)- 2,5,8,11-tetraoxo-3-phenyl-1,4,7,10- tetraazacyclotetradecan-12-yl)isobutyramide |
| 9 | 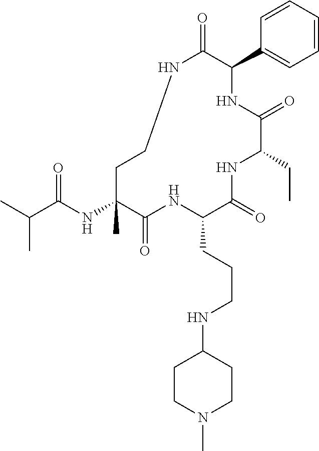 [SMILES: CC[C@@H]1NC(=O)[C@H](CCCNC2CCN(C)CC2)NC(=O)[C@](C)(NC(=O)C(C)C)CCNC(=O)[C@@H](c2ccccc2)NC1=O] | N-((3R,6S,9S,12R)-6-ethyl-12-methyl-9-(3- ((1-methylpiperidin-4-yl)arnino)propyl)- 2,5,8,11-tetraoxo-3-phenyl-1,4,7,10- tetraazacyclotetradecan-12-yl)isobutyramide |
| 10 | 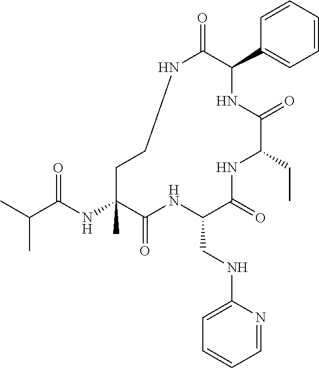 [SMILES: CC[C@@H]1NC(=O)[C@H](CNc2ccccn2)NC(=O)[C@](C)(NC(=O)C(C)C)CCNC(=O)[C@@H](c2ccccc2)NC1=O] | N-((3R,6S,9S,12R)-6-ethyl-12-methyl- 2,5,8,11-tetraoxo-3-phenyl-9-((pyridin-2- ylamino)methyl)-1,4,7,10- tetraazacyclotetradecan-12-yl)isobutyramide |
| 11 | 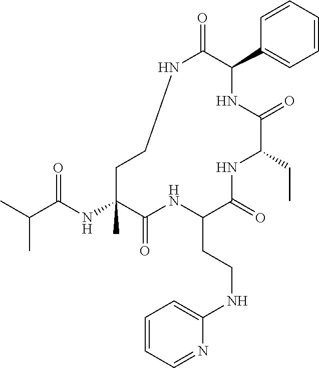 [SMILES: CC[C@@H]1NC(=O)C(CCNc2ccccn2)NC(=O)[C@](C)(NC(=O)C(C)C)CCNC(=O)[C@@H](c2ccccc2)NC1=O] | N-((3R,6S,12R)-6-ethyl-12-methyl-2,5,8,11- tetraoxo-3-phenyl-9-(2-(pyridin-2- ylamino)ethyl)-1,4,7,10- tetraazacyclotetradecan-12-yl)isobutyramide |
| 12 | 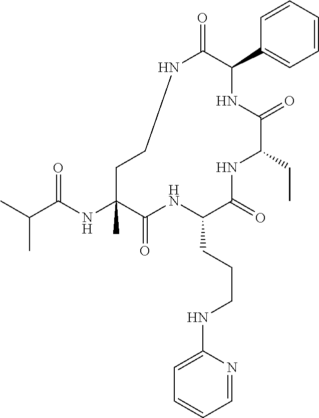 [SMILES: CC[C@@H]1NC(=O)[C@H](CCCNc2ccccn2)NC(=O)[C@](C)(NC(=O)C(C)C)CCNC(=O)[C@@H](c2ccccc2)NC1=O] | N-((3R,6S,9S,12R)-6-ethyl-12-methyl- 2,5,8,11-tetraoxo-3-phenyl-9-(3-(pyridin-2- ylamino)propyl)-1,4,7,10- tetraazacyclotetradecan-12-yl)isobutyramide |
| 13 | 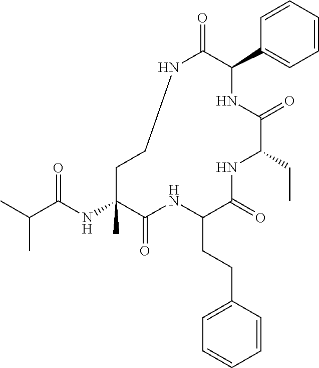 [SMILES: CC[C@@H]1NC(=O)C(CCc2ccccc2)NC(=O)[C@](C)(NC(=O)C(C)C)CCNC(=O)[C@@H](c2ccccc2)NC1=O] | N-((3R,6S,12R)-6-ethyl-12-methyl-2,5,8,11- tetraoxo-9-phenethyl-3-phenyl-1,4,7,10- tetraazacyclotetradecan-12-yl)isobutyramide |
| 14 | 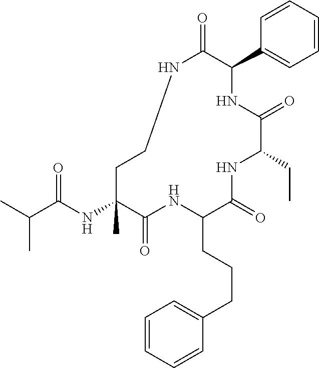 [SMILES: CC[C@@H]1NC(=O)C(CCCc2ccccc2)NC(=O)[C@](C)(NC(=O)C(C)C)CCNC(=O)[C@@H](c2ccccc2)NC1=O] | N-((3R,6S,12R)-6-ethyl-12-methyl-2,5,8,11- tetraoxo-3-phenyl-9-(3-phenylpropyl)- 1,4,7,10-tetraazacyclotetradecan-12- yl)isobutyramide |
| 15 | 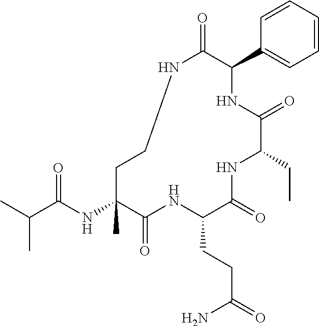 [SMILES: CC[C@@H]1NC(=O)[C@H](CCC(N)=O)NC(=O)[C@](C)(NC(=O)C(C)C)CCNC(=O)[C@@H](c2ccccc2)NC1=O] | N-((3R,6S,12R)-9-(3-amino-3-oxopropyl)-6- ethyl-12-methyl-2,5,8,11-tetraoxo-3-phenyl- 1,4,7,10-tetraazacyclotetradecan-12- yl)isobutyramide |
| 16 | 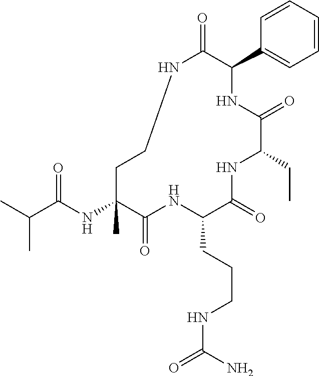 [SMILES: CC[C@@H]1NC(=O)[C@H](CCCNC(N)=O)NC(=O)[C@](C)(NC(=O)C(C)C)CCNC(=O)[C@@H](c2ccccc2)NC1=O] | N-((3R,6S,9S,12R)-6-ethyl-12-methyl- 2,5,8,11-tetraoxo-3-phenyl-9-(3- ureidopropyl)-1,4,7,10- tetraazacyclotetradecan-12-yl)isobutyramide |
| 17 | 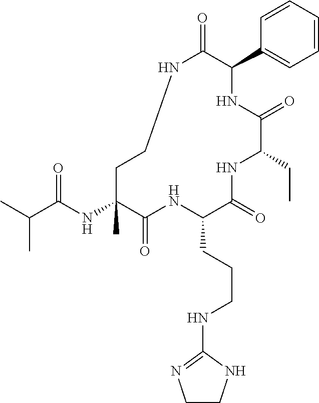 [SMILES: CC[C@@H]1NC(=O)[C@H](CCCNC2=NCCN2)NC(=O)[C@](C)(NC(=O)C(C)C)CCNC(=O)[C@@H](c2ccccc2)NC1=O] | N-((3R,6S,9S,12R)-9-(3-((4,5-dihydro-1H- imidazol-2-yl)amino)propyl)-6-ethyl-12- methyl-2,5,8,11-tetraoxo-3-phenyl-1,4,7,10- tetraazacyclotetradecan-12-yl)isobutyramide |
| 18 | 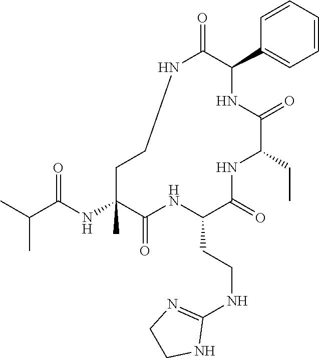 [SMILES: CC[C@@H]1NC(=O)[C@H](CCNC2=NCCN2)NC(=O)[C@](C)(NC(=O)C(C)C)CCNC(=O)[C@@H](c2ccccc2)NC1=O] | N-((3R,6S,9S,12R)-9-(2-((4,5-dihydro-1H- imidazol-2-yl)amino)ethyl)-6-ethyl-12- methyl-2,5,8,11-tetraoxo-3-phenyl-1,4,7,10- tetraazacyclotetradecan-12-yl)isobutyramide |
| 19 | 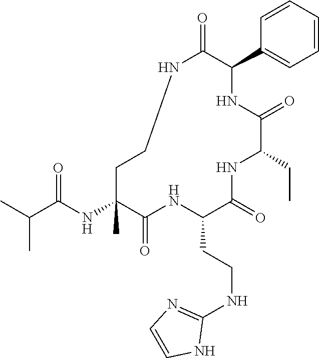 [SMILES: CC[C@@H]1NC(=O)[C@H](CCNc2ncc[nH]2)NC(=O)[C@](C)(NC(=O)C(C)C)CCNC(=O)[C@@H](c2ccccc2)NC1=O] | N-((3R,6S,9S,12R)-9-(2-((1H-imidazol-2- yl)amino)ethyl)-6-ethyl-12-methyl-2,5,8,11- tetraoxo-3-phenyl-1,4,7,10- tetraazacyclotetradecan-12-yl)isobutyramide |
| 20 | 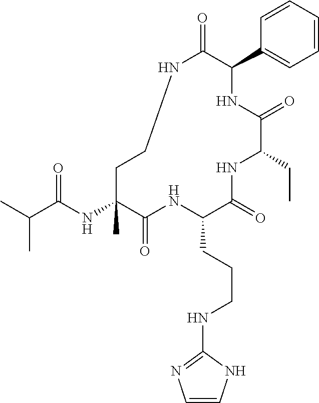 [SMILES: CC[C@@H]1NC(=O)[C@H](CCCNc2ncc[nH]2)NC(=O)[C@](C)(NC(=O)C(C)C)CCNC(=O)[C@@H](c2ccccc2)NC1=O] | N-((3R,6S,9S,12R)-9-(3-((1H-imidazol-2- yl)amino)propyl)-6-ethyl-12-methyl- 2,5,8,11-tetraoxo-3-phenyl-1,4,7,10- tetraazacyclotetradecan-12-yl)isobutyramide |
| 21 | 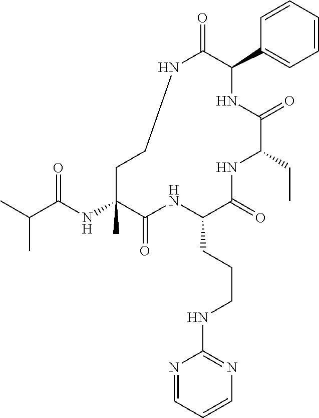 [SMILES: CC[C@@H]1NC(=O)[C@H](CCCNc2ncccn2)NC(=O)[C@](C)(NC(=O)C(C)C)CCNC(=O)[C@@H](c2ccccc2)NC1=O] | N-((3R,6S,12R)-6-ethyl-12-methyl-2,5,8,11- tetraoxo-3-phenyl-9-(3-(pyrimidin-2- ylamino)propyl)-1,4,7,10- tetraazacyclotetradecan-12-yl)isobutyramide |
| 22 | 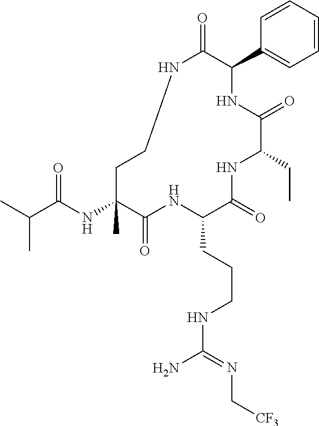 [SMILES: CC[C@@H]1NC(=O)[C@H](CCCN/C(N)=N/CC(F)(F)F)NC(=O)[C@](C)(NC(=O)C(C)C)CCNC(=O)[C@@H](c2ccccc2)NC1=O] | N-((3R,6S,9S,12R)-6-ethyl-12-methyl- 2,5,8,11-tetraoxo-3-phenyl-9-(3-((E)-2- (2,2,2-trifluoroethyl)guanidino)propyl)- 1,4,7,10-tetraazacyclotetradecan-12- yl)isobutyramide |
| 23 | 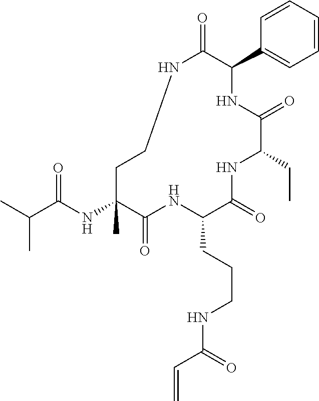 [SMILES: C=CC(=O)NCCC[C@@H]1NC(=O)[C@](C)(NC(=O)C(C)C)CCNC(=O)[C@@H](c2ccccc2)NC(=O)[C@H](CC)NC1=O] | N-(3-((5S,8R,13R)-5-ethyl-13- isobutyramido-13-methyl-3,6,9,14-tetraoxo- 8-phenyl-1,4,7,10-tetraazacyclotetradecan-2- yl)propyl)acrylamide |
| 24 | 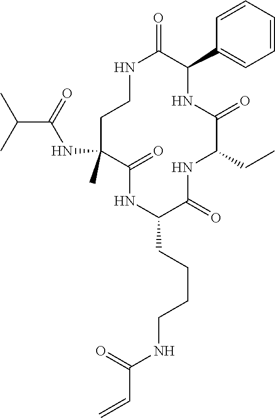 [SMILES: C=CC(=O)NCCCC[C@@H]1NC(=O)[C@](C)(NC(=O)C(C)C)CCNC(=O)[C@@H](c2ccccc2)NC(=O)[C@H](CC)NC1=O] | N-(4-((2S,5S,8R,13R)-5-ethyl-13- isobutyramido-13-methyl-3,6,9,14-tetraoxo- 8-phenyl-1,4,7,10-tetraazacyclotetradecan-2- yl)butyl)acrylamide |
| 25 | 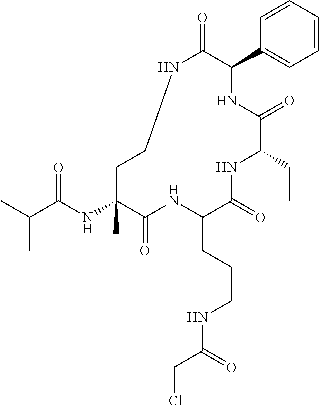 [SMILES: CC[C@@H]1NC(=O)C(CCCNC(=O)CCl)NC(=O)[C@](C)(NC(=O)C(C)C)CCNC(=O)[C@@H](c2ccccc2)NC1=O] | N-((3R,6S,12R)-9-(3-(2- chloroacetamido)propyl)-6-ethyl-12-methyl- 2,5,8,11-tetraoxo-3-phenyl-1,4,7,10- tetraazacyclotetradecan-12-yl)isobutyramide |
| 26 | 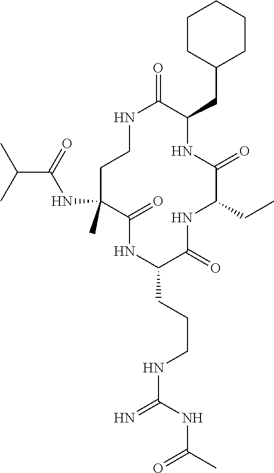 [SMILES: CC[C@@H]1NC(=O)[C@H](CCCNC(=N)NC(C)=O)NC(=O)[C@](C)(NC(=O)C(C)C)CCNC(=O)[C@@H](CC2CCCCC2)NC1=O] | N-((3R,6S,9S,12R)-9-(3-(3- acetylguanidino)propyl)-3- (cyclohexylmethyl)-6-ethyl-12-methyl- 2,5,8,11-tetraoxo-1,4,7,10- tetraazacyclotetradecan-12-yl)isobutyramide |
| 27 | 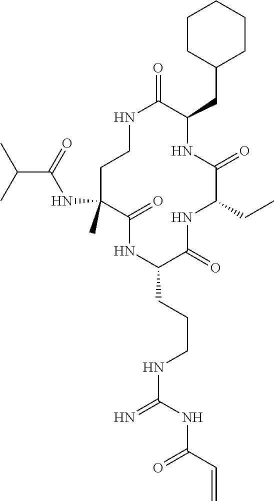 [SMILES: C=CC(=O)NC(=N)NCCC[C@@H]1NC(=O)[C@](C)(NC(=O)C(C)C)CCNC(=O)[C@@H](CC2CCCCC2)NC(=O)[C@H](CC)NC1=O] | N-(N-(3-((2S,5S,8R,13R)-8- (cyclohexylmethyl)-5-ethyl-13- isobutyramido-13-methyl-3,6,9,14-tetraoxo- 1,4,7,10-tetraazacyclotetradecan-2- yl)propyl)carbamimidoyl)acrylamide |
| 28 | 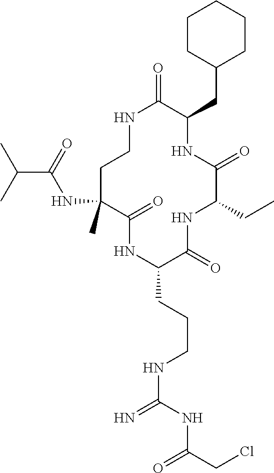 [SMILES: CC[C@@H]1NC(=O)[C@H](CCCNC(=N)NC(=O)CCl)NC(=O)[C@](C)(NC(=O)C(C)C)CCNC(=O)[C@@H](CC2CCCCC2)NC1=O] | N-((3R,6S,9S,12R)-9-(3-(3-(2- chloroacetyl)guanidino)propyl)-3- (cyclohexylmethyl)-6-ethyl-12-methyl- 2,5,8,11-tetraoxo-1,4,7,10- tetraazacyclotetradecan-12-yl)isobutyramide |
| 29 | 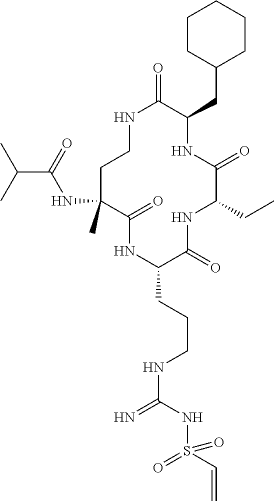 [SMILES: C=CS(=O)(=O)NC(=N)NCCC[C@@H]1NC(=O)[C@](C)(NC(=O)C(C)C)CCNC(=O)[C@@H](CC2CCCCC2)NC(=O)[C@H](CC)NC1=O] | N-((3R,6S,9S,12R)-3-(cyclohexylmethyl)-6- ethyl-12-methyl-2,5,8,11-tetraoxo-9-(3-(3- (vinylsulfonyl)guanidino)propyl)-1,4,7,10- tetraazacyclotetradecan-12-yl)isobutyramide |
| 30 | 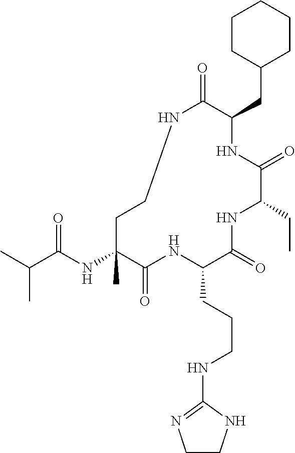 [SMILES: CC[C@@H]1NC(=O)[C@H](CCCNC2=NCCN2)NC(=O)[C@](C)(NC(=O)C(C)C)CCNC(=O)[C@@H](CC2CCCCC2)NC1=O] | N-((3R,6S,9S,12R)-3-(cyclohexylmethyl)-9- (3-((4,5-dihydro-1H-imidazol-2- yl)amino)propyl)-6-ethyl-12-methyl- 2,5,8,11-tetraoxo-1,4,7,10- tetraazacyclotetradecan-12-yl)isobutyramide |
| 31 | 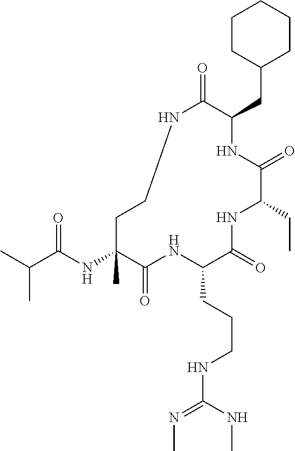 [SMILES: CC[C@@H]1NC(=O)[C@H](CCCN/C(=N/C)NC)NC(=O)[C@](C)(NC(=O)C(C)C)CCNC(=O)[C@@H](CC2CCCCC2)NC1=O] | N-((3R,6S,9S,12R)-3-(cyclohexylmethyl)-9- (3-((E)-2,3-dimethylguanidino)propyl)-6- ethyl-12-methyl-2,5,8,11-tetraoxo-1,4,7,10- tetraazacyclotetradecan-12-yl)isobutyramide |
| 32 | 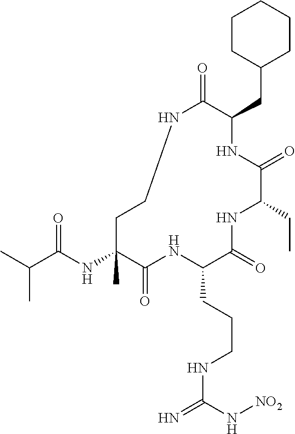 [SMILES: CC[C@@H]1NC(=O)[C@H](CCCNC(=N)N[N+](=O)[O-])NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-9-(3-(3- nitroguanidino)propyl)-2,5,8,11-tetraoxo- 1,4,7,10-tetraazacyclotetradecan-12- yl)isobutyramide |
| 33 | 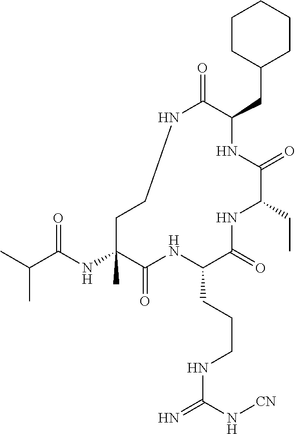 [SMILES: CC[C@@H]1NC(=O)[C@H](CCCNC(=N)NC#N)NC(=O)[C@](C)(NC(=O)C(C)C)CCNC(=O)[C@@H](CC2CCCCC2)NC1=O] | N-((3R,6S,9S,12R)-9-(3-(3- cyanoguanidino)propyl)-3- (cyclohexylmethyl)-6-ethyl-12-methyl- 2,5,8,11-tetraoxo-1,4,7,10- tetraazacyclotetradecan-12-yl)isobutyramide |
| 34 | 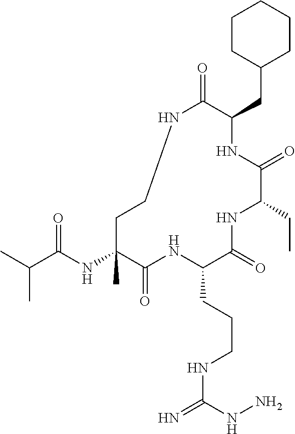 [SMILES: CC[C@@H]1NC(=O)[C@H](CCCNC(=N)NN)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-9-(3- (hydrazinecarboximidamido)propyl)-12- methyl-2,5,8,11-tetraoxo-1,4,7,10- tetraazacyclotetradecan-12-yl)isobutyramide |
| 35 | 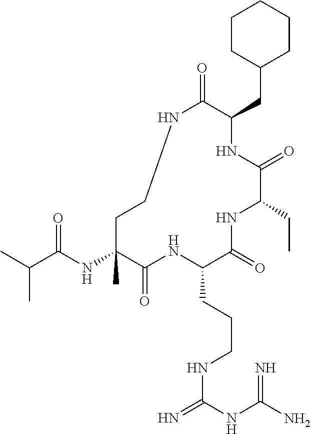 [SMILES: CC[C@@H]1NC(=O)[C@H](CCCNC(=N)NC(=N)N)NC(=O)[C@](C)(NC(=O)C(C)C)CCNC(=O)[C@@H](CC2CCCCC2)NC1=O] | N-((3R,6S,9S,12R)-9-(3-(3- carbamimidoylguanidino)propyl)-3- (cyclohexylmethyl)-6-ethyl-12-methyl- 2,5,8,11-tetraoxo-1,4,7,10- tetraazacyclotetradecan-12-yl)isobutyramide |
| 36 | 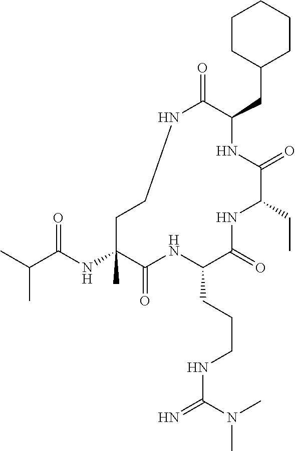 [SMILES: CC[C@@H]1NC(=O)[C@H](CCCNC(=N)N(C)C)NC(=O)[C@](C)(NC(=O)C(C)C)CCNC(=O)[C@@H](CC2CCCCC2)NC1=O] | N-((3R,6S,9S,12R)-3-(cyclohexylmethyl)-9- (3-(3,3-dimethylguanidino)propyl)-6-ethyl- 12-methyl-2,5,8,11-tetraoxo-1,4,7,10- tetraazacyclotetradecan-12-yl)isobutyramide |
| 37 | 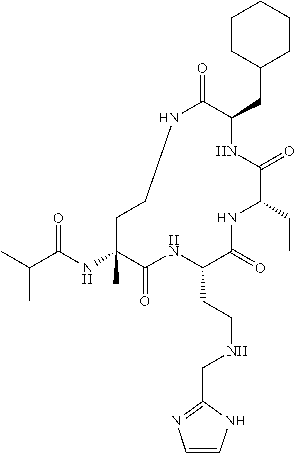 [SMILES: CC[C@@H]1NC(=O)[C@H](CCNCc2ncc[nH]2)NC(=O)[C@](C)(NC(=O)C(C)C)CCNC(=O)[C@@H](CC2CCCCC2)NC1=O] | N-((3R,6S,9S,12R)-9-(2-(((1H-imidazol-2- yl)methyl)amino)ethyl)-3- (cyclohexylmethyl)-6-ethyl-12-methyl- 2,5,8,11-tetraoxo-1,4,7,10- tetraazacyclotetradecan-12-yl)isobutyramide |
| 38 | 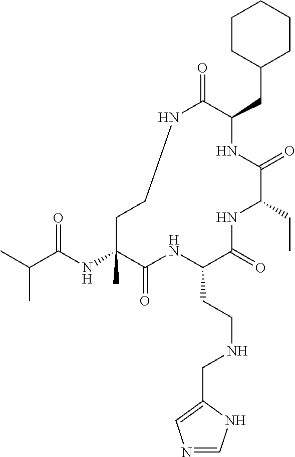 [SMILES: CC[C@@H]1NC(=O)[C@H](CCNCc2cnc[nH]2)NC(=O)[C@](C)(NC(=O)C(C)C)CCNC(=O)[C@@H](CC2CCCCC2)NC1=O] | N-((3R,6S,9S,12R)-9-(2-(((1H-imidazol-5- yl)methyl)amino)ethyl)-3- (cyclohexylmethyl)-6-ethyl-12-methyl- 2,5,8,11-tetraoxo-1,4,7,10- tetraazacyclotetradecan-12-yl)isobutyramide |
| 39 | 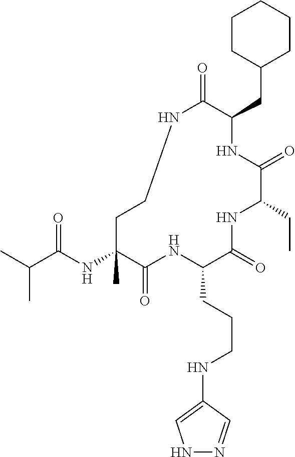 [SMILES: CC[C@@H]1NC(=O)[C@H](CCCNc2cn[nH]c2)NC(=O)[C@](C)(NC(=O)C(C)C)CCNC(=O)[C@@H](CC2CCCCC2)NC1=O] | N-((3R,6S,9S,12R)-9-(3-((1H-pyrazol-4- yl)amino)propyl)-3-(cyclohexylmethyl)-6- ethyl-12-methyl-2,5,8,11-tetraoxo-1,4,7,10- tetraazacyclotetradecan-12-yl)isobutyramide |
| 40 | 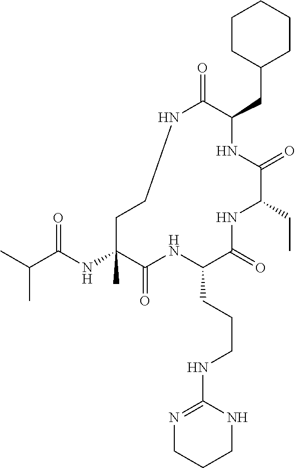 [SMILES: CC[C@@H]1NC(=O)[C@H](CCCNC2=NCCCN2)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-2,5,8,11-tetraoxo-9-(3- ((1,4,5,6-tetrahydropyrimidin-2- yl)amino)propyl)-1,4,7,10- tetraazacyclotetradecan-12-yl)isobutyramide |
| 41 | 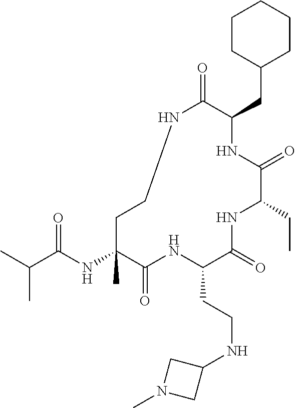 [SMILES: CC[C@@H]1NC(=O)[C@H](CCNC2CN(C)C2)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-9-(2-((1-methylazetidin-3- yl)amino)ethyl)-2,5,8,11-tetraoxo-1,4,7,10- tetraazacyclotetradecan-12-yl)isobutyramide |
| 42 | 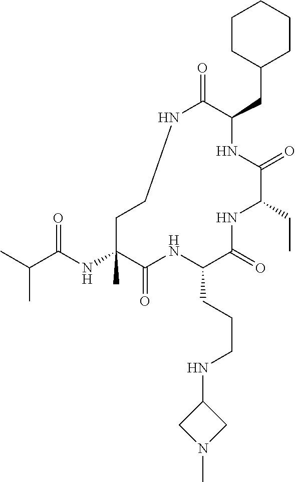 [SMILES: CC[C@@H]1NC(=O)[C@H](CCCNC2CN(C)C2)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-9-(3-((1-methylazetidin-3- yl)amino)propyl)-2,5,8,11-tetraoxo-1,4,7,10- tetraazacyclotetradecan-12-yl)isobutyramide |
| 43 | 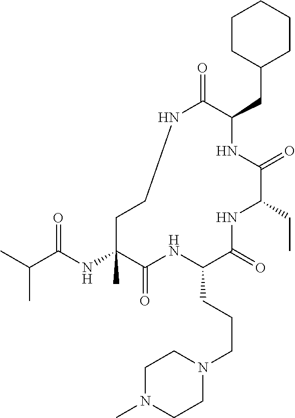 [SMILES: CC[C@@H]1NC(=O)[C@H](CCCN2CCN(C)CC2)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-9-(3-(4-methylpiperazin-1- yl)propyl)-2,5,8,11-tetraoxo-1,4,7,10- tetraazacyclotetradecan-12-yl)isobutyramide |
| 44 | 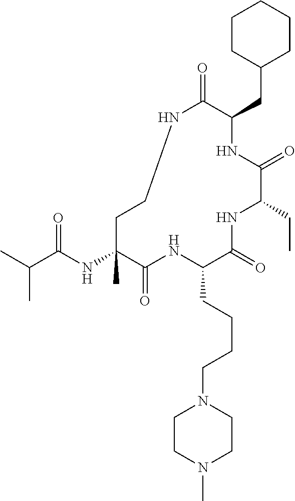 [SMILES: CC[C@@H]1NC(=O)[C@H](CCCCN2CCN(C)CC2)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-9-(4-(4-methylpiperazin-1- yl)butyl)-2,5,8,11-tetraoxo-1,4,7,10- tetraazacyclotetradecan-12-yl)isobutyramide |
| 45 | 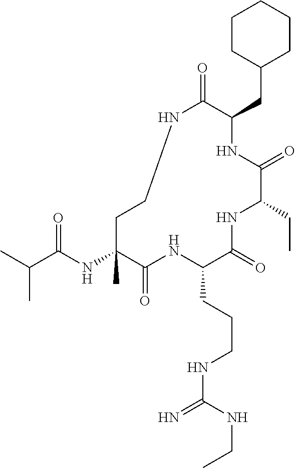 [SMILES: CCNC(=N)NCCC[C@@H]1NC(=O)[C@](C)(NC(=O)C(C)C)CCNC(=O)[C@@H](CC2CCCCC2)NC(=O)[C@H](CC)NC1=O] | N-((3R,6S,9S,12R)-3-(cyclohexylmethyl)-6- ethyl-9-(3-(3-ethylguanidino)propyl)-12- methyl-2,5,8,11-tetraoxo-1,4,7,10- tetraazacyclotetradecan-12-yl)isobutyramide |
| 46 | 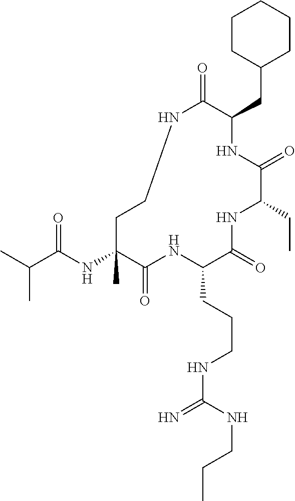 [SMILES: CCCNC(=N)NCCC[C@@H]1NC(=O)[C@](C)(NC(=O)C(C)C)CCNC(=O)[C@@H](CC2CCCCC2)NC(=O)[C@H](CC)NC1=O] | N-((3R,6S,9S,12R)-3-(cyclohexylmethyl)-6- ethyl-12-methyl-2,5,8,11-tetraoxo-9-(3-(3- propylguanidino)propyl)-1,4,7,10- tetraazacyclotetradecan-12-yl)isobutyramide |
| 47 | 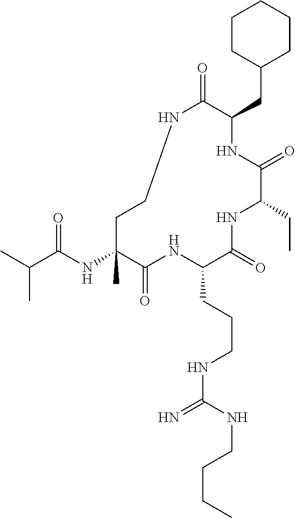 [SMILES: CCCCNC(=N)NCCC[C@@H]1NC(=O)[C@](C)(NC(=O)C(C)C)CCNC(=O)[C@@H](CC2CCCCC2)NC(=O)[C@H](CC)NC1=O] | N-((3R,6S,9S,12R)-9-(3-(3- butylguanidino)propyl)-3- (cyclohexylmethyl)-6-ethyl-12-methyl- 2,5,8,11-tetraoxo-1,4,7,10- tetraazacyclotetradecan-12-yl)isobutyramide |
| 48 | 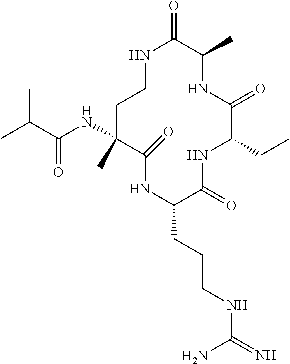 [SMILES: CC[C@@H]1NC(=O)[C@H](CCCNC(=N)N)NC(=O)[C@](C)(NC(=O)C(C)C)CCNC(=O)[C@@H](C)NC1=O] | N-((3R,6S,9S,12R)-6-ethyl-9-(3- guanidinopropyl)-3,12-dimethyl-2,5,8,11- tetraoxo-1,4,7,10-tetraazacyclotetradecan-12- yl)isobutyramide |
| 49 | 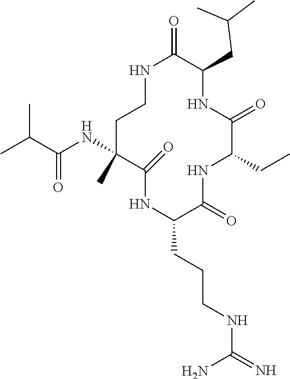 [SMILES: CC[C@@H]1NC(=O)[C@H](CCCNC(=N)N)NC(=O)[C@](C)(NC(=O)C(C)C)CCNC(=O)[C@@H](CC(C)C)NC1=O] | N-((3R,6S,9S,12R)-6-ethyl-9-(3- guanidinopropyl)-3-isobutyl-12-methyl- 2,5,8,11-tetraoxo-1,4,7,10- tetraazacyclotetradecan-12-yl)isobutyramide |
| 50 | 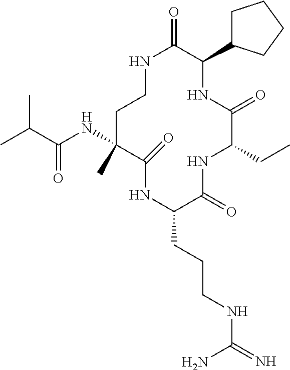 [SMILES: CC[C@@H]1NC(=O)[C@H](CCCNC(=N)N)NC(=O)[C@](C)(NC(=O)C(C)C)CCNC(=O)[C@@H](C2CCCC2)NC1=O] | N-((3R,6S,9S,12R)-3-cyclopentyl-6-ethyl-9- (3-guanidinopropyl)-12-methyl-2,5,8,11- tetraoxo-1,4,7,10-tetraazacyclotetradecan-12- yl)isobutyramide |
| 51 | 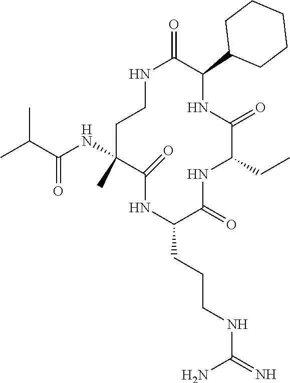 [SMILES: CC[C@@H]1NC(=O)[C@H](CCCNC(=N)N)NC(=O)[C@](C)(NC(=O)C(C)C)CCNC(=O)[C@@H](C2CCCCC2)NC1=O] | N-((3R,6S,9S,12R)-3-cyclohexyl-6-ethyl-9- (3-guanidinopropyl)-12-methyl-2,5,8,11- tetraoxo-1,4,7,10-tetraazacyclotetradecan-12- yl)isobutyramide |
| 52 | 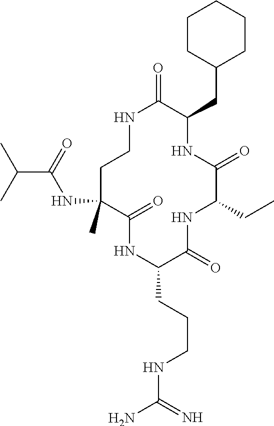 [SMILES: CC[C@@H]1NC(=O)[C@H](CCCNC(=N)N)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-9-(3-guanidinopropyl)-12-methyl- 2,5,8,11-tetraoxo-1,4,7,10- tetraazacyclotetradecan-12-yl)isobutyramide |
| 53 | 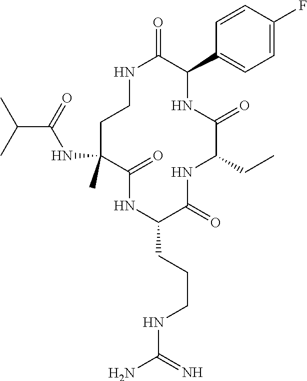 [SMILES: CC[C@@H]1NC(=O)[C@H](CCCNC(=N)N)NC(=O)[C@](C)(NC(=O)C(C)C)CCNC(=O)[C@@H](c2ccc(F)cc2)NC1=O] | N-((3R,6S,9S,12R)-6-ethyl-3-(4- fluorophenyl)-9-(3-guanidinopropyl)-12- methyl-2,5,8,11-tetraoxo-1,4,7,10- tetraazacyclotetradecan-12-yl)isobutyramide |
| 54 | 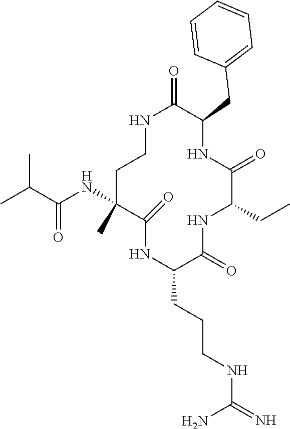 [SMILES: CC[C@@H]1NC(=O)[C@H](CCCNC(=N)N)NC(=O)[C@](C)(NC(=O)C(C)C)CCNC(=O)[C@@H](Cc2ccccc2)NC1=O] | N-((3R,6S,9S,12R)-3-benzyl-6-ethyl-9-(3- guanidinopropyl)-12-methyl-2,5,8,11- tetraoxo-1,4,7,10-tetraazacyclotetradecan-12- yl)isobutyramide |
| 55 | 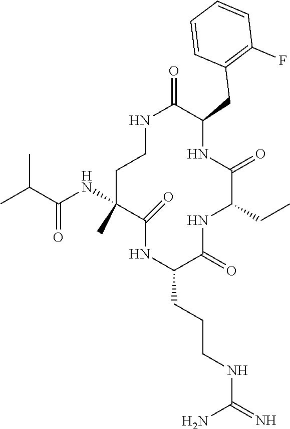 [SMILES: CC[C@@H]1NC(=O)[C@H](CCCNC(=N)N)NC(=O)[C@](C)(NC(=O)C(C)C)CCNC(=O)[C@@H](Cc2ccccc2F)NC1=O] | N-((3R,6S,9S,12R)-6-ethyl-3-(2- fluorobenzyl)-9-(3-guanidinopropyl)-12- methyl-2,5,8,11-tetraoxo-1,4,7,10- tetraazacyclotetradecan-12-yl)isobutyramide |
| 56 | 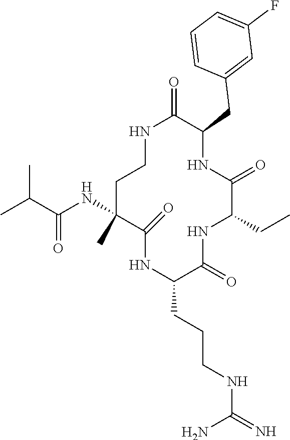 [SMILES: CC[C@@H]1NC(=O)[C@H](CCCNC(=N)N)NC(=O)[C@](C)(NC(=O)C(C)C)CCNC(=O)[C@@H](Cc2cccc(F)c2)NC1=O] | N-((3R,6S,9S,12R)-6-ethyl-3-(3- fluorobenzyl)-9-(3-guanidinopropyl)-12- methyl-2,5,8,11-tetraoxo-1,4,7,10- tetraazacyclotetradecan-12-yl)isobutyramide |
| 57 | 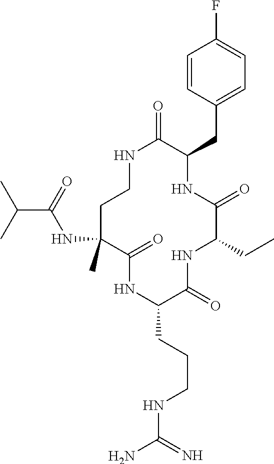 [SMILES: CC[C@@H]1NC(=O)[C@H](CCCNC(=N)N)NC(=O)[C@](C)(NC(=O)C(C)C)CCNC(=O)[C@@H](Cc2ccc(F)cc2)NC1=O] | N-((3R,6S,9S,12R)-6-ethyl-3-(4- fluorobenzyl)-9-(3-guanidinopropyl)-12- methyl-2,5,8,11-tetraoxo-1,4,7,10- tetraazacyclotetradecan-12-yl)isobutyramide |
| 58 | 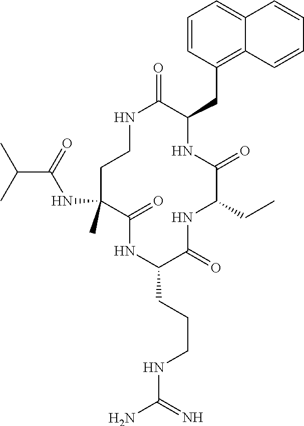 [SMILES: CC[C@@H]1NC(=O)[C@H](CCCNC(=N)N)NC(=O)[C@](C)(NC(=O)C(C)C)CCNC(=O)[C@@H](Cc2cccc3ccccc23)NC1=O] | N-((3R,6S,9S,12R)-6-ethyl-9-(3- guanidinopropyl)-12-methyl-3-(naphthalen- 1-ylmethyl)-2,5,8,11-tetraoxo-1,4,7,10- tetraazacyclotetradecan-12-yl)isobutyramide |
| 59 | 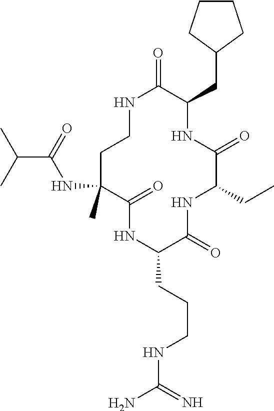 [SMILES: CC[C@@H]1NC(=O)[C@H](CCCNC(=N)N)NC(=O)[C@](C)(NC(=O)C(C)C)CCNC(=O)[C@@H](CC2CCCC2)NC1=O] | N-((3R,6S,9S,12R)-3-(cyclopentylmethyl)-6- ethyl-9-(3-guanidinopropyl)-12-methyl- 2,5,8,11-tetraoxo-1,4,7,10- tetraazacyclotetradecan-12-yl)isobutyramide |
| 60 | 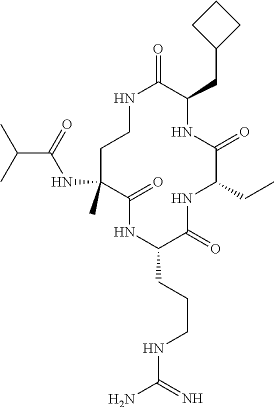 [SMILES: CC[C@@H]1NC(=O)[C@H](CCCNC(=N)N)NC(=O)[C@](C)(NC(=O)C(C)C)CCNC(=O)[C@@H](CC2CCC2)NC1=O] | N-((3R,6S,9S,12R)-3-(cyclobutylmethyl)-6- ethyl-9-(3-guanidinopropyl)-12-methyl- 2,5,8,11-tetraoxo-1,4,7,10- tetraazacyclotetradecan-12-yl)isobutyramide |
| 61 | 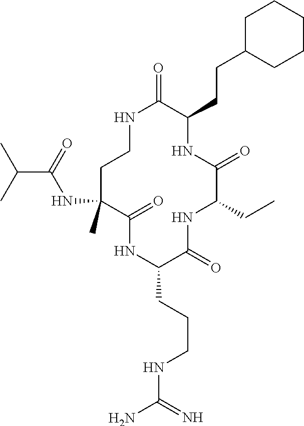 [SMILES: CC[C@@H]1NC(=O)[C@H](CCCNC(=N)N)NC(=O)[C@](C)(NC(=O)C(C)C)CCNC(=O)[C@@H](CCC2CCCCC2)NC1=O] | N-((3R,6S,9S,12R)-3-(2-cyclohexylethyl)-6- ethyl-9-(3-guanidinopropyl)-12-methyl- 2,5,8,11-tetraoxo-1,4,7,10- tetraazacyclotetradecan-12-yl)isobutyramide |
| 62 | 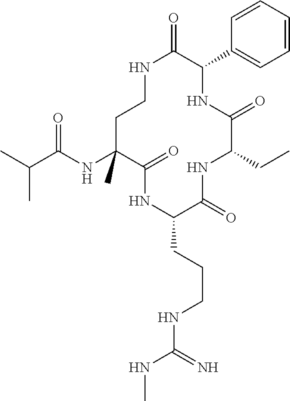 [SMILES: CC[C@@H]1NC(=O)[C@H](CCCNC(=N)NC)NC(=O)[C@](C)(NC(=O)C(C)C)CCNC(=O)[C@H](c2ccccc2)NC1=O] | N-((3S,6S,9S,12R)-6-ethyl-12-methyl-9-(3- (3-methylguanidino)propyl)-2,5,8,11- tetraoxo-3-phenyl-1,4,7,10- tetraazacyclotetradecan-12-yl)isobutyramide |
| 78 | 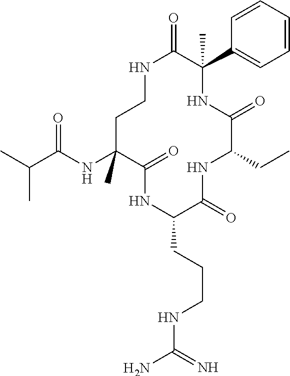 [SMILES: CC[C@@H]1NC(=O)[C@H](CCCNC(=N)N)NC(=O)[C@](C)(NC(=O)C(C)C)CCNC(=O)[C@@](C)(c2ccccc2)NC1=O] | N-((3R,6S,9S,12R)-6-ethyl-9-(3- guanidinopropyl)-3,12-dimethyl-2,5,8,11- tetraoxo-3-phenyl-1,4,7,10- tetraazacyclotetradecan-12-yl)isobutyramide |
| 79 | 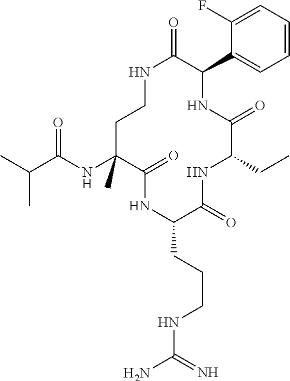 [SMILES: CC[C@@H]1NC(=O)[C@H](CCCNC(=N)N)NC(=O)[C@](C)(NC(=O)C(C)C)CCNC(=O)[C@@H](c2ccccc2F)NC1=O] | N-((3R,6S,9S,12R)-6-ethyl-3-(2- fluorophenyl)-9-(3-guanidinopropyl)-12- methyl-2,5,8,11-tetraoxo-1,4,7,10- tetraazacyclotetradecan-12-yl)isobutyramide |
| 80 | 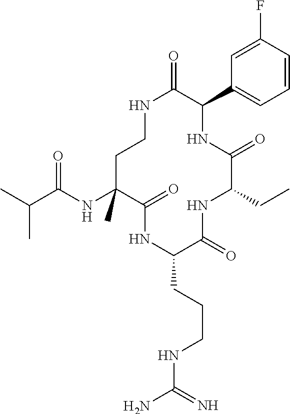 [SMILES: CC[C@@H]1NC(=O)[C@H](CCCNC(=N)N)NC(=O)[C@](C)(NC(=O)C(C)C)CCNC(=O)[C@@H](c2cccc(F)c2)NC1=O] | N-((3R,6S,9S,12R)-6-ethyl-3-(3- fluorophenyl)-9-(3-guanidinopropyl)-12- methyl-2,5,8,11-tetraoxo-1,4,7,10- tetraazacyclotetradecan-12-yl)isobutyramide |
| 81 | 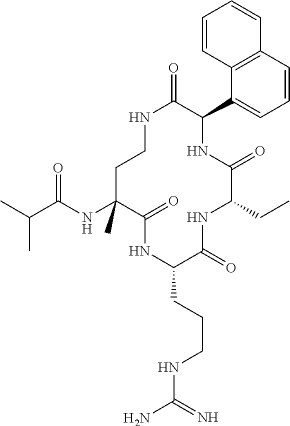 [SMILES: CC[C@@H]1NC(=O)[C@H](CCCNC(=N)N)NC(=O)[C@](C)(NC(=O)C(C)C)CCNC(=O)[C@@H](c2cccc3ccccc23)NC1=O] | N-((3R,6S,9S,12R)-6-ethyl-9-(3- guanidinopropyl)-12-methyl-3-(naphthalen- 1-yl)-2,5,8,11-tetraoxo-1,4,7,10- tetraazacyclotetradecan-12-yl)isobutyramide |
| 82 | 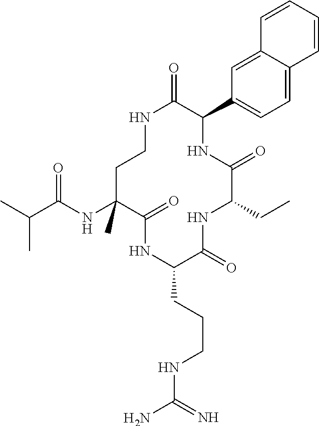 [SMILES: CC[C@@H]1NC(=O)[C@H](CCCNC(=N)N)NC(=O)[C@](C)(NC(=O)C(C)C)CCNC(=O)[C@@H](c2ccc3ccccc3c2)NC1=O] | N-((3R,6S,9S,12R)-6-ethyl-9-(3- guanidinopropyl)-12-methyl-3-(naphthalen- 2-yl)-2,5,8,11-tetraoxo-1,4,7,10- tetraazacyclotetradecan-12-yl)isobutyramide |
| 83 | 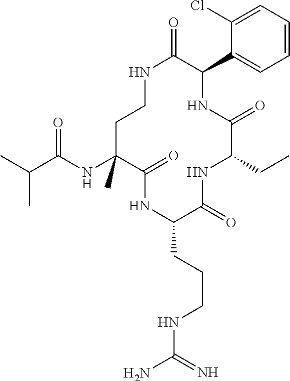 [SMILES: CC[C@@H]1NC(=O)[C@H](CCCNC(=N)N)NC(=O)[C@](C)(NC(=O)C(C)C)CCNC(=O)[C@@H](c2ccccc2Cl)NC1=O] | N-((3R,6S,9S,12R)-3-(2-chlorophenyl)-6- ethyl-9-(3-guanidinopropyl)-12-methyl- 2,5,8,11-tetraoxo-1,4,7,10- tetraazacyclotetradecan-12-yl)isobutyramide |
| 84 | 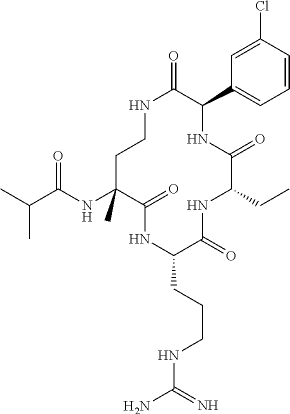 [SMILES: CC[C@@H]1NC(=O)[C@H](CCCNC(=N)N)NC(=O)[C@](C)(NC(=O)C(C)C)CCNC(=O)[C@@H](c2cccc(Cl)c2)NC1=O] | N-((3R,6S,9S,12R)-3-(3-chlorophenyl)-6- ethyl-9-(3-guanidinopropyl)-12-methyl- 2,5,8,11-tetraoxo-1,4,7,10- tetraazacyclotetradecan-12-yl)isobutyramide |
| 85 | 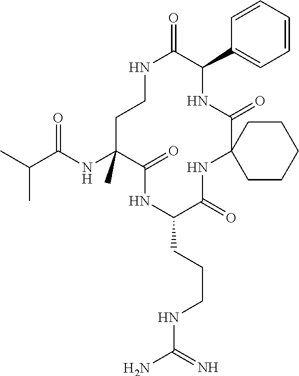 [SMILES: CC(C)C(=O)N[C@]1(C)CCNC(=O)[C@@H](c2ccccc2)NC(=O)C2(CCCCC2)NC(=O)[C@H](CCCNC(=N)N)NC1=O] | N-((9S,12R,17R)-9-(3-guanidinopropyl)-12- methyl-8,11,16,19-tetraoxo-17-phenyl- 7,10,15,18-tetraazaspiro[5.13]nonadecan-12- yl)isobutyramide |
| 86 | 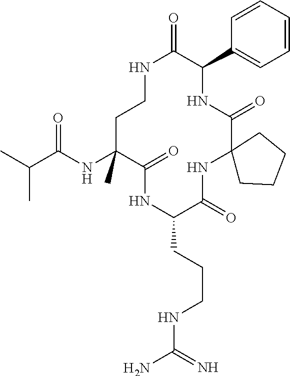 [SMILES: CC(C)C(=O)N[C@]1(C)CCNC(=O)[C@@H](c2ccccc2)NC(=O)C2(CCCC2)NC(=O)[C@H](CCCNC(=N)N)NC1=O] | N-((8S,11R,16R)-8-(3-guanidinopropyl)-11- methyl-7,10,15,18-tetraoxo-16-phenyl- 6,9,14,17-tetraazaspiro[4.13]octadecan-11- yl)isobutyramide |
| 87 | 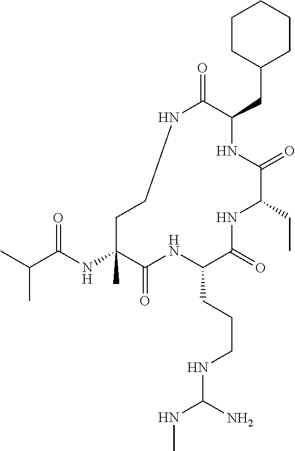 [SMILES: CC[C@@H]1NC(=O)[C@H](CCCNC(N)NC)NC(=O)[C@](C)(NC(=O)C(C)C)CCNC(=O)[C@@H](CC2CCCCC2)NC1=O] | N-((3R,6S,9S,12R)-9-(3- ((amino(methylamino)methyl)amino)propyl)- 3-(cyclohexylmethyl)-6-ethyl-12-methyl- 2,5,8,11-tetraoxo-1,4,7,10- tetraazacyclotetradecan-12-yl)isobutyramide |
| 88 | 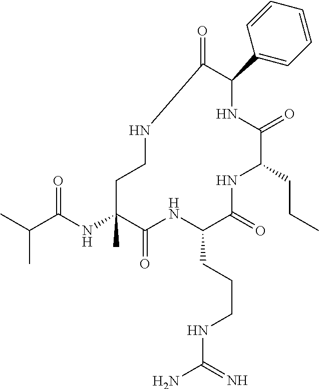 [SMILES: CCC[C@@H]1NC(=O)[C@H](CCCNC(=N)N)NC(=O)[C@](C)(NC(=O)C(C)C)CCNC(=O)[C@@H](c2ccccc2)NC1=O] | N-((3R,6S,9S,12R)-9-(3-guanidinopropyl)- 12-methyl-2,5,8,11-tetraoxo-3-phenyl-6- propyl-1,4,7,10-tetraazacyclotetradecan-12- yl)isobutyramide |
| 89 | 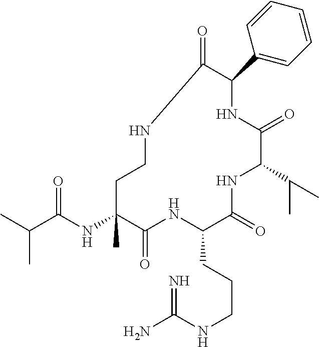 [SMILES: CC(C)C(=O)N[C@]1(C)CCNC(=O)[C@@H](c2ccccc2)NC(=O)[C@H](C(C)C)NC(=O)[C@H](CCCNC(=N)N)NC1=O] | N-((3R,6S,9S,12R)-9-(3-guanidinopropyl)-6- isopropyl-12-methyl-2,5,8,11-tetraoxo-3- phenyl-1,4,7,10-tetraazacyclotetradecan-12- yl)isobutyramide |
| 90 | 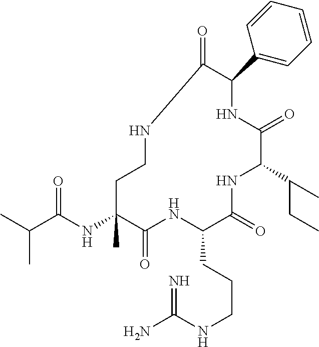 [SMILES: CCC(C)[C@@H]1NC(=O)[C@H](CCCNC(=N)N)NC(=O)[C@](C)(NC(=O)C(C)C)CCNC(=O)[C@@H](c2ccccc2)NC1=O] | N-((3R,6S,9S,12R)-6-((S)-sec-butyl)-9-(3- guanidinopropyl)-12-methyl-2,5,8,11- tetraoxo-3-phenyl-1,4,7,10- tetraazacyclotetradecan-12-yl)isobutyramide |
| 91 | 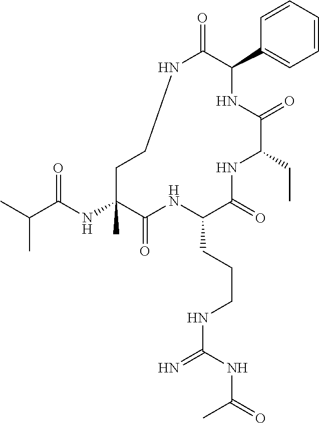 [SMILES: CC[C@@H]1NC(=O)[C@H](CCCNC(=N)NC(C)=O)NC(=O)[C@](C)(NC(=O)C(C)C)CCNC(=O)[C@@H](c2ccccc2)NC1=O] | N-((3R,6S,9S,12R)-9-(3-(3- acetylguanidino)propyl)-6-ethyl-12-methyl- 2,5,8,11-tetraoxo-3-phenyl-1,4,7,10- tetraazacyclotetradecan-12-yl)isobutyramide |
| 92 | 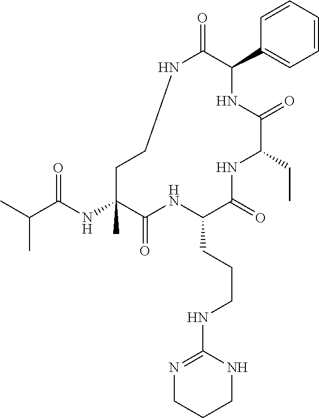 [SMILES: CC[C@@H]1NC(=O)[C@H](CCCNC2=NCCCN2)NC(=O)[C@](C)(NC(=O)C(C)C)CCNC(=O)[C@@H](c2ccccc2)NC1=O] | N-((3R,6S,9S,12R)-6-ethyl-12-methyl- 2,5,8,11-tetraoxo-3-phenyl-9-(3-((1,4,5,6- tetrahydropyrimidin-2-yl)amino)propyl)- 1,4,7,10-tetraazacyclotetradecan-12- yl)isobutyramide |
| 93 | 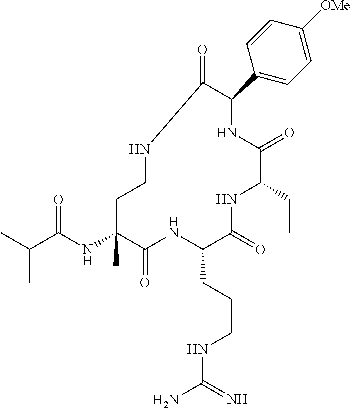 [SMILES: CC[C@@H]1NC(=O)[C@H](CCCNC(=N)N)NC(=O)[C@](C)(NC(=O)C(C)C)CCNC(=O)[C@@H](c2ccc(OC)cc2)NC1=O] | N-((3R,6S,9S,12R)-6-ethyl-9-(3- guanidinopropyl)-3-(4-methoxyphenyl)-12- methyl-2,5,8,11-tetraoxo-1,4,7,10- tetraazacyclotetradecan-12-yl)isobutyramide |
| 94 | 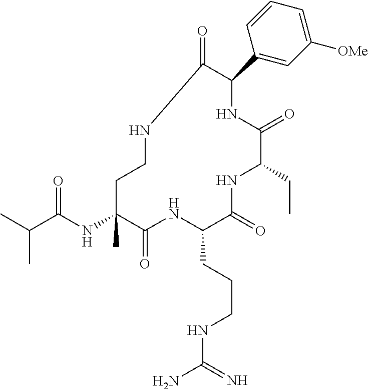 [SMILES: CC[C@@H]1NC(=O)[C@H](CCCNC(=N)N)NC(=O)[C@](C)(NC(=O)C(C)C)CCNC(=O)[C@@H](c2cccc(OC)c2)NC1=O] | N-((3R,6S,9S,12R)-6-ethyl-9-(3- guanidinopropyl)-3-(3-methoxyphenyl)-12- methyl-2,5,8,11-tetraoxo-1,4,7,10- tetraazacyclotetradecan-12-yl)isobutyramide |
| 95 | 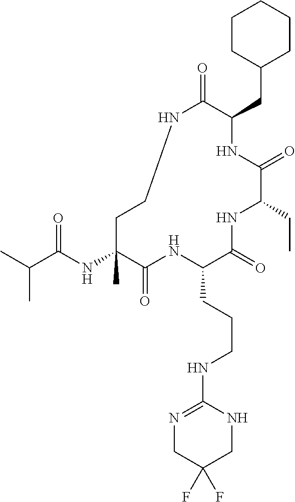 [SMILES: CC[C@@H]1NC(=O)[C@H](CCCNC2=NCC(F)(F)CN2)NC(=O)[C@](C)(NC(=O)C(C)C)CCNC(=O)[C@@H](CC2CCCCC2)NC1=O] | N-((3R,6S,9S,12R)-3-(cyclohexylmethyl)-9- (3-((5,5-difluoro-1,4,5,6- tetrahydropyrimidin-2-yl)amino)propyl)-6- ethyl-12-methyl-2,5,8,11-tetraoxo-1,4,7,10- tetraazacyclotetradecan-12-yl)isobutyramide |
| 96 | 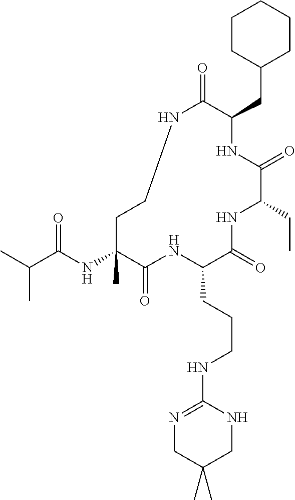 [SMILES: CC[C@@H]1NC(=O)[C@H](CCCNC2=NCC(C)(C)CN2)NC(=O)[C@](C)(NC(=O)C(C)C)CCNC(=O)[C@@H](CC2CCCCC2)NC1=O] | N-((3R,6S,9S,12R)-3-(cyclohexylmethyl)-9- (3-((5,5-dimethyl-1,4,5,6- tetrahydxopyrimidin-2-yl)amino)propyl)-6- ethyl-12-methyl-2,5,8,11-tetraoxo-1,4,7,10- tetraazacyclotetradecan-12-yl)isobutyramide |
| 97 | 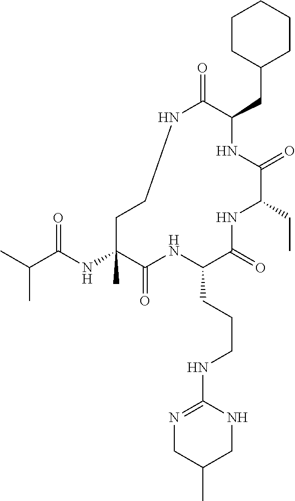 [SMILES: CC[C@@H]1NC(=O)[C@H](CCCNC2=NCC(C)CN2)NC(=O)[C@](C)(NC(=O)C(C)C)CCNC(=O)[C@@H](CC2CCCCC2)NC1=O] | N-((3R,6S,9S,12R)-3-(cyclohexylmethyl)-6- ethyl-12-methyl-9-(3-((5-methyl-1,4,5,6- tetrahydropyrimidin-2-yl)amino)propyl)- 2,5,8,11-tetraoxo-1,4,7,10- tetraazacyclotetradecan-12-yl)isobutyramide |
| 98 | 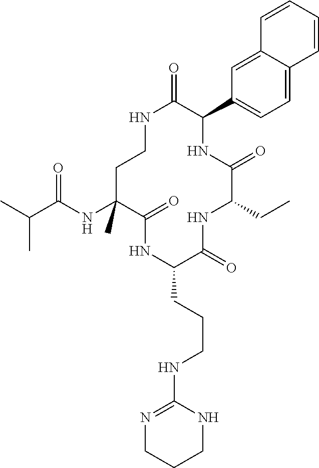 [SMILES: CC[C@@H]1NC(=O)[C@H](CCCNC2=NCCCN2)NC(=O)[C@](C)(NC(=O)C(C)C)CCNC(=O)[C@@H](c2ccc3ccccc3c2)NC1=O] | N-((3R,6S,9S,12R)-6-ethyl-12-methyl-3- (naphthalen-2-yl)-2,5,8,11-tetraoxo-9-(3- ((1,4,5,6-tetrahydropyrimidin-2- yl)amino)propyl)-1,4,7,10- tetraazacyclotetradecan-12-yl)isobutyramide |
| 99 | 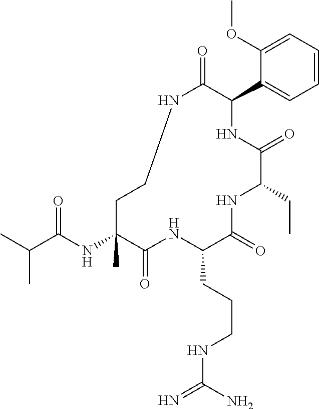 [SMILES: CC[C@@H]1NC(=O)[C@H](CCCNC(=N)N)NC(=O)[C@](C)(NC(=O)C(C)C)CCNC(=O)[C@@H](c2ccccc2OC)NC1=O] | N-((3R,6S,9S,12R)-6-ethyl-9-(3- guanidinopropyl)-3-(2-methoxyphenyl)-12- methyl-2,5,8,11-tetraoxo-1,4,7,10- tetraazacyclotetradecan-12-yl)isobutyramide |
| 100 | 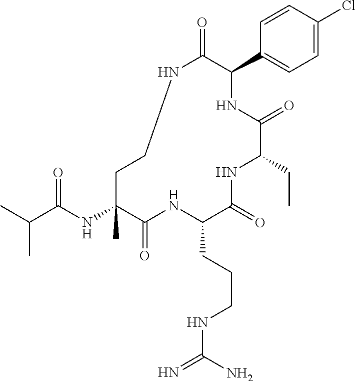 [SMILES: CC[C@@H]1NC(=O)[C@H](CCCNC(=N)N)NC(=O)[C@](C)(NC(=O)C(C)C)CCNC(=O)[C@@H](c2ccc(Cl)cc2)NC1=O] | N-((3R,6S,9S,12R)-3-(4-chlorophenyl)-6- ethyl-9-(3-guanidinopropyl)-12-methyl- 2,5,8,11-tetraoxo-1,4,7,10- tetraazacyclotetradecan-12-yl)isobutyramide |
| 101 | 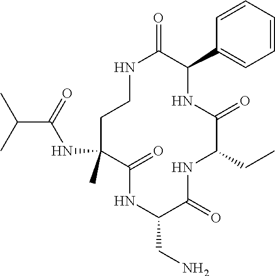 [SMILES: CC[C@@H]1NC(=O)[C@H](CN)NC(=O)[C@](C)(NC(=O)C(C)C)CCNC(=O)[C@@H](c2ccccc2)NC1=O] | N-((3R,6S,9S,12R)-9-(aminomethyl)-6- ethyl-12-methyl-2,5,8,11-tetraoxo-3-phenyl- 1,4,7,10-tetraazacyclotetradecan-12- yl)isobutyramide |
| 102 | 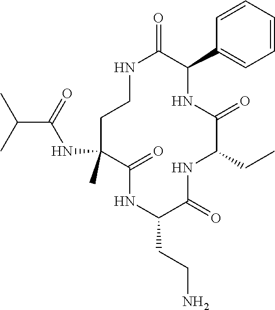 [SMILES: CC[C@@H]1NC(=O)[C@H](CCN)NC(=O)[C@](C)(NC(=O)C(C)C)CCNC(=O)[C@@H](c2ccccc2)NC1=O] | N-((3R,6S,9S,12R)-9-(2-aminoethyl)-6- ethyl-12-methyl-2,5,8,11-tetraoxo-3-phenyl- 1,4,7,10-tetraazacyclotetradecan-12- yl)isobutyramide |
| 103 | 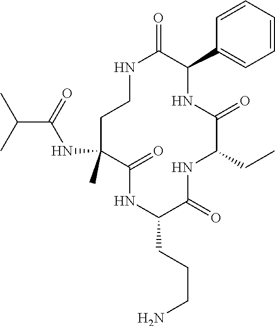 [SMILES: CC[C@@H]1NC(=O)[C@H](CCCN)NC(=O)[C@](C)(NC(=O)C(C)C)CCNC(=O)[C@@H](c2ccccc2)NC1=O] | N-((3R,6S,9S,12R)-9-(3-aminopropyl)-6- ethyl-12-methyl-2,5,8,11-tetraoxo-3-phenyl- 1,4,7,10-tetraazacyclotetradecan-12- yl)isobutyramide |
| 104 | 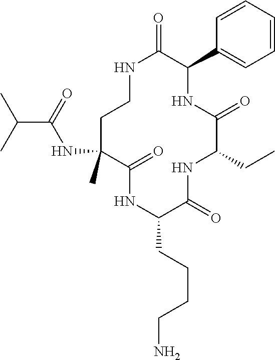 [SMILES: CC[C@@H]1NC(=O)[C@H](CCCCN)NC(=O)[C@](C)(NC(=O)C(C)C)CCNC(=O)[C@@H](c2ccccc2)NC1=O] | N-((3R,6S,9S,12R)-9-(4-aminobutyl)-6- ethyl-12-methyl-2,5,8,11-tetraoxo-3-phenyl- 1,4,7,10-tetraazacyclotetradecan-12- yl)isobutyramide |
| 105 | 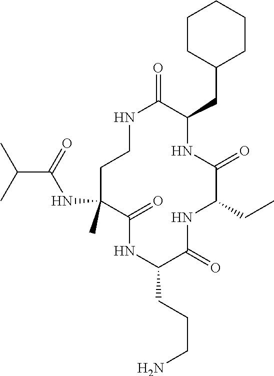 [SMILES: CC[C@@H]1NC(=O)[C@H](CCCN)NC(=O)[C@](C)(NC(=O)C(C)C)CCNC(=O)[C@@H](CC2CCCCC2)NC1=O] | N-((3R,6S,9S,12R)-9-(3-aminopropyl)-3- (cyclohexylmethyl)-6-ethyl-12-methyl- 2,5,8,11-tetraoxo-1,4,7,10- tetraazacyclotetradecan-12-yl)isobutyramide |

Compounds of the Disclosure inhibit the WDR5-binding partner protein-protein interaction e.g., the WDR5-MLL protein-protein interaction, and are thus useful in the treatment or prevention of a variety of diseases and conditions. In particular, Compounds of the Disclosure are useful in methods of treating or preventing a disease or condition wherein inhibition of the WDR5-MLL protein-protein interaction provides a benefit, for example, cancers and proliferative diseases. The therapeutic methods of this disclosure comprise administering a therapeutically effective amount of a Compound of the Disclosure to a subject in need thereof. The present methods also encompass administering a second therapeutic agent to the subject in addition to the Compound of the Disclosure. The second therapeutic agent is selected from drugs known as useful in treating the disease or condition afflicting the subject in need thereof, e.g., a chemotherapeutic agent and/or radiation known as useful in treating a particular cancer.

Certain of the Compounds of the Disclosure may exist as stereoisomers, i.e., isomers that differ only in the spatial arrangement of atoms, including optical isomers and conformational isomers (or conformers) and tautomers. The disclosure includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” or “racemate” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs}, is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry. Certain compounds of the Disclosure can have an ee of about 70% or more, e.g., about 80% or more, about 90% or more, about 91% or more, about 92% or more, about 93% or more, about 94% or more, about 95% or more, about 96% or more, about 97% or more, about 98% or more, or about 99% or more.

The terms “enantiomerically pure” or “enantiopure” refer to a sample of a chiral substance all of whose molecules (within the limits of detection) have the same chirality sense.

The terms “enantiomerically enriched” or “enantioenriched” refer to a sample of a chiral substance whose enantiomeric ratio is greater than 50:50. Enantiomerically enriched compounds may be enantiomerically pure. Certain compounds of the Disclosure are enantioenriched.

Salts and solvates, e.g., hydrates, of the Compounds of the Disclosure can also be used in the methods disclosed herein.

The present disclosure encompasses the preparation and use of salts of Compounds of the Disclosure. As used herein, a “pharmaceutically acceptable salt” refers to salts or zwitterionic forms of Compounds of the Disclosure. Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure.

One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The present disclosure provides Compounds of the Disclosure as inhibitors of the WDR5-binding partner protein-protein interaction, e.g., the WDR5-MLL protein-protein interaction, for the treatment of a variety of diseases and conditions wherein inhibition has a beneficial effect, e.g., cancer. Compounds of the Disclosure typically have a binding affinity (IC₅₀) to WDR5 of less than 100 μM, e.g., less than about 50 μM, less than about 25 μM, and less than about 5 μM, less than about 1 μM, less than about 0.5 μM, less than about 0.1 μM, less than about 0.05 μM, less than about 0.01 μM, less than about 0.005 μM, or less than about 0.001 μM. In one embodiment, the present disclosure relates to a method of treating a subject suffering from a disease or condition wherein inhibition of the WDR5-MLL protein-protein interaction provides a benefit comprising administering a therapeutically effective amount of a Compound of the Disclosure to a subject in need thereof.

Since Compounds of the Disclosure are inhibitors of the WDR5-binding partner protein-protein interaction, e.g., the WDR5-MLL protein-protein interaction, a number of diseases and conditions can be treated by employing these compounds. The present disclosure is thus directed generally to a method for treating a condition or disorder responsive to inhibition of the WDR5-MLL protein-protein interaction, in a subject, e.g., an animal, e.g., a human patient, suffering from, or at risk of suffering from, the condition or disorder, the method comprising administering to the subject an effective amount of one or more Compounds of the Disclosure. In one embodiment, the subject to be treated by the Compound of the Disclosure is a human cancer patient.

The present disclosure is also directed to a method of inhibiting the WDR5-binding partner protein-protein interaction, e.g., the WDR5-MLL protein-protein interaction, in an animal, e.g., a human cancer patient, in need thereof, said method comprising administering to the animal an effective amount of at least one Compound of the Disclosure.

The methods of the present disclosure can be accomplished by administering a Compound of the Disclosure as the neat compound or as a pharmaceutical composition. Administration of a pharmaceutical composition, or neat compound of a Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered.

Further provided are kits comprising a Compound of the Disclosure and, optionally, a second therapeutic agent useful in the treatment of diseases and conditions wherein inhibition of the WDR5-binding partner protein-protein interaction provides a benefit, packaged separately or together, and an insert having instructions for using these active agents.

In one embodiment, a Compound of the Disclosure is administered in conjunction with a second therapeutic agent useful in the treatment of a disease or condition wherein inhibition of the WDR5-binding partner protein-protein interaction provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to a subject in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more dose of the second therapeutic agent can be administered. The Compound of the Disclosure therefore can be used in conjunction with one or more second therapeutic agents, for example, but not limited to, anticancer agents.

Diseases and conditions treatable by the methods of the present disclosure include, but are not limited to, cancer and other proliferative disorders, inflammatory diseases, sepsis, autoimmune disease, and viral infection. In one embodiment, diseases and conditions treatable by the methods of the present disclosure are cancer, a chronic autoimmune disorder, an inflammatory condition, or a proliferative disorder. In one embodiment, a human patient is treated with a Compound of the Disclosure, or a pharmaceutical composition comprising a Compound of the Disclosure, wherein the compound is administered in an amount sufficient to inhibit the WDR5-binding partner protein-protein interaction in the patient.

In one embodiment, the disease to be treated or prevented by the Compound of the Disclosure is cancer. In another embodiment, the present disclosure provides a method of treating or preventing cancer in a subject in need thereof comprising administering a therapeutically effective amount of a Compound of the Disclosure to the subject. While not being limited to a specific mechanism, in some embodiments, Compounds of the Disclosure can treat or prevent cancer by inhibiting the WDR5-MLL protein-protein interaction. Examples of treatable cancers include, but are not limited to, any one or more of the cancers of Table 2.

TABLE 2

| | |
|---|---|
| adrenal cancer | lymphoepithelioma |
| acinic cell carcinoma | lymphoma |
| acoustic neuroma | acute lymphocytic leukemia |
| acral lentigious melanoma | acute myelogeous leukemia |
| acrospiroma | chronic lymphocytic leukemia |
| acute eosinophilic leukemia | liver cancer |
| acute erythroid leukemia | small cell lung cancer |
| acute lymphoblastic leukemia | non-small cell lung cancer |
| acute megakaryoblastic leukemia | MALT lymphoma |
| acute monocytic leukemia | malignant fibrous histiocytoma |
| acute promyelocytic leukemia | malignant peripheral nerve |
| | sheath tumor |
| adenocarcinoma | malignant triton tumor |
| adenoid cystic carcinoma | mantle cell lymphoma |
| adenoma | marginal zone B-cell lymphoma |
| adenomatoid odontogenic tumor | mast cell leukemia |
| adenosquamous carcinoma | mediastinal germ cell tumor |
| adipose tissue neoplasm | medullary carcinoma of the breast |
| adrenocortical carcinoma | medullary thyroid cancer, |
| adult T-cell leukemia/lymphoma | medulloblastoma |
| aggressive NK-cell leukemia | melanoma, |
| AIDS-related lymphoma | meningioma, |
| alveolar rhabdomyosarcoma | merkel cell cancer |
| alveolar soft part sarcoma | mesothelioma |
| ameloblastic fibroma | metastatic urothelial carcinoma |
| anaplastic large cell lymphoma | mixed Mullerian tumor |
| anaplastic thyroid cancer | mucinous tumor |
| angioimmunoblastic T-cell | multiple myeloma |
| lymphoma, | |
| angiomyolipoma | muscle tissue neoplasm |
| angiosarcoma | mycosis fungoides |
| astrocytoma | myxoid liposarcoma |
| atypical teratoid rhabdoid tumor | myxoma |
| B-cell chronic lymphocytic | myxosarcoma |
| leukemia | |
| B-cell prolymphocytic leukemia | nasopharyngeal carcinoma |
| B-cell lymphoma | neurinoma |
| basal cell carcinoma | neuroblastoma |
| biliary tract cancer | neurofibroma |
| bladder cancer | neuroma |
| blastoma | nodular melanoma |
| bone cancer | ocular cancer |
| Brenner tumor | oligoastrocytoma |
| Brown tumor | oligodendroglioma |
| Burkitt's lymphoma | oncocytoma |
| breast cancer | optic nerve sheath meningioma |
| brain cancer | optic nerve tumor |
| carcinoma | oral cancer |
| carcinoma in situ | osteosarcoma |
| carcinosarcoma | ovarian cancer |
| cartilage tumor | Pancoast tumor |
| cementoma | papillary thyroid cancer |
| myeloid sarcoma | paraganglioma |
| chondroma | pinealoblastoma |
| chordoma | pineocytoma |
| choriocarcinoma | pituicytoma |
| choroid plexus papilloma | pituitary adenoma |
| clear-cell sarcoma of the kidney | pituitary tumor |
| craniopharyngioma | plasmacytoma |
| cutaneous T-cell lymphoma | polyembryoma |
| cervical cancer | precursor T-lymphoblastic |
| | lymphoma |
| colorectal cancer | primary central nervous |
| | system lymphoma |
| Degos disease | primary effusion lymphoma |
| desmoplastic small round cell tumor | preimary peritoneal cancer |
| diffuse large B-cell lymphoma | prostate cancer |
| dysembryoplastic neuroepithelial | pancreatic cancer |
| tumor, | |
| dysgerminoma | pharyngeal cancer |
| embryonal carcinoma | pseudomyxoma periotonei |
| endocrine gland neoplasm | renal cell carcinoma |
| endodermal sinus tumor | renal medullary carcinoma |
| enteropathy-associated T-cell | retinoblastoma |
| lymphoma | |
| esophageal cancer | rhabdomyoma |
| fetus in fetu | rhabdomyosarcoma |
| fibroma | Richter's transformation |
| fibrosarcoma | rectal cancer |
| follicular lymphoma | sarcoma |
| follicular thyroid cancer | Schwannomatosis |
| ganglioneuroma | seminoma |
| gastrointestinal cancer | Sertoli cell tumor |
| germ cell tumor | sex cord-gonadal stromal tumor |
| gestational choriocarcinoma | signet ring cell carcinoma |
| giant cell fibroblastoma | skin cancer |
| giant cell tumor of the bone | small blue round cell tumors |
| glial tumor | small cell carcinoma |
| glioblastoma multiforme | soft tissue sarcoma |
| glioma | somatostatinoma |
| gliomatosis cerebri | soot wart |
| glucagonoma | spinal tumor |
| gonadoblastoma | splenic marginal zone lymphoma |
| granulosa cell tumor | squamous cell carcinoma |
| gynandroblastoma | synovial sarcoma |
| gallbladder cancer | Sezary's disease |
| gastric cancer | small intestine cancer |
| hairy cell leukemia | squamous carcinoma |
| hemangioblastoma | stomach cancer |
| head and neck cancer | T-cell lymphoma |
| hemangiopericytoma | testicular cancer |
| hematological malignancy | thecoma |
| hepatoblastoma | thyroid cancer |
| hepatosplenic T-cell lymphoma | transitional cell carcinoma |
| Hodgkin's lymphoma | throat cancer |
| non-Hodgkin's lymphoma | urachal cancer |
| invasive lobular carcinoma | urogenital cancer |
| intestinal cancer | urothelial carcinoma |
| kidney cancer | uveal melanoma |
| laryngeal cancer | uterine cancer |
| lentigo maligna | verrucous carcinoma |
| lethal midline carcinoma | visual pathway glioma |
| leukemia | vulvar cancer |
| leydig cell tumor | vaginal cancer |
| liposarcoma | Waldenstrom's macroglobulinemia |
| lung cancer | Warthin's tumor |
| lymphangioma | Wilms' tumor |
| lymphangiosarcoma | |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia. In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, or papillary thyroid carcinoma.

In another embodiment, the cancer is anaplastic large-cell lymphoma, non-small cell lung cancer, diffuse large B-cell lymphoma, inflammatory myofibroblastic tumors, neuroblastoma, anaplastic thyroid cancer, and rhabdomyosarcoma.

In another embodiment, the cancer is breast cancer, colorectal cancer, esophageal squamous cell cancer, and renal cell carcinoma.

In another embodiment, the cancer is acute leukemia.

In another embodiment, the cancer is carcinomas, including bladder (including accelerated and metastic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma, hematopoietic tumors of myeloid lineage, including acute and chronic myelogenous leukemias, myelodysplastic syndrome, myeloid leukemia, and promyelocytic leukemia; tumors of the central and peripheral nervous system, including astrocytoma, neuroblastoma, glioma, and schwannomas; tumors of mesenchymal origin, including fibrosarcoma, rhabdomyoscarcoma, and osteosarcoma; and other tumors, including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

Additional forms of cancer treatable by a Compound of the Disclosure include, for example, adult and pediatric oncology, growth of solid tumors/malignancies, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma), gastrointestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, and squamous cell cancer), thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. Accordingly, administration of a present cyclic peptidomimetic compound is expected to enhance treatment regimens.

Other cancers that can be treated with the compounds and methods of the invention include, but are not limited to, cancers and metastases selected from the group consisting of solid tumors, including but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiornyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforma, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; blood-borne cancers, including but not limited to: acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myclomonocytic leukemia, acute nonlymphocyctic leukemia, acute undifferentiated leukemia, chronic myclocytic leukemia, chronic lymphocytic leukemia, hairy cell leukemia, and multiple myeloma; acute and chronic leukemias: lymphoblastic, myelogenous lymphocytic, and myelocytic leukemias; lymphomas: Hodgkin's disease and non-Hodgkin's lymphoma; multiple myeloma; Waldenstrom's macroglobulinemia; heavy chain disease; and polycythemia vera.

In another embodiment, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

Compounds of the Disclosure can be used in a variety of settings for the treatment of various cancers. In one embodiment, the individual in need of treatment has previously undergone treatment for cancer. Such previous treatments include, but are not limited to, prior chemotherapy, radiotherapy, surgery, or immunotherapy, such as cancer vaccines.

In another embodiment, the present invention provides a method of treating a cancer comprising: (a) administering to an individual in need thereof a therapeutically effective amount of a Compound of the Disclosure; and (b) administering to the individual an amount of radiotherapy, chemotherapy, or both. The amounts administered are each effective to treat cancer. In another embodiment, the amounts are together effective to treat the cancer.

In another embodiment, the invention provides a method for treating a cancer, said method comprising administering to a subject in need thereof a pharmaceutical composition comprising a therapeutically effective amount of a Compound of the Disclosure to treat the cancer.

In another embodiment, the present disclosure provides therapeutic method of modulating protein methylation, gene expression, cell proliferation, cell differentiation and/or apoptosis in vivo in diseases mentioned above, in particular cancer, inflammatory disease, and/or viral disease is provided by administering a therapeutically effective amount of a Compound of the Disclosure to a subject in need of such therapy.

In another embodiment, the present disclosure provides a method of regulating endogenous or heterologous promoter activity by contacting a cell with a Compound of the Disclosure.

In methods of the present disclosure, a therapeutically effective amount of a Compound of the Disclosure, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the Compound of the Disclosure that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a Compound of the Disclosure can be administered in combination with a second therapeutically active agent. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Non-limiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Non-limiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary non-limiting alkylating agents include cyclophosphamide, ifosfamide, melphalan, and nitrosoureas, such as carmustine and lomustine.

Exemplary non-limiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary non-limiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary non-limiting mTOR inhibitors include compounds that inhibit the mammalian target of rapamycin (mTOR) and possess antiproliferative activity such as sirolimus, everolimus, CCI-779, and ABT578.

Exemplary non-limiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary non-limiting platin compounds include carboplatin, cis-platin, cisplatinum, and oxaliplatin.

Exemplary non-limiting methionine aminopeptidase inhibitors include bengamide or a derivative thereof and PPI-2458.

Exemplary non-limiting bisphosphonates include etridonic acid, clodronic acid, tiludronic acid, pamidronic acid, alendronic acid, ibandronic acid, risedronic acid, and zoledronic acid.

Exemplary non-limiting antiproliferative antibodies include trastuzumab, trastuzumab-DM1, cetuximab, bevacizumab, rituximab, PR064553, and 2C4. The term “antibody” includes intact monoclonal antibodies, polyclonal antibodies, multispecific antibodies formed from at least two intact antibodies, and antibody fragments, so long as they exhibit the desired biological activity.

Exemplary non-limiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary non-limiting telomerase inhibitors include compounds that target, decrease, or inhibit the activity of telomerase, such as compounds that inhibit the telomerase receptor, such as telomestatin.

Exemplary non-limiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary non-limiting Flt-3 inhibitors include PKC412, midostaurin, a staurosporine derivative, SU11248, and MLN518.

Exemplary non-limiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlO1, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cycin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, Cl-1033, EKB-569, GW-2016, antibodies E1.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDCl25, such as okadaic acid or a derivative thereof.

Further anti-angiogenic compounds include compounds having another mechanism for their activity unrelated to protein or lipid kinase inhibition, e.g., thalidomide and TNP-470.

Additional, non-limiting, exemplary chemotherapeutic compounds, one or more of which may be used in combination with a Compound of the Disclosure include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Other examples of second therapeutic agents, one or more of which a Compound of the Disclosure also can be combined, include, but are not limited to: a treatment for Alzheimer's Disease, such as donepezil and rivastigmine; a treatment for Parkinson's Disease, such as L-DOPA/carbidopa, entacapone, ropinrole, pramipexole, bromocriptine, pergolide, trihexephendyl, and amantadine; an agent for treating multiple sclerosis (MS) such as beta interferon (e.g., AVONEX® and REBIF®), glatiramer acetate, and mitoxantrone; a treatment for asthma, such as albuterol and montelukast; an agent for treating schizophrenia, such as zyprexa, risperdal, seroquel, and haloperidol; an anti-inflammatory agent, such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor, such as an acetylcholinesterase inhibitor, an MAO inhibitor, an interferon, an anti-convulsant, an ion channel blocker, riluzole, or an anti-Parkinson's agent; an agent for treating cardiovascular disease, such as a beta-blocker, an ACE inhibitor, a diuretic, a nitrate, a calcium channel blocker, or a statin; an agent for treating liver disease, such as a corticosteroid, cholestyramine, an interferon, and an anti-viral agent; an agent for treating blood disorders, such as a corticosteroid, an anti-leukemic agent, or a growth factor; or an agent for treating immunodeficiency disorders, such as gamma globulin.

The above-mentioned second therapeutically active agents, one or more of which can be used in combination with a Compound of the Disclosure, are prepared and administered as described in the art.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure. In one embodiment, the disclosure provides a pharmaceutical composition comprising a Compound of the Disclosure and a pharmaceutically acceptable excipient.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, e.g., from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, e.g., about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A composition for intravenous, cutaneous, or subcutaneous injection typically contains an isotonic vehicle.

Compounds of the Disclosure can be readily combined with pharmaceutically acceptable carriers well-known in the art. In one embodiment, a pharmaceutical composition comprising a Compound of the Disclosure, or a pharmaceutically acceptable salt or hydrate thereof, and a pharmaceutically acceptable carrier, is provided. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

The term “a disease or condition wherein inhibition of the WDR5-binding partner protein-protein interaction provides a benefit” pertains to a disease or condition in which WDR5 and/or it binding partner, e.g., MLL, is important or necessary, e.g., for the onset, progress, expression of that disease or condition, or a disease or a condition which is known to be treated by an inhibitor of the WDR5-binding partner protein-protein interaction. Examples of such conditions include, but are not limited to, a cancer, a chronic autoimmune disease, an inflammatory disease, a proliferative disease, sepsis, and a viral infection. One of ordinary skill in the art is readily able to determine whether a compound treats a disease or condition mediated by a inhibitor of the WDR5-binding partner protein-protein interaction for any particular cell type, for example, by assays which conveniently can be used to assess the activity of particular compounds.

The term “second therapeutic agent” refers to a therapeutic agent different from a Compound of the Disclosure and that is known to treat the disease or condition of interest. For example when a cancer is the disease or condition of interest, the second therapeutic agent can be a known chemotherapeutic drug, like taxol, or radiation, for example.

The term “disease” or “condition” denotes disturbances and/or anomalies that as a rule are regarded as being pathological conditions or functions, and that can manifest themselves in the form of particular signs, symptoms, and/or malfunctions. As demonstrated below, Compounds of the Disclosure are inhibitors of the WDR5-binding partner protein-protein interaction, e.g., the WDR5-MLL protein-protein interaction, and can be used in treating or preventing diseases and conditions wherein inhibition of the WDR5-binding partner protein-protein interaction provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceuticalproduct.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

In the present disclosure, the term “binding partners” refers to oligomers, polymers, proteins, and related compounds that interact with, e.g., bind, with the arginine binding site of WDR5.

In the present disclosure, the term “halo” as used by itself or as part of another group refers to —Cl, —F, —Br, or —I.

In the present disclosure, the term “nitro” as used by itself or as part of another group refers to —NO₂.

In the present disclosure, the term “cyano” as used by itself or as part of another group refers to —CN.

In the present disclosure, the term “hydroxy” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl is a C₁₋₁₀ alkyl. In another embodiment, the alkyl is a C₁₋₆ alkyl. In another embodiment, the alkyl is a C₁₋₄ alkyl. In another embodiment, the alkyl is a straight chain C₁₋₁₀ alkyl. In another embodiment, the alkyl is a branched chain C₃₋₁₀ alkyl. In another embodiment, the alkyl is a straight chain C₁₋₆ alkyl. In another embodiment, the alkyl is a branched chain C₃₋₆ alkyl. In another embodiment, the alkyl is a straight chain C₁₋₄ alkyl. In another embodiment, the alkyl is a branched chain C₃₋₄ alkyl. In another embodiment, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “optionally substituted alkyl” as used by itself or as part of another group means that the alkyl as defined above is either unsubstituted or substituted with one, two, or three substituents independently selected from the group consisting of nitro, haloalkoxy, aryloxy, aralkyloxy, alkylthio, sulfonamido, alkylcarbonyl, arylcarbonyl, alkylsulfonyl, arylsulfonyl, carboxy, carboxyalkyl, and cycloalkyl. In one embodiment, the optionally substituted alkyl is substituted with two substituents. In another embodiment, the optionally substituted alkyl is substituted with one substituent. Non-limiting exemplary optionally substituted alkyl groups include —CH₂CH₂NO₂, —CH₂SO₂CH₃ CH₂CH₂CO₂H, —CH₂CH₂SO₂CH₃, —CH₂CH₂COPh, and —CH₂C₆H₁₁.

In the present disclosure, the term “cycloalkyl” as used by itself or as part of another group refers to saturated and partially unsaturated (containing one or two double bonds) cyclic aliphatic hydrocarbons containing one, two, or three rings having from three to twelve carbon atoms, i.e., C₃₋₁₂ cycloalkyl, or the number of carbons designated. In one embodiment, the cycloalkyl group has two rings. In one embodiment, the cycloalkyl group has one ring. In another embodiment, the cycloalkyl group is a C₃₋₈ cycloalkyl group. In another embodiment, the cycloalkyl group is a C₃₋₆ cycloalkyl group. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, and cyclohexenyl.

In the present disclosure, the term “optionally substituted cycloalkyl” as used by itself or as part of another group means that the cycloalkyl as defined above is either unsubstituted or substituted with one, two, or three substituents independently selected from the group consisting of from halo, nitro, cyano, hydroxy, amino, haloalkyl, hydroxyalkyl, alkoxy, haloalkoxy, aryloxy, aralkyloxy, alkylthio, amido, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, and (heterocyclo)alkyl. In one embodiment, the optionally substituted cycloalkyl is substituted with two substituents. In another embodiment, the optionally substituted cycloalkyl is substituted with one substituent.

In the present disclosure, the term “alkenyl” as used by itself or as part of another group refers to an alkyl group as defined above containing one, two, or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

In the present disclosure, the term “optionally substituted alkenyl” as used herein by itself or as part of another group means the alkenyl as defined above is either unsubstituted or substituted with one, two or three substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, amido, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, optionally substituted aryl, optionally substituted heteroaryl, and optionally substituted heterocyclo.

In the present disclosure, the term “alkynyl” as used by itself or as part of another group refers to an alkyl group as defined above containing one, two, or three carbon-to-carbon triple bonds. In one embodiment, the alkynyl has one carbon-to-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

In the present disclosure, the term “optionally substituted alkynyl” as used herein by itself or as part of another group means the alkynyl as defined above is either unsubstituted or substituted with one, two or three substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, amido, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, optionally substituted aryl, optionally substituted heteroaryl, and optionally substituted heterocyclo.

In the present disclosure, the term “haloalkyl” as used by itself or as part of another group refers to an alkyl group substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “hydroxyalkyl” as used by itself or as part of another group refers to an alkyl group substituted with one, two, or three, hydroxy groups. In one embodiment, the hydroxyalkyl group is a monohydroxyalkyl group, i.e., substituted with one hydroxy group. In another embodiment, the hydroxyalkyl group is a dihydroxyalkyl group, i.e., substituted with two hydroxy groups.

In another embodiment, the hydroxyalkyl group is a C₁₋₄ hydroxyalkyl group. Non-limiting exemplary hydroxyalkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In one embodiment, the alkoxy group is a C₁₋₄ alkoxy group. In another embodiment, the alkoxy group is a C₁₋₄ alkyl attached to a terminal oxygen atom, e.g., methoxy, ethoxy, and tert-butoxy.

In the present disclosure, the term “alkylthio” as used by itself or as part of another group refers to a sulfur atom substituted by an optionally substituted alkyl group. In one embodiment, the alkylthio group is a C₁₋₄ alkylthio group. Non-limiting exemplary alkylthio groups include —SCH₃ and —SCH₂CH₃.

In the present disclosure, the term “alkoxyalkyl” as used by itself or as part of another group refers to an alkyl group substituted with an alkoxy group. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

In the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic or bicyclic aromatic ring system having from six to fourteen carbon atoms, i.e., C₆-C₁₄ aryl, or the number of carbon atoms designated. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

In the present disclosure, the term “optionally substituted aryl” as used herein by itself or as part of another group means that the aryl as defined above is either unsubstituted or substituted with one to five substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, amido, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, and (heterocyclo)alkyl.

In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, and 3-chloro-4-fluorophenyl. The term optionally substituted aryl is meant to include groups having fused optionally substituted cycloalkyl and fused optionally substituted heterocyclo rings. Non-limiting examples include:

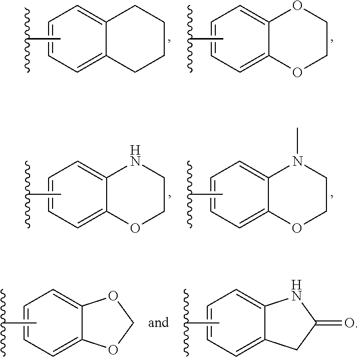

In the present disclosure, the term “aryloxy” as used by itself or as part of another group refers to an optionally substituted aryl attached to a terminal oxygen atom. A non-limiting exemplary aryloxy group is PhO—.

In the present disclosure, the term “aralkyloxy” as used by itself or as part of another group refers to an aralkyl group attached to a terminal oxygen atom. A non-limiting exemplary aralkyloxy group is PhCH₂O—.

In the present disclosure, the term “heteroaryl” or “heteroaromatic” refers to monocyclic and bicyclic aromatic ring systems having 5 to 14 ring atoms, i.e., a 5- to 14-membered heteroaryl, wherein at least one carbon atom of one of the rings is replaced with a heteroatom independently selected from the group consisting of oxygen, nitrogen and sulfur. In one embodiment, the heteroaryl contains 1, 2, 3, or 4 heteroatoms independently selected from the group consisting of oxygen, nitrogen and sulfur. In one embodiment, the heteroaryl has three heteroatoms. In another embodiment, the heteroaryl has two heteroatoms. In another embodiment, the heteroaryl has one heteroatom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), or indazolyl (e.g., 1H-indazol-3-yl). The term “heteroaryl” is also meant to include possible N-oxides. A non-limiting exemplary N-oxide is pyridyl N-oxide. The heteroaryl can be attached to the remained of the molecule through any available carbon or nitrogen atom.

In one embodiment, the heteroaryl is a 5- or 6-membered heteroaryl. In one embodiment, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl.

In another embodiment, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6-membered heteroaryl groups include pyridyl, pyrazinyl, pyrimidinyl, and pyridazinyl.

In the present disclosure, the term “optionally substituted heteroaryl” as used by itself or as part of another group means that the heteroaryl as defined above is either unsubstituted or substituted with one to four substituents, e.g., one or two substituents, independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, amido, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, and (heterocyclo)alkyl. In one embodiment, the optionally substituted heteroaryl has one substituent. Any available carbon or nitrogen atom can be substituted.

The term optionally substituted heteroaryl is also meant to include groups having fused optionally substituted cycloalkyl and fused optionally substituted heterocyclo rings. Non-limiting examples include:

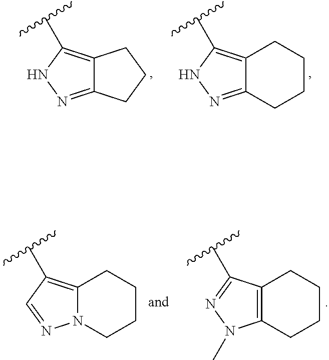 [SMILES: *c1[nH]nc2c1CCC2 ; *c1[nH]nc2c1CCCC2 ; *c1cnn2c1CCCC2 ; *c1nn(C)c2c1CCCC2]

In another embodiment, the heteroaryl is an optionally substituted 9- to 14-membered bicyclic aromatic ring system, wherein at least one carbon atom of one of the rings is replaced with a heteroatom independently selected from the group consisting of oxygen, nitrogen, and sulfur. Non-limiting exemplary 9- to 14-membered bicyclic aromatic ring systems include:

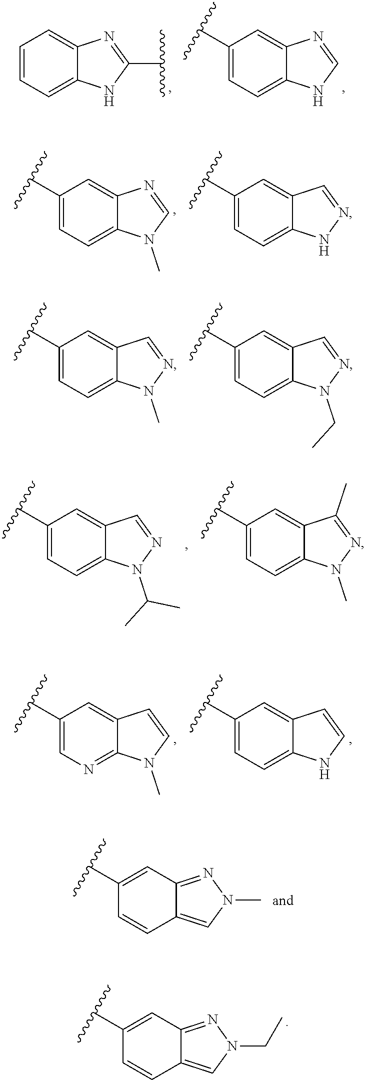

In the present disclosure, the term “heterocycle” or “heterocyclo” as used by itself or as part of another group refers to saturated and partially unsaturated (e.g., containing one or two double bonds) cyclic groups containing one, two, or three rings having from three to fourteen ring members, i.e., a 3- to 14-membered heterocyclo, wherein at least one carbon atom of one of the rings is replaced with a heteroatom. In one embodiment, the heterocyclo is a 4- to 8-membered heterocyclo. Each heteroatom is independently selected from the group consisting of oxygen, sulfur, including sulfoxide and sulfone, and/or nitrogen atoms, which can be oxidized or quaternized. The term “heterocyclo” is meant to include groups wherein a ring —CH₂— is replaced with a —C(═O)—, for example, cyclic ureido groups such as 2-imidazolidinone and cyclic amide groups such as β-lactam, γ-lactam, δ-lactam, ε-lactam, and piperazin-2-one. The term “heterocyclo” is also meant to include groups having fused optionally substituted aryl groups, e.g., indolinyl, chroman-4-yl. In one embodiment, the heterocyclo group is a 5- or 6-membered cyclic group containing one ring and one or two oxygen and/or nitrogen atoms. The heterocyclo can be optionally linked to the rest of the molecule through any available carbon or nitrogen atom. Non-limiting exemplary heterocyclo groups include dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl.

In the present disclosure, the term “optionally substituted heterocyclo” as used herein by itself or part of another group means the heterocyclo as defined above is either unsubstituted or substituted with one to four substituents independently selected from halo, nitro, cyano, hydroxy, amino, alkylamino, dialkylamino, haloalkyl, hydroxyalkyl, alkoxy, haloalkoxy, aryloxy, aralkyloxy, alkylthio, amido, carboxamido, sulfonamido, alkylcarbonyl, alkoxycarbonyl, CF₃C(═O)—, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. Substitution may occur on any available carbon or nitrogen atom, or both. Non-limiting exemplary optionally substituted heterocyclo groups include:

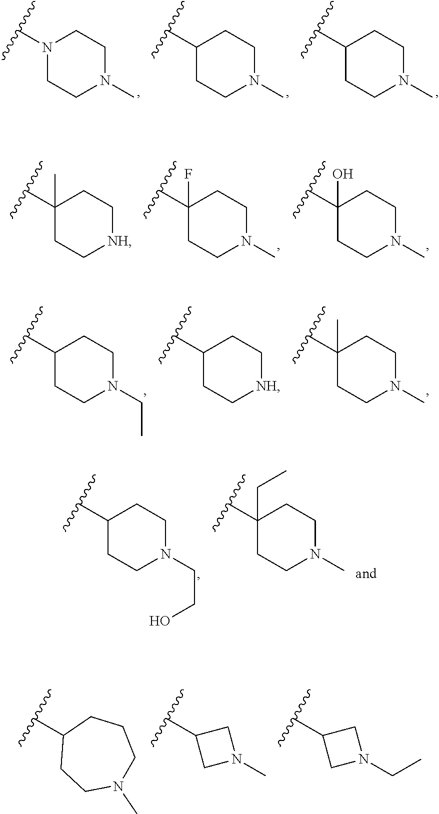

In the present disclosure, the term “amino” as used by itself or as part of another group refers to a radical of the formula —NR^{30a}R^{30b}, wherein R^{30a} and R^{30b} are independently hydrogen, alkyl, hydroxyalkyl, optionally substituted cycloalkyl, optionally substituted aryl, optionally substituted heterocyclo, or optionally substituted heteroaryl, or R^{30a} and R^{30b} a taken together to form a 3- to 8-membered optionally substituted heterocyclo. In one embodiment, R^{30a} and R^{30b} are independently hydrogen or C₁₋₄ alkyl. Non-limiting exemplary amino groups include —NH₂ and —N(H)(CH₃).

In the present disclosure, the term “(amino)alkyl” as used by itself or as part of another group refers to an alkyl group substituted with an amino group. In one embodiment, the (amino)alkyl is a C₁₋₆ alkyl substituted with an amino group, i.e., an (amino)C₁₋₆ alkyl. In another embodiment, the (amino)alkyl is an (amino)C₁₋₄ alkyl. Non-limiting exemplary (amino)alkyl groups include —CH₂CH₂NH₂, —CH₂CH₂N(H)CH₃, —CH₂CH₂N(CH₃)₂, and —CH₂N(H)cyclopropyl.

In the present disclosure, the term “carboxamido” as used by itself or as part of another group refers to a radical of formula —C(═O)NR^{31a}R^{31b}, wherein R^{31a} and R^{31b} are each independently hydrogen, optionally substituted alkyl, hydroxyalkyl, optionally substituted cycloalkyl, optionally substituted aryl, optionally substituted heterocyclo, or optionally substituted heteroaryl, or R^{31a} and R^{31b} taken together with the nitrogen to which they are attached form a 3- to 8-membered optionally substituted heterocyclo group. In one embodiment, R^{31a} and R^{31b} are each independently hydrogen or optionally substituted alkyl. In one embodiment, R^{31a} and R^{31b} are taken together to taken together with the nitrogen to which they are attached form a 3- to 8-membered optionally substituted heterocyclo group. Non-limiting exemplary carboxamido groups include —CONH₂, —CON(H)CH₃, —CON(CH₃)₂, and —CON(H)Ph.

In the present disclosure, the term “amido” as used by itself or as part of another group refers to a radical of formula —N(R^{32a})C(═O)R^{32b}, wherein R^{32a} is hydrogen or C₁₋₄ alkyl; and R^{32b} is C₁₋₆ alkyl, optionally substituted cycloalkyl, optionally substituted aryl, C₁₋₄ alkoxy, or amino. In one embodiment, R^{32a} is hydrogen. In another embodiment, R^{32b} is C₁₋₄ alkyl, C₁₋₄ alkoxy, or amino. Non-limiting exemplary amido groups include —N(H)C(═O)CH₃, —N(H)C(═O)OCH₃, and —N(H)C(═O)N(H)CH₃.

In the present disclosure, the term “sulfonamido” as used by itself or as part of another group refers to a radical of the formula —SO₂NR^{8a}R^{8b}, wherein R^{8a} and R^{8b} are each independently hydrogen, optionally substituted alkyl, or optionally substituted aryl, or R^{8a} and R^{8b} taken together with the nitrogen to which they are attached from a 3- to 8-membered heterocyclo group. Non-limiting exemplary sulfonamido groups include —SO₂NH₂, —SO₂N(H)CH₃, and —SO₂N(H)Ph.

In the present disclosure, the term “alkylcarbonyl” as used by itself or as part of another group refers to a carbonyl group, i.e., —C(═O)—, substituted by an alkyl group. A non-limiting exemplary alkylcarbonyl group is —COCH₃.

In the present disclosure, the term “arylcarbonyl” as used by itself or as part of another group refers to a carbonyl group, i.e., —C(═O)—, substituted by an optionally substituted aryl group. A non-limiting exemplary arylcarbonyl group is —COPh.

In the present disclosure, the term “alkoxycarbonyl” as used by itself or as part of another group refers to a carbonyl group, i.e., —C(═O)—, substituted by an alkoxy group. Non-limiting exemplary alkoxycarbonyl groups include —C(═O)OMe, —C(═O)OEt, and —C(═O)OtBu.

In the present disclosure, the term “alkylsulfonyl” as used by itself or as part of another group refers to a sulfonyl group, i.e., —SO₂—, substituted by any of the above-mentioned optionally substituted alkyl groups. A non-limiting exemplary alkylsulfonyl group is —SO₂CH₃.

In the present disclosure, the term “arylsulfonyl” as used by itself or as part of another group refers to a sulfonyl group, i.e., —SO₂—, substituted by any of the above-mentioned optionally substituted aryl groups. A non-limiting exemplary arylsulfonyl group is —SO₂Ph.

In the present disclosure, the term “mercaptoalkyl” as used by itself or as part of another group refers to any of the above-mentioned alkyl groups substituted by a —SH group.

In the present disclosure, the term “carboxy” as used by itself or as part of another group refers to a radical of the formula —COOH.

In the present disclosure, the term “carboxyalkyl” as used by itself or as part of another group refers to any of the above-mentioned alkyl groups substituted with a —COOH. A non-limiting exemplary carboxyalkyl group is —CH₂CO₂H.

In the present disclosure, the terms “aralkyl” or “arylalkyl” as used by themselves or as part of another group refers to an alkyl group substituted with one, two, or three optionally substituted aryl groups. In one embodiment, the optionally substituted aralkyl group is a C₁₋₄ alkyl substituted with one optionally substituted aryl group. In one embodiment, the optionally substituted aralkyl group is a C₁ or C₂ alkyl substituted with one optionally substituted aryl group. In one embodiment, the optionally substituted aralkyl group is a C₁ or C₂ alkyl substituted with one optionally substituted phenyl group. Non-limiting exemplary optionally substituted aralkyl groups include benzyl, phenethyl, —CHPh₂, —CH₂(4-F-Ph), —CH—₂(4-Me-Ph), —CH₂(4-CF₃-Ph), and —CH(4-F-Ph)₂.

In the present disclosure, the term “(cycloalkyl)alkyl,” as used by itself or as part of another group refers to an alkyl substituted with an optionally substituted cycloalkyl. In one embodiment, the (cycloalkyl)alkyl, is a “(C₃₋₆ cycloalkyl)C₁₋₄ alkyl,” i.e., a C₁₋₄ alkyl substituted with an optionally substituted C₃₋₆ cycloalkyl. Non-limiting exemplary (cycloalkyl)alkyl groups include:

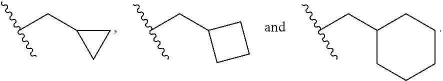

In the present disclosure, the terms “(heterocyclo)alkyl” as used by itself or part of another group refers to an alkyl group substituted with one or two optionally substituted heterocyclo groups. In one embodiment, the (heterocyclo)alkyl is a C₁₋₄ alkyl substituted with one optionally substituted heterocyclo group, i.e., a (heterocyclo)C₁₋₄ alkyl. In another embodiment, the (heterocyclo)alkyl is a C₁₋₄ alkyl substituted with one optionally substituted 4- to 8-membered heterocyclo group, i.e., a (4- to 8-membered heterocyclo)C₁₋₄ alkyl. Non-limiting exemplary (heterocyclo)alkyl groups include:

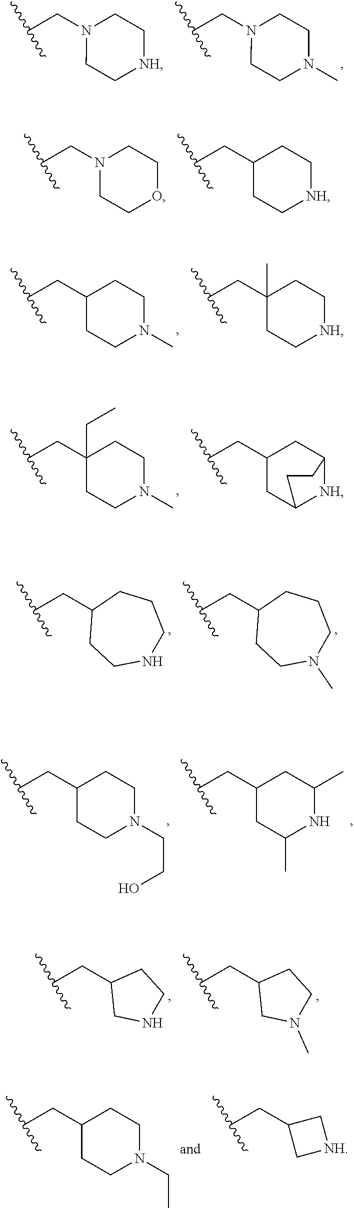

In the present disclosure, the terms “(heteroaryl)alkyl” as used by itself or part of another group refers to an alkyl group substituted with one or two optionally substituted heteroaryl groups. In one embodiment, the (heteroaryl)alkyl is a C₁₋₄ alkyl substituted with one optionally substituted heteroaryl group, i.e., a (heteroaryl)C₁₋₄ alkyl. In another embodiment, the (heteroaryl)alkyl is a C₁₋₄ alkyl substituted with one optionally substituted 5- or 6-membered heteroaryl group, i.e., a (5- or 6-membered heteroaryl)C₁₋₄ alkyl. Non-limiting exemplary (heteroaryl)alkyl groups include:

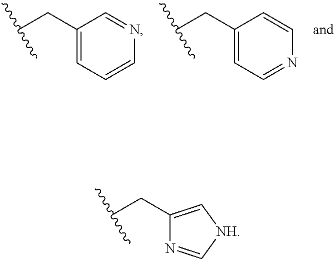

### EXAMPLES

### General

All the final compounds were characterized with H-NMR, 1³C-NMR (300 MHz or 400 MHz, Bruker), and/or HRMS (ESI+) (Agilent Q-TOF Electrospray). The intermediates were characterized with ¹H-NMR, 1³C-NMR (300 MHz or 400 MHz, Bruker) and/or MS (ESI+) (Thermo Scientific LCQ Fleet). Chemical shifts were reported in ppm relative to TMS. D₂O (4.79 ppm), CD₃OD (3.31 ppm), CD₃CN (1.94 ppm) or DMSO-d₆ (2.50 ppm) was used as the internal standard for H-NMR spectra. D₂O (1,4-dioxane, 66.7 ppm), CD₃OD (49.2 ppm), CD₃CN (1.4 ppm) or DMSO-d₆ (39.5 ppm) was used as internal standard for 1³C-NMR spectra. The final products were purified on a preparative HPLC (Waters 2545, Quaternary Gradient Module) with a SunFire Prep C18 OBD 5 m 50×100 mm reverse-phase column. The mobile phase was a gradient of solvent A (0.1% Trifluoroacetic acid in water) and solvent B (0.1% Trifluoroacetic acid in CH₃CN) at a flow rate of 60 mL/min and 1%/1 min increase of solvent B. All final compounds have purity≥95% as determined by Waters ACQUITY UPLC using reverse-phase column (SunFire, C18, 5 μm, 4.6×150 mm) and a solvent gradient of A (0.1% of Trifluoroacetic acid in water) and solvent B (0.1% of Trifluoroacetic acid in CH₃CN). The final compounds were isolated and characterized as 2,2,2-trifluoroacetate (TFA) salts unless otherwise indicated.

### Example 1

### Competitive Binding Experiments to WDR5 Protein

Binding affinities were tested using a fluorescence polarization (FP) based competitive binding assay described earlier (Karatas et al., J. Med. Chem. 53:5179-5185 (2010)). Briefly, to a 5 μl solution of the tested compound in DMSO, 120 μl of pre-incubated complex solution (N-terminal His-tagged WDR5 protein (residues 24-334), named WDR5Δ23, and 5-FAM labeled tracer) in assay buffer (0.1M Phosphate, 25 mM KCl, 0.01% Triton, pH 6.5) was added, giving final concentrations of WDR5Δ23 and the tracer to be 4 nM and 0.6 nM, respectively. The plates were incubated at room temperature on a shaker for 3h, and then the mP values were measured using the Tecan Infinite M-1000 plate reader (Tecan U.S., Research Triangle Park, N.C.). Ki values were calculated using the equation described previously (Nikolovska-Coleska Z., et al., Anal. Biochem, 332:261-273 (2004)). See Table 2.

TABLE 2

| | | | | Inhibition of | | | |
|---|---|---|---|---|---|---|---|
| | | Binding Affinity to | | MLL HMT | Cell Growth Inhibition | | |
| | | WDR5 | | Activity | IC₅₀ (μM) | | |
| Cpd. No. | Structure | IC₅₀ ± SD (nM) | Kᵢ ± SD (nM) | IC₅₀ ± SD (nM) | MOLM-13 | MV-4-11 | HL-60 |
| 1 | 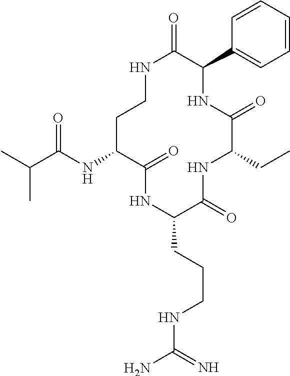 [SMILES: CC[C@@H]1NC(=O)[C@H](CCCNC(=N)N)NC(=O)[C@H](NC(=O)C(C)C)CCNC(=O)[C@@H](c2ccccc2)NC1=O] | 6.2 ± 0.5 | 0.8 ± 0.1 | 1.8 ± 0.4 | 42.8 | 43.4 | >100 |
| 4 | 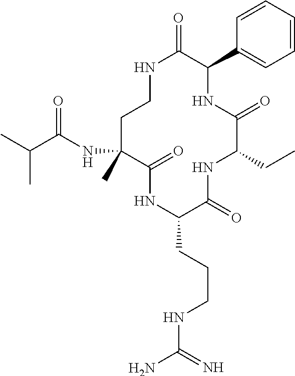 [SMILES: CC[C@@H]1NC(=O)[C@H](CCCNC(=N)N)NC(=O)[C@](C)(NC(=O)C(C)C)CCNC(=O)[C@@H](c2ccccc2)NC1=O] | 1.08 ± 0.12 | <1 | 12.6 ± 1.2 | 0.28 ± 0.02 | 0.46 ± 0.02 | 17.4 |
| 5 | 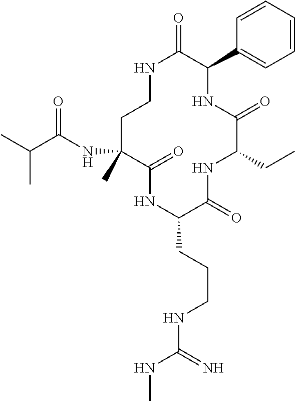 [SMILES: CC[C@@H]1NC(=O)[C@H](CCCNC(=N)NC)NC(=O)[C@](C)(NC(=O)C(C)C)CCNC(=O)[C@@H](c2ccccc2)NC1=O] | 0.90 ± 0.20 | <1 | 12.7 ± 1.5 | 0.21 ± 0.02 | 0.25 ± 0.01 | 8.56 ± 1.14 |
| 62 | 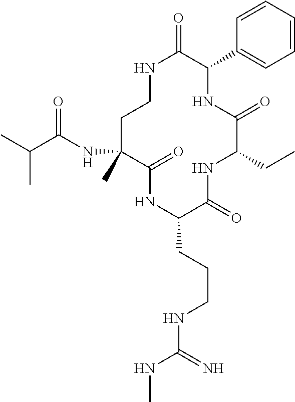 [SMILES: CC[C@@H]1NC(=O)[C@H](CCCNC(=N)NC)NC(=O)[C@](C)(NC(=O)C(C)C)CCNC(=O)[C@H](c2ccccc2)NC1=O] | 2.0 ± 0.1 | <1 | 477 ± 51 | 14.3 ± 0.9 | 35.0 ± 4.4 | Not tested |
| 63 | 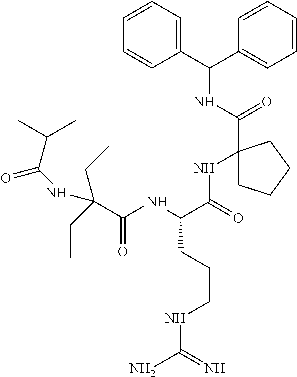 [SMILES: CCC(CC)(NC(=O)C(C)C)C(=O)N[C@@H](CCCNC(=N)N)C(=O)NC1(C(=O)NC(c2ccccc2)c2ccccc2)CCCC1] | 2.9 ± 1.4 | <1 | 578 ± 190 | | | |
| 64 | 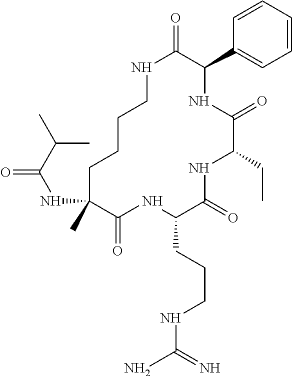 [SMILES: CC[C@@H]1NC(=O)[C@H](CCCNC(=N)N)NC(=O)[C@](C)(NC(=O)C(C)C)CCCCNC(=O)[C@@H](c2ccccc2)NC1=O] | 0.9 ± 0.2 | <1 | 373 ± 44 | 18.4 ± 6.6 | 9.8 ± 8.0 | >100 |
| 65 | 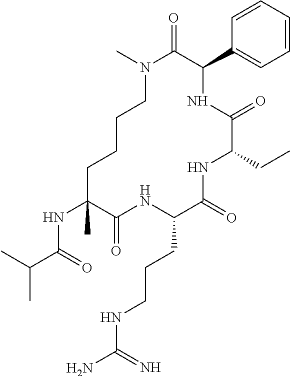 [SMILES: CC[C@@H]1NC(=O)[C@H](CCCNC(=N)N)NC(=O)[C@](C)(NC(=O)C(C)C)CCCCN(C)C(=O)[C@@H](c2ccccc2)NC1=O] | 1.6 ± 0.3 | <1 | 190 ± 33 | | | |
| 66 | 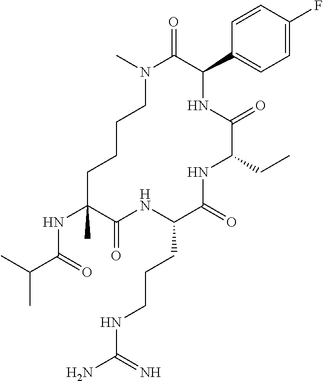 [SMILES: CC[C@@H]1NC(=O)[C@H](CCCNC(=N)N)NC(=O)[C@](C)(NC(=O)C(C)C)CCCCN(C)C(=O)[C@@H](c2ccc(F)cc2)NC1=O] | 1.6 ± 1.0 | <1 | 138 ± 39 | 16.2 ± 6.1 | 5.2 ± 1.1 | 60.0 ± 19.7 |
| 67 | 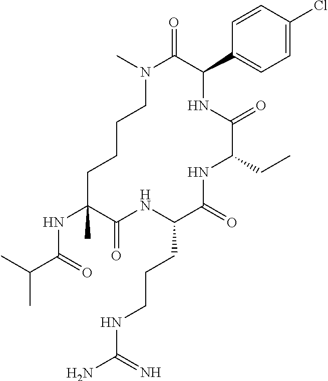 [SMILES: CC[C@@H]1NC(=O)[C@H](CCCNC(=N)N)NC(=O)[C@](C)(NC(=O)C(C)C)CCCCN(C)C(=O)[C@@H](c2ccc(Cl)cc2)NC1=O] | 1.5 ± 0.9 | <1 | 124 ± 28 | | | |
| 68 | 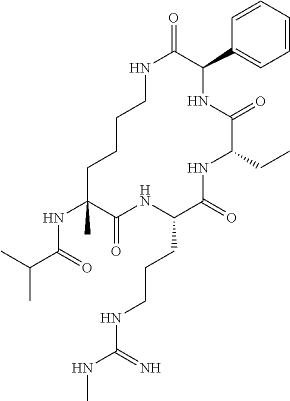 [SMILES: CC[C@@H]1NC(=O)[C@H](CCCNC(=N)NC)NC(=O)[C@](C)(NC(=O)C(C)C)CCCCNC(=O)[C@@H](c2ccccc2)NC1=O] | 1.1 ± 0.3 | <1 | 452 ± 55 | 3.6 ± 0.8 | 4.3 ± 1.5 | >100 |
| 69 | 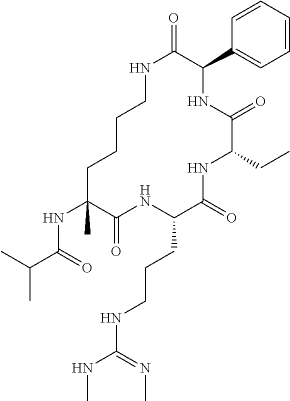 [SMILES: CC[C@@H]1NC(=O)[C@H](CCCN/C(=N/C)NC)NC(=O)[C@](C)(NC(=O)C(C)C)CCCCNC(=O)[C@@H](c2ccccc2)NC1=O] | 2.4 ± 0.7 | <1 | 1259 ± 236 | 31.5 ± 9.8 | 15.2 ± 2.9 | 84.8 ± 13.2 |
| 70 | 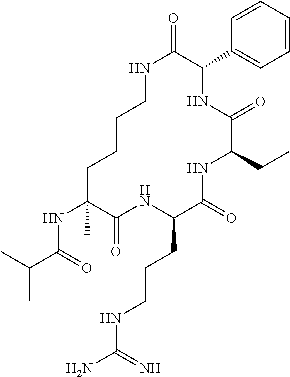 [SMILES: CC[C@H]1NC(=O)[C@@H](CCCNC(=N)N)NC(=O)[C@@](C)(NC(=O)C(C)C)CCCCNC(=O)[C@H](c2ccccc2)NC1=O] | >10,000 | | >10,000 | >100 | >100 | >100 |
| 71 | 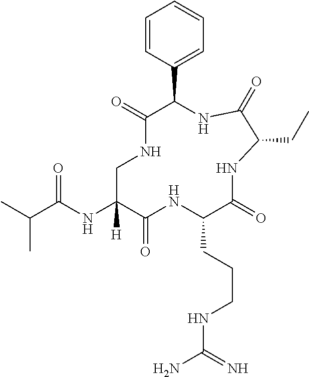 [SMILES: CC[C@@H]1NC(=O)[C@H](CCCNC(=N)N)NC(=O)[C@H](NC(=O)C(C)C)CNC(=O)[C@@H](c2ccccc2)NC1=O] | 1190 ± 160 | 242 ± 33 | >10 | | | |
| 72 | 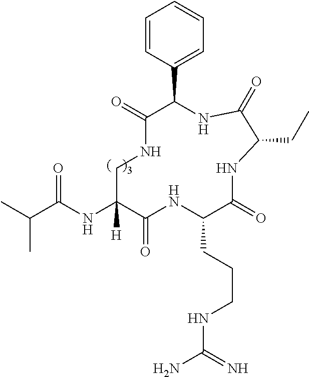 | 164 ± 26 | 32.9 ± 5.3 | >10 | | | |
| 73 | 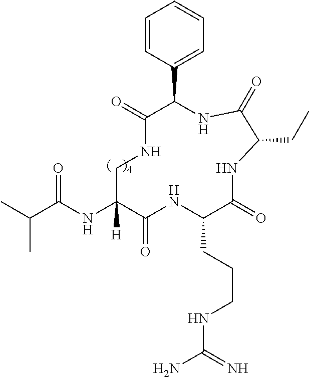 | 298 ± 58 | 60.3 ± 11.9 | >10 | | | |
| 74 | 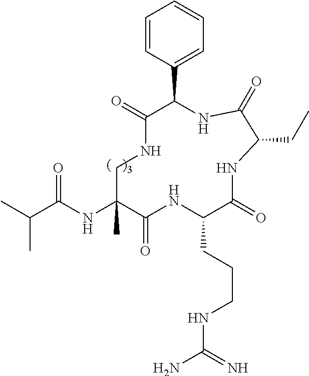 | 0.97 ± 0.13 | <1 | 84.8 ± 23.8 | 1.28 ± 0.01 | 1.29 | 46.8 |
| 75 | 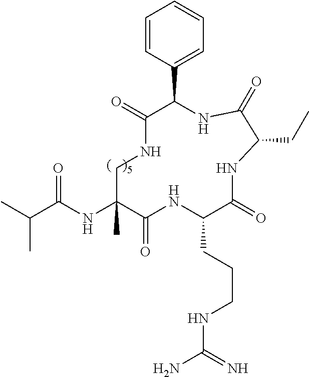 | 2.6 ± 0.2 | <1 | 1527 ± 218 | 37.6 ± 12.1 | 15.1 ± 11.8 | >100 |
| 76 | 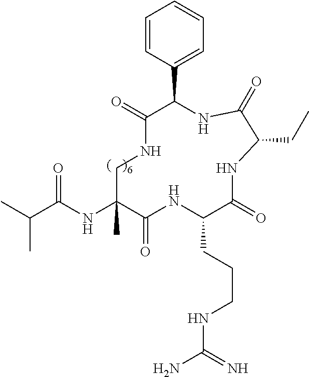 | 1.0 ± 0.2 | <1 | 216 ± 88 | 5.6 ± 0.7 | 3.2 ± 0.4 | 29.7 ± 11.5 |
| 77 | 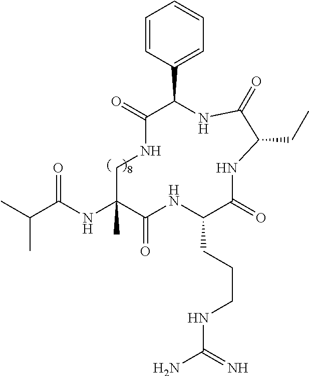 | 1.3 ± 0.2 | <1 | 744 ± 79 | 21.6 ± 4.8 | 7.5 ± 3.0 | >100 |

### Example 2

### In Vitro Cell-Free MLL HMT Functional Assay

Because the WDR5-MLL interaction is required for the MLL complex to achieve robust H3-K4 HMT activity, compounds that target this interaction are predicted to effectively inhibit the MLL H3-K4 HMT activity. Existing MLL HMT functional assays utilize radioactive ³H labeled S-Adenosyl methionine (SAM) and thus involve multiple wash and transfer steps (Karatas et al., J. Med. Chem. 53:5179-5185 (2010)).

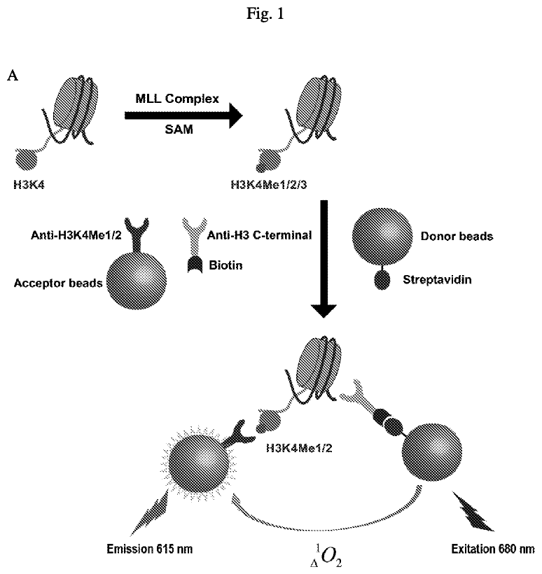

To avoid multiple wash and transfer steps, an amplified luminescent proximity homogeneous assay (AlphaLISA) was developed and optimized to evaluate the MLL HMT inhibitory activities of the Compounds of the Disclosure. The assay flow is shown in FIG. 1.

In this assay, recombinant nucleosomes are initially treated with the MLL complex for methylation in the presence of S-Adenosyl methionine (SAM) as cofactor before stopping the reaction with high salt buffer. Then, anti-H3K4Mel/2 antibody, which is covalently linked to acceptor beads, and biotinylated anti-H3 (C-terminus) antibody were added. Lastly, streptavidin labeled donor beads were added, and the assay plate was imaged with a microplate reader using excitation wavelength of 680 nm and emission wavelength of 615 nm.

Different from widely used Homogeneous Time Resolved Fluorescence (HTRF) assays, in which energy transfer can only take place between donor-acceptor fluorophores located within approximate 10 nm, short-lived singlet oxygen (¹O₂) generated from AlphaLISA donor beads can reach the acceptor beads as far as 200 nm away. This extra-long effective range significantly enhances the versatility of Alpha assays, in which multiple labeled antibodies can be utilized. Such feature is also particularly useful for HMT functional assays involving large-size components, such as nucleosomes as substrates, which are 11 nm in size (e.g. mononucleosomes) or much larger (e.g. oligonucleosomes). Histone methylation assays that use nucleosomes as substrates more closely mimic the MLL HMT reaction in cells, compared with those that utilize histone peptide or proteins as substrates. Therefore, a functional AlphaLISA MLL HMT assay using nucleosomes as substrates would be useful to evaluate these WDR5 inhibitors.

Although no robust MLL HMT AlphaLISA assay has been reported, the acceptor bead conjugated with antibodies which can recognize methylated H3K4 is commercially available and was used here to develop the MLL HMT AlphaLISA assay. As indicated by the manufacturer and experimentally confirmed, this antibody can only recognize mono- and di-methylated H3K4. The assay conditions were optiminzed to minimize tri-methylation of nucleosomes which would greatly compromise the interactions of acceptor beads with mono- and di-methylated nucleosomes and thereby significantly reduce fluorescence intensity detected in the assay yielding a low dynamic range.

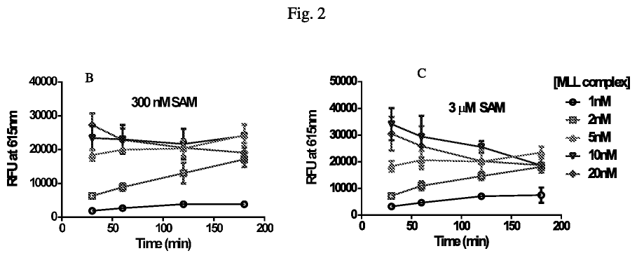

The influence of reaction time and concentration of the MLL complex on fluorescence intensity using two different SAM concentrations (FIG. 2) was investigated. At low SAM concentration (300 nM), AlphaLISA fluorescence signals increased with longer reaction time when MLL complex concentration was 1-5 nM, kept steady with 10 nM MLL complex, but decreased as early as in 30 minutes with 20 nM of the MLL complex. When SAM concentration was 10-fold higher (3 μM) with which reaction was even faster, only a slight signal increase was observed when using 5 nM of the MLL complex and significant signal decreasing started at 30 minutes with both 10 and 20 nM of the MLL complex. AlphaLISA signal decrease was observed with higher MLL complex concentrations, longer reaction time, and even more severely with higher SAM concentrations, which is most likely attributed to generation of more tri-methylated nucleosomes.

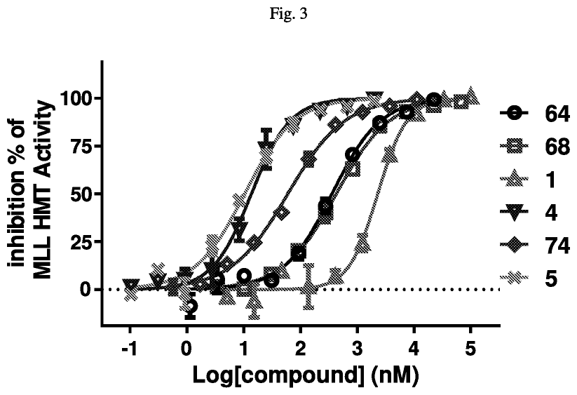

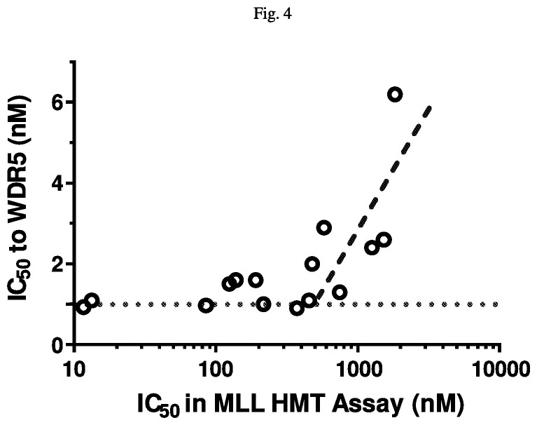

Inhibitory curves of representative WDR5 inhibitors using the optimized AlphaLISA MLL HMT functional assay are shown in FIG. 3. This new functional assay was able to rank compounds with very high affinities to WDR5 as determined in the FP-based competitive binding assay. The IC₅₀ values obtained from the FP-based competitive binding assay was compared with those obtained from the AlphaLISA functional assay for all the compounds included in the present study (FIG. 4). The data showed that there is a linear correlation between the IC₅₀ values obtained from WDR5 competitive binding assay (y-axis) and MLL HMT functional assay (x-axis) as shown with dashed line. However, compounds with IC₅₀ values of 1-2 nM in the WDR5 competitive binding assay deviate from this linear correlation and have IC₅₀ values ranging between 10 and 500 nM in the functional assay. This data indicates that the MLL HMT functional assay can discriminate highly potent WDR5 inhibitors, which is not possible with the competitive binding assay.

In terms of the specific MLL HMT AlphaLISA assay conditions, recombinant MLL complex containing human MLL (MLL1) protein (3735-3973) with N-terminal GST tag and MW=53.7 kDa; full length human WDR5 with N-terminal 6×His tag and MW=35 kDa; full length human ASH2L with N-terminal 6×His tag and MW=61 kDa; full length human RbBP5 with N-terminal 6×His-tag and MW=60 kDa, and full length human DPY30 with N-terminal 6×His-tag and MW=12 kDa, and recombinant nucleosomes were obtained from Activmotif (Carlsbad, Calif.). Anti-Histone H3 Lysine 4 (H3K4mel-2) AlphaLISA acceptor beads, AlphaScreen Streptavidin donor beads and biotinylated anti-H3 (C-terminus) antibody were obtained from PerkinElmer Life Sciences (Waltham, Mass.). 2.5 μl of compound serial dilutions in assay buffer with 4% DMSO and 5 μl of pentameric MLL complex solution were added into a white low volume 384 well microtiter plate which was incubated for 30 minutes with gentle shaking at room temperature, followed by adding 2.5 μl of SAM/Nucleosome mixture. The methylation reaction was performed in 50 mM Tris, pH 8.5 with 1 mM DTT and 0.01% Tween-20 added right before the assay. Final concentrations of MLL complex, SAM, and nucleosomes were 5 nM, 200 nM, and 3 nM, respectively. Final DMSO in the reaction mixture was 1%. The reaction was allowed to perform for 120 minutes in dark with gentle shaking at room temperature. Concentrations of reaction components and times were adjusted accordingly for assay development experiments. 5 μL of high salt stopping solution (50 mM Tris, pH 7.4 with 1 M NaCl, 0.1% Tween-20, and 0.3% poly-L-Lysine) was added to stop the methylation reactions for 15 minutes. 5 μl of 5× acceptor beads/biotinylated anti-H3 antibody mixture in detection buffer (50 mM Tris, pH 7.4 with 0.3 M NaCl, 0.1% Tween-20, and 0.001% poly-L-Lysine) was added, followed by 1 hour incubation at room temperature to allow full interaction between antibodies and methylated nucleosomes. Add 5 μL of 5× streptavidin donor beads in detection and incubate 30 minutes.

Plates were read on a BMG CLARIOstar microplate reader with an excitation wavelength of 680 nm and emission wavelength of 615 nm. IC₅₀ values of compounds were obtained by fitting the fluorescence intensities detected at 615 nm vs compound concentrations in a sigmoidal dose-response curve (variable slope) with a non-linear regression, using Graphpad Prism 6.0 software (Graphpad Software, San Diego, Calif.). See Table 2 and Table 3A.

TABLE 3A

| | Binding Affinity to WDR5 | |
|---|---|---|
| Cpd. No. | IC₅₀ (nM) | Kᵢ (nM) |
| 53 | 0.8783 | <1 |
| 84 | 1.478 | <1 |
| 5 | 2.129 | <1 |
| 12 | 3.253 | <1 |
| 17 | 2.397 | <1 |
| 88 | 2.378 | <1 |
| 89 | 1.833 | <1 |
| 91 | 3.649 | <1 |
| 80 | 2.874 | <1 |
| 82 | 2.302 | <1 |
| 79 | 0.3195 | <1 |
| 92 | 1.865 | <1 |
| 93 | 1.794 | <1 |
| 94 | 3.997 | <1 |
| 98 | 2.542 | <1 |

The specificity of Cpd. No. 5 was tested against MLL and other SET1 family members (MLL2, MLL3, MLL4, SET1a and SET1b) using the previously published HMT assays (Karatas et al., J. Med. Chem. 53:5179-5185 (2010)). Cpd. No. 5 effectively inhibits MLL HMT activity (IC₅₀=12.7 nM), it has no or a minimal effect up to 100 μM in inhibition of the HMT activity of other SET1 family members (data not shown).

### Example 3

### Purification of WDR5 Protein

WDR5 (residues 24-334) was cloned into a His₆-SUMO-vector. The protein was expressed in E. coli. Rosetta™ 2 (DE3) cells using Luria Broth media. Cells were subsequently sonicated in 25 mM Tris pH 8.0, 500 mM NaCl, 5% glycerol, 1 mM benzamidine, 0.1% NP40 and 0.1% ME with protease inhibitors. The cellular debris was pelleted at 17,000 rpm for 45 min and the supernatant was loaded onto a Ni-NTA resin (Qiagen) pre-equilibrated with 25 mM Tris pH 8.0, 500 mM NaCl, 20 mM imidazole and 5% glycerol. The column was then washed with 25 mM Tris pH 8.0, 1 M NaCl and 5% glycerol to remove contaminants and the protein eluded with 25 mM Tris pH 8.0, 150 mM NaCl, 250 mM imidazole and 5% glycerol. The elute was incubated with His₆-Ulp1 and dialyzed against 25 mM Tris pH 8.0 and 150 mM NaCl overnight at 4° C., then applied to fresh Ni-NTA to remove the cleaved tag and protease. The flow through of the Ni-NTA column was loaded onto a Source S column (GE Healthcare) pre-equilibrated with 25 mM Tris pH 8.0 and 5% glycerol. The protein was eluded with a 0-500 mM NaCl gradient. For crystallographic studies, the protein was then dialyzed against 25 mM Tris pH 8.0 and 150 mM NaCl overnight at 4° C., concentrated to 20-30 mg/mL and stored at −80° C.

### Example 4

### Crystallization and Structural Determination

WDR5/Cpd. No. 76 binary complex was obtained by mixing WDR5 and Cpd. No. 76 at molar ratio 1:2 before crystallization. The complex was crystallized in 25% tert-butanol, 0.1M Tris-HCl, pH7.5 at 293K. The crystals were harvested in the same buffer with 20% glycerol. The 2.8 Å dataset was collected at Advanced Photon Source beamline 21ID-G and was processed by HKL2000 (McCoy et al., J. Appl. Crystallogr. 40:658-674 (2007)). The crystals belong to P212121 space group. The structure was solved by molecular replacement by Phaser (Couture et al., Nat Struct Mol Biol 13:698-703 (2006)) using the previously published WDR5 structure (2H14) (Adams et al., Acta Crystallogr. D Biol. Crystallogr. 58:1948-1954 (2002)). There is one WDR5 molecule in one asymmetric unit. The structure was refined in Phenix (Emsley and Cowtan, Acta Crystallogr. D Biol. Crystallogr. 60:2126-2132 (2004)) with manual model building in Coot (Vagin and Teplyakov, J. Appl. Crystallogr. 30:1022-1025 (2000)). The final model has good stereochemistry with an R-value of 19.0% and an R_{free} of 24.7%.

The WDR5/Cpd. No. 5 complex was formed by diluting the protein to 7 mg/mL with 50 mM Bis-Tris pH 6.5, and 150 mM NaCl, then incubated with Cpd. No. 5 in a 1:1.1 molar ratio for 1 hr at 4° C. Crystals formed at 20° C. in drops containing equal volumes of protein solution and precipitant (0.1 M Na Bis-Tris pH 6.5, 26% PEG 8000 and 0.1 M ammonium sulfate). The crystals were cryoprotected with well solution containing 20% ethylene glycol. Diffraction data were collected at 0.9787 Å wavelength on a Rayonix-MX300 detector at LS-CAT 21-ID-F beamline at the Advanced Photon Source, then processed with HKL2000 (McCoy et al., J. Appl. Crystallogr. 40:658-674 (2007)). WDR5 in complex with Cpd. No. 5 crystallized in C2 space group with 1 molecule of WDR5 per asymmetric unit. The structure was solved to 1.64 Å via molecular replacement (Bricogne et al., BUSTER, 2.10.0, Global Phasing Ltd., Cambridge, United Kingdom (2011)) with WDR5 (PDB ID: 3SMR) as the search model. Iterative rounds of electron density fitting and refinement were completed using Coot (Vagin and Teplyakov, J. Appl. Crystallogr. 30:1022-1025 (2000)) and BUSTER, respectively. The coordinates and geometric restraints for each compound were created from smiles using Grade with the qm+mogul option. The first seven residues are disordered in all the structures.

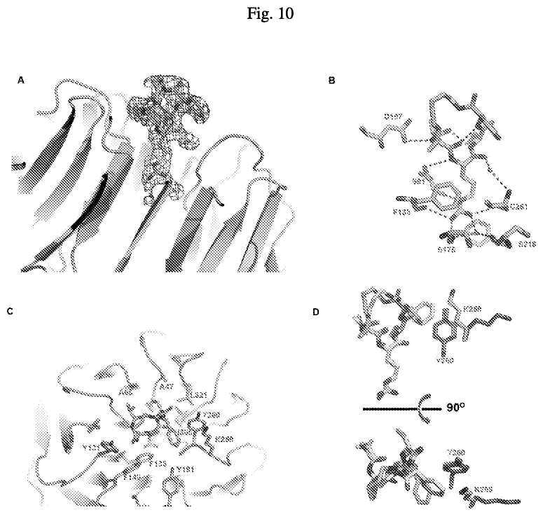

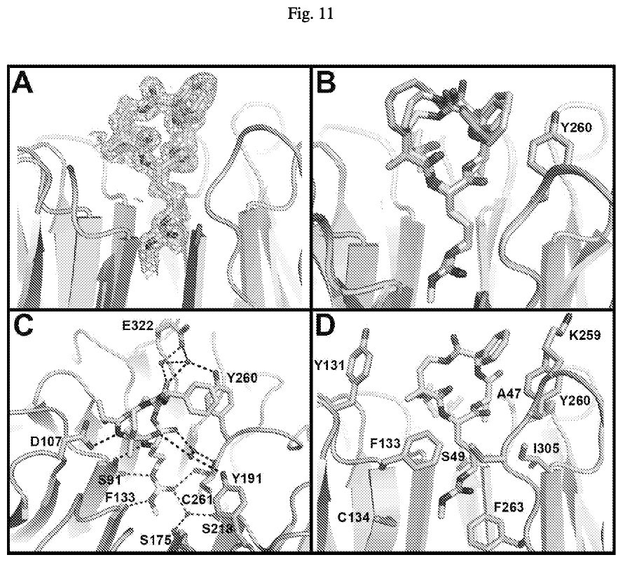

A co-crystal structure of Cpd. No. 64 in complex with WDR5 has been previously reported (Li et al., Eur. J. Med. Chem. 124:480-489 (2016). FIG. 10 shows a co-crystal structure for Cpd. No. 76 at a resolution of 2.8 Å and FIG. 11 shows a co-crystal structure for Cpd. No. 5 in complex with WDR5 at a resolution of 1.64 Å.

WDR5 component in the co-crystal structures of Cpd. No. 76 and Cpd. No. 5, adopt the same β-propeller configuration as the apo-WDR5 structure, with RMSDs (root-main-square deviation) of 0.505 Å and 0.511 Å, respectively. The compounds were unambiguously placed into the central channel of WDR5 through the guidance of difference electron density maps. (FIG. 10A and FIG. 11A). Cpd. No. 76 and Cpd. No. 5 bind to the central channel of WD40 propeller in WDR5 through the conserved interaction network as observed in WDR5/MLL and WDR5/2 complex structures (Li et al., Eur. J. Med. Chem. 124:480-489 (2016)). In agreement with the previous WDR5/Cpd. No. 64 structure, the arginine moiety in Cpd. No. 76 and Cpd. No. 5, which is sandwiched between two phenyl rings from Phe133 and Phe263, engages in a complex array of hydrogen bonding with WDR5. The guanidinium moiety in Cpd. No. 76 and Cpd. No. 5 takes part in direct hydrogen bonds with Cys261, Phe133, and Ser91, and water-mediated hydrogen bonds with Ser218. The guanidinium moiety in Cpd. No. 76 also forms a water-mediated hydrogen bond with Ser175. The N-terminal amide group of Arginine in Cpd. No. 76 and Cpd. No. 5 forms a hydrogen bond with hydroxyl group of Ser91, and C-terminal carbonyl group of Arginine engages in a water-mediated hydrogen bond with amide group of Cys261. Cpd. No. 76 and Cpd. No. 5 adopt a compact bound conformation mediated by 5 direct and 5 or 4 water mediated hydrogen bonds to WDR5 (FIG. 10C).

The methyl group next to the guanidinium moiety in Cpd. No. 5 gained hydrophobic interactions with Cys134 and Phe263 (FIG. 11D). Complementary to these hydrogen bonds, the aliphatic carbon linker and side chains (methyl, isopropyl, ethyl and phenyl groups) of both Cpd. No. 76 and Cpd. No. 5 make extensive hydrophobic packing against the hydrophobic surfaces surrounded by the side chains of Tyr131, Phe133, Tyr191, Tyr260, Leu321, Ile305, Ala47 and Ala65 from WDR5 (FIG. 10C and FIG. 11D). The different linker length between Cpd. No. 64, Cpd. No. 76, and Cpd. No. 5 leads to different orientations of the phenyl group in these compounds (FIG. 10D and FIG. 11B). In Cpd. No. 64 and Cpd. No. 5, the phenyl group stacks nearly parallel to the phenyl group of WDR5 Tyr260. In contrast, the phenyl group in Cpd. No. 76 is oriented nearly perpendicular to the phenyl group of WDR5 Tyr260, comprising an incomplete aromatic cage. Interestingly, the side chain of WDR5 Lys259 is surrounded by an aromatic cage through cation-π stacking interactions (FIG. 10D and FIG. 11D), which is reminiscent of the interaction mode observed in chromodomain recognition of methyl lysine (Nielsen et al., Nature 416:103-107 (2002)). Comparing with Cpd. No. 64 and Cpd. No. 76, the smaller ring size of Cpd. No. 5 forms a more compact structure, which facilitates the formation of several optimal intramolecular hydrogen bonds and reduces its conformational flexibility, allowing this compound to achieve a very high affinity to WDR5 and consequently high potency to inhibit the MLL HMT activity.

### Example 5

### Cell Viability Assay of Leukemia Cells

To assess cell viability, MOLM-13 leukemia cells carrying MLL-AF9 fusion, MV4;11 leukemia cells carrying MLL-AF4 fusion or HL-60 leukemia cells carrying no MLL fusion were seeded 1×10⁴ cell/well in 96-well plates and treated with an inhibitor for 4 days at different concentrations, in culture media containing 0.2% DMSO as the final concentration. Cell viability was determined using the WST-8 cell proliferation assay kit (Dojindo Molecular Technologies) according to manufacturer's instructions. Three independent experiments in triplicates were performed. Data were analyzed using Prism software to determine 50% of cell growth inhibition (IC₅₀) values versus DMSO control. See Table 2 and Table 2A.

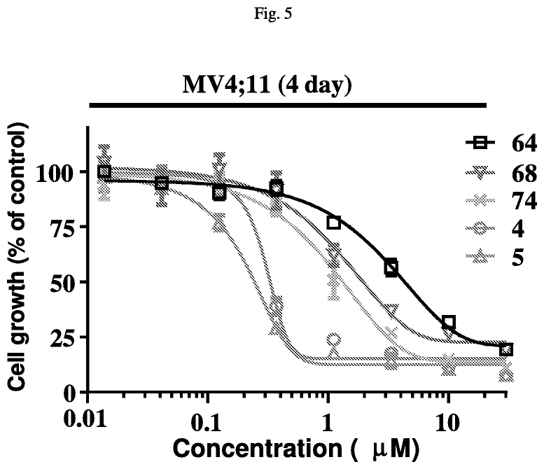

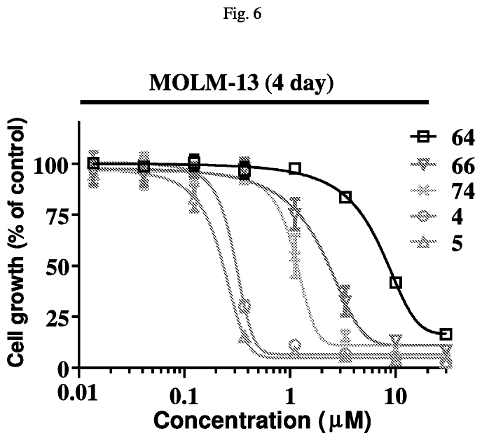

Cpd. No. 5 achieves potent cell growth inhibitory activity in both the MV4;11 (FIG. 5) and MOLM-13 (FIG. 6) cell lines with IC₅₀ values of 0.25 μM and 0.21 μM, respectively. Cpd. No. 5 has weaker activity in the inhibition of cell growth of the HL-60 cell line with an IC₅₀ value of 8.6 μM thus displaying>30-fold selectivity for the MV4;11 and MOLM-13 cell lines harboring MLL translocations over the HL-60 cell line lacking MLL translocation (data not shown).

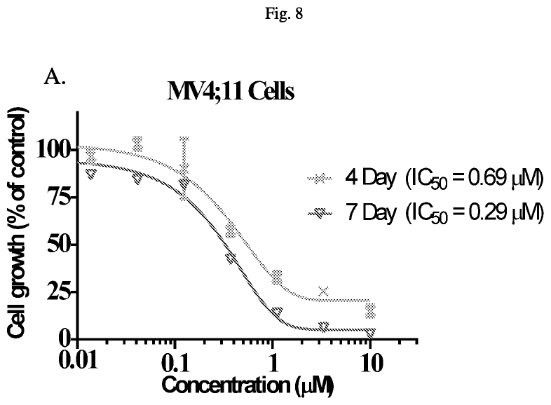

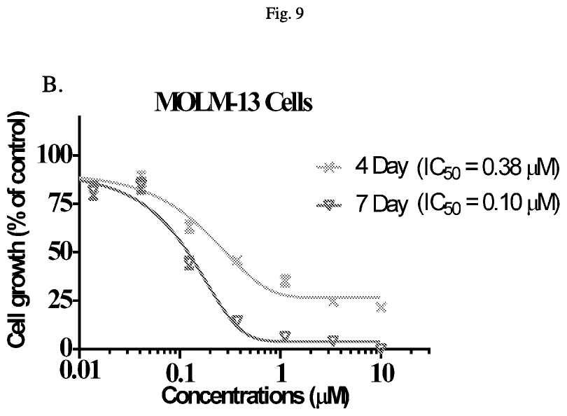

To assess the effect of long-term treatment of compound Cpd. No. 5 on leukemia cells, MOLM-13 and MV4;11 cell lines were plated at a density of 5×10⁴ cell/ml in 24-well plates (2 ml/well), and treated with the relevant concentrations. On day 4, cell viability for each treatment was measured using the WST-8 cell count kit. Then 10% of viable cells from each well were transferred to freshly prepared medium containing corresponding concentrations of compound Cpd. No. 5 and cultured for additional 3 days. On day 7, cell viability for each treatment was determined. See FIG. 8 and FIG. 9. In this experiment, 100,000 cells were seeded in each well in 24-well plates, whereas the cell growth inhibition data obtained for Cpd. No. 64 in Table 2 was obtained by seeding 10,000 cells in 96-well plates. Additionally, Cpd. No. 5 is stable in cell culture media up to 7 days (data not shown).

TABLE 2A

| | MOLM-13 |
|---|---|
| Cpd. No. | IC₅₀ (μM) |
| 6 | >10 |
| 7 | >10 |
| 8 | >10 |
| 9 | >10 |
| 10 | >10 |
| 11 | >10 |
| 12 | >10 |
| 13 | >10 |
| 14 | >10 |
| 15 | >10 |
| 16 | >10 |
| 17 | 0.088 |
| 18 | >10 |
| 19 | >10 |
| 20 | >10 |
| 21 | >10 |
| 22 | >10 |
| 23 | >10 |
| 24 | >10 |
| 25 | >10 |
| 40 | 0.88 |
| 30 | 0.67 |
| 31 | >10 |
| 32 | >10 |
| 33 | 6.5 |
| 34 | >10 |
| 35 | >10 |
| 36 | >10 |
| 26 | 1.02 |
| 43 | >10 |
| 52 | 1.49 |
| 45 | 3.4 |
| 46 | >10 |
| 47 | >10 |
| 78 | >10 |
| 80 | 0.42 |
| 86 | >10 |
| 81 | 1.29 |
| 82 | 0.054 |
| 79 | 0.35 |
| 48 | 1.72 |
| 49 | 1.88 |
| 50 | 1.19 |
| 51 | 0.64 |
| 53 | 0.38 |
| 54 | 1.57 |
| 55 | 3.59 |
| 56 | 1.29 |
| 57 | 6.51 |
| 58 | 2.37 |
| 83 | 2.6 |
| 84 | 0.51 |
| 87 | 0.46 |
| 88 | 6.33 |
| 89 | >10 |
| 90 | >10 |
| 91 | 0.47 |
| 92 | 0.033 |
| 93 | 0.083 |
| 94 | 0.56 |
| 95 | >10 |
| 96 | >10 |
| 97 | >10 |
| 98 | 0.039 |
| 99 | 1.6 |
| 100 | 2.2 |

### Example 6

### Chemical Synthesis

The synthesis of common intermediates for cyclic peptidomimetics is shown in Scheme 1 and Scheme 2. N-terminal of unnatural amino acids 20, 22a and 22b were protected with Fmoc group to give intermediates 21, 23a, and 23b, respectively (Scheme 1).

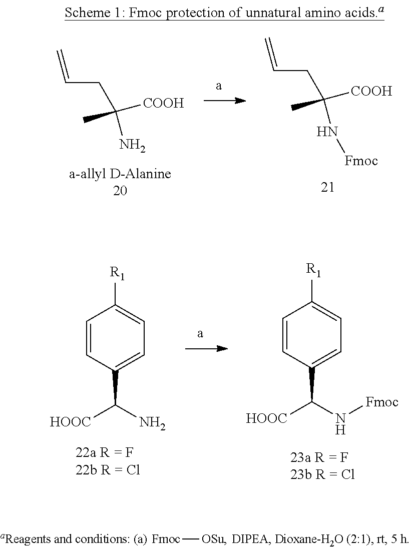

Intermediates 25, 27 and 29 were prepared on the 2-chlorotrityl chloride resin (24) using solid phase peptide synthesis with Fmoc chemistry and cleaved from the resin to yield carboxylic acids 26, 28 and 30, respectively (Scheme 2).

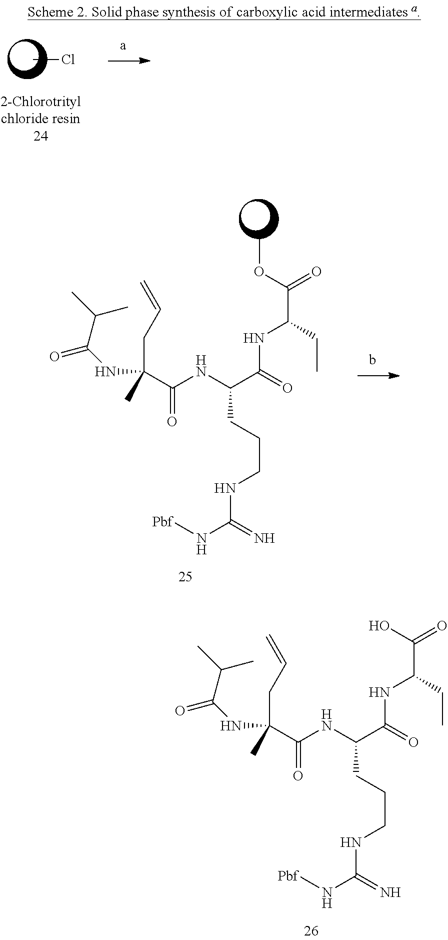

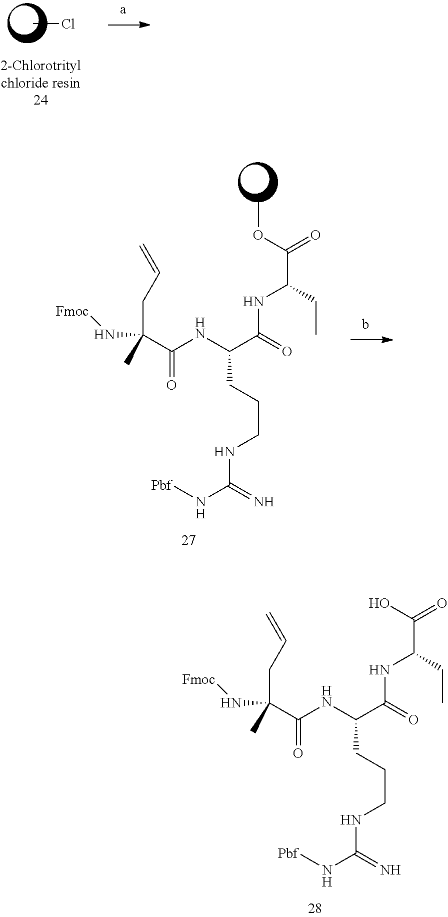

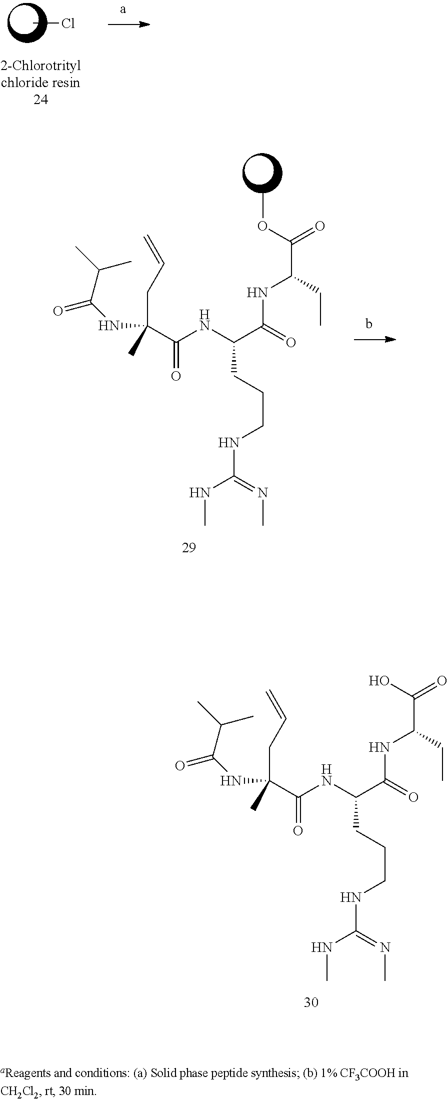

The synthesis of Cpd. Nos. 64-69 and 75-77 is shown in Scheme 3. An alkeneamine (31a-31e) was first attached to an Fmoc-phenylglycine (23a-23c) yielding 32a-32g. Fmoc protecting group from 32a and 32e was then removed with diethylamine treatment, and the remaining amine was coupled to Fmoc-2-Abu-OH yielding 33a and 33e, respectively. A similar method used to make 33a and 33e was applied for the synthesis of 34a, 34e, 34h and 35a, 35e, and 35h. Intermediates 35f-35g were prepared upon Fmoc deprotection of 32f-32g with diethylamine followed by amide coupling with intermediate 28 synthesized in Scheme 2. Fmoc deprotection of 35a, 35e-35h followed by N-terminal capping with isobutiryl chloride afforded 36a, 36e-36h. Fmoc deprotection of 32b-32d followed by amide coupling with intermediate 26 afforded 36b-36d. The same procedure was applied to achieve 36i starting with 32a and carboxylic acid 30. RCM cyclization of 36a-36h followed by catalytic hydrogenation and removal of the Pbf protecting group from arginine side chain yielded the cyclic peptidomimetics Cpd. Nos. 64-68 and 75-77 as trifluoroacetic acid salt. For the synthesis of Cpd. No. 69, 36i was subjected to RCM cyclization, followed by catalytic hydrogenation and treatment with acid to obtain Cpd. No. 69 as trifluoroacetic acid salt.

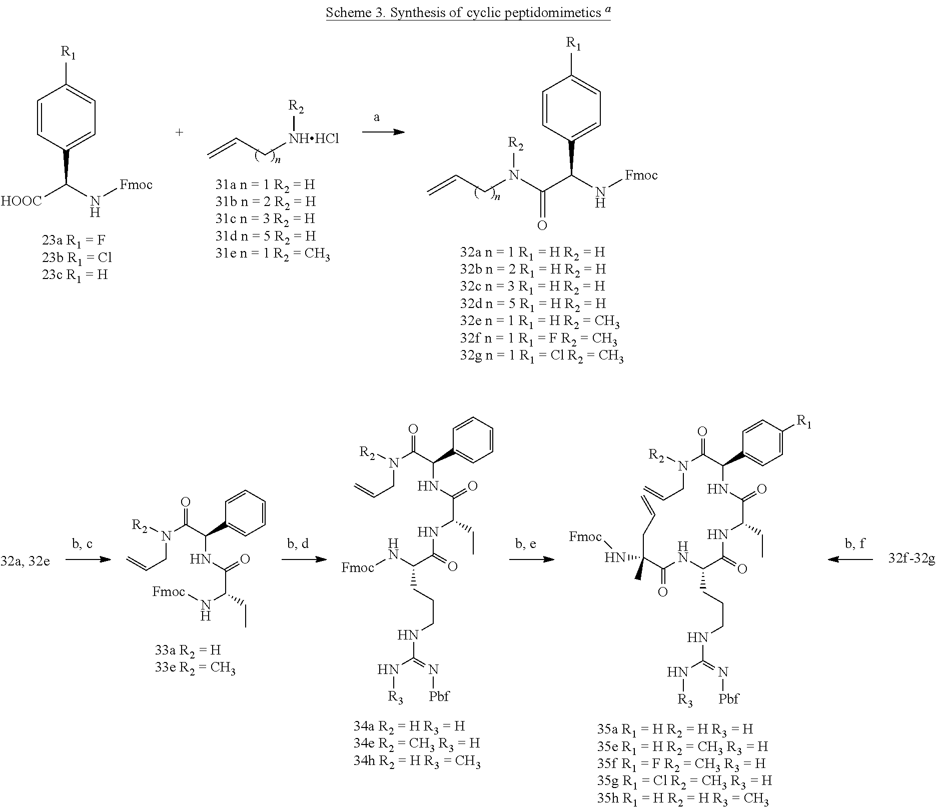

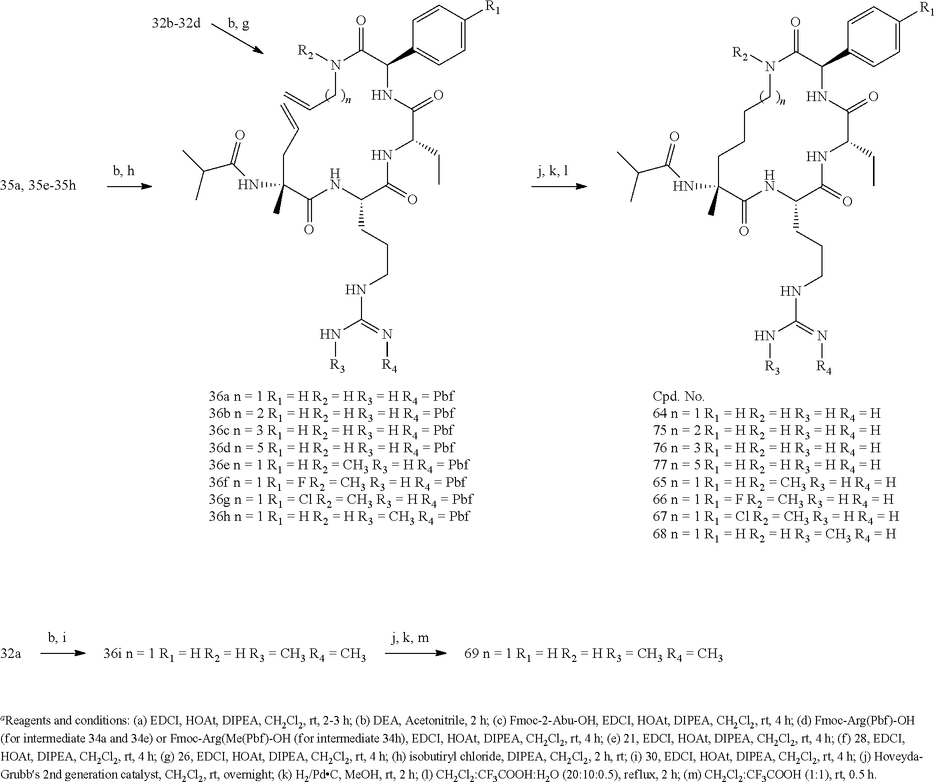

The synthesis of Cpd. Nos. 1, 4, 5, 62, and 71-74 is shown in Scheme 4 and Scheme 5. Intermediates 37a-37e and 41a-41b were prepared on the 2-chlorotrityl chloride resin (24) using solid phase peptide synthesis with Fmoc chemistry. The intermediate peptides were cleaved from the resin followed by Boc or Benzyl protecting group removal with 10% trifluoroacetic acid in dichloromethane or hydrogenation with Pd/C in ethanol, respectively, to yield peptides 38a-38e (Scheme 4) and 42a-42c (Scheme 5). Intramolecular amide coupling of 38a-38e, 42a-42c and followed by removal of Pbf protecting group from arginine side chain yielded Cpd. Nos. 1, 4, 5, 62, and 71-74 as the trifluoroacetic acid salt.

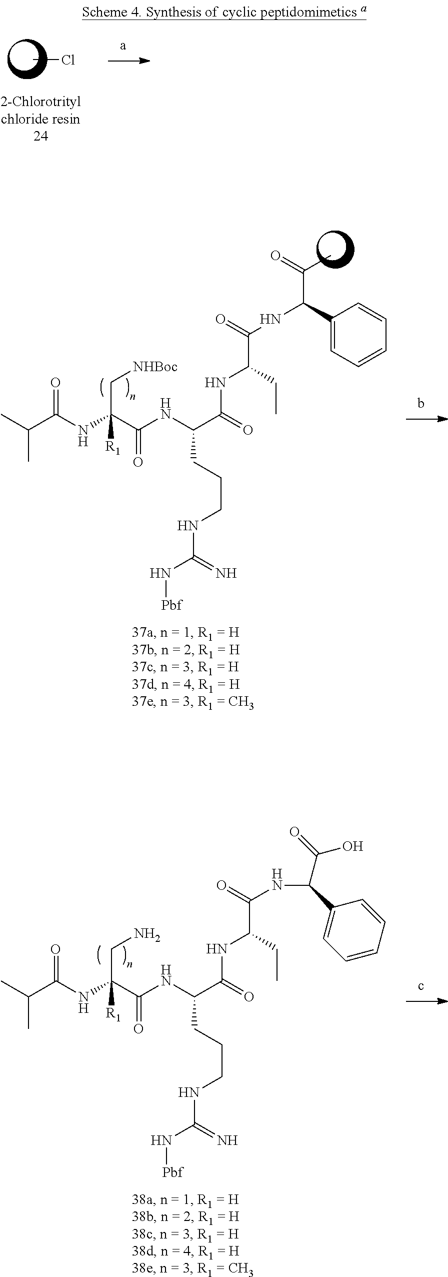

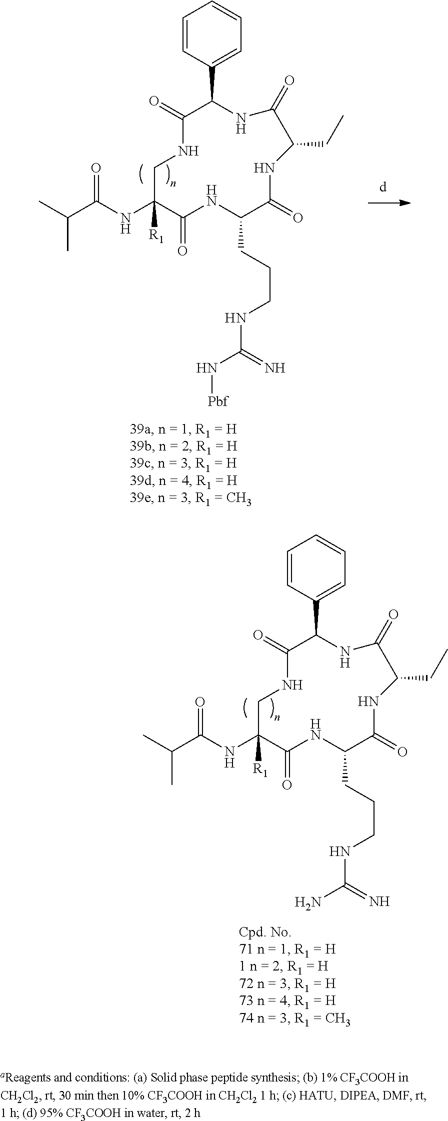

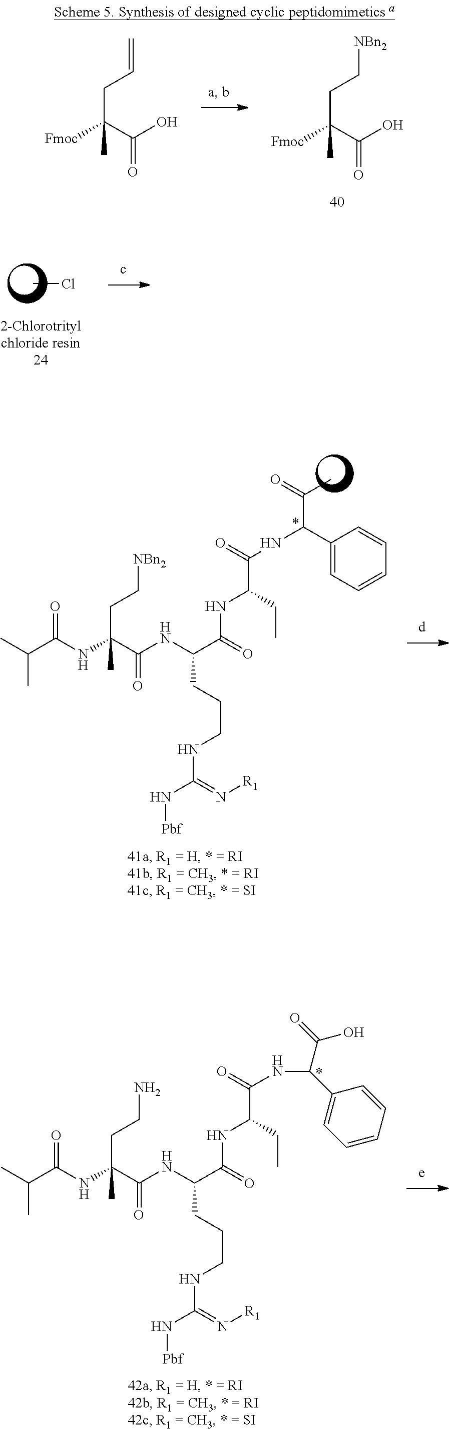

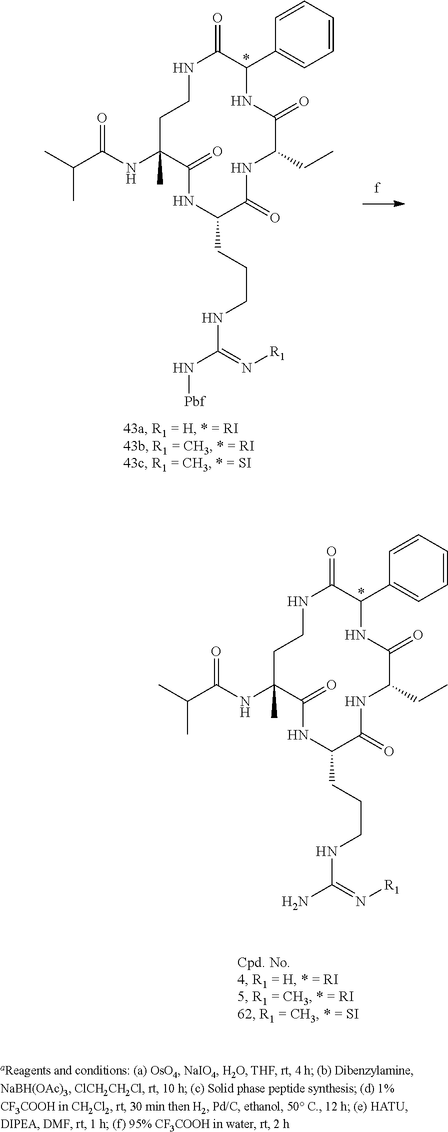

### Synthesis of (R)-2-((((9H-fluoren-9-yl)methoxy)carbonyl)amino)-2-methylpent-4-enoic acid (21)

Intermediate 20 (R-α-allylalanine.H₂O (Nagase)) (750 mg; 5.1 mmol) was mixed with Fmoc-OSu (7.7 mmol 2.6 g) and DIPEA (7.7 mmol, 1.3 mL) in 60 mL 1,4-dioxane:H₂O (2:1). The reaction was stirred at room temperature overnight. 1,4-Dioxane was evaporated, the remaining mixture was neutralized with 1N HCl and extracted to ethyl acetate. The organic layers were collected and dried over anhydrous sodium sulfate, filtered, evaporated and purified over flash chromatography using CH₂Cl₂:MeOH (100:0.5) yielding 1.42 g of (21) as white solid (80% yield). MS (ESI): m/z calc. for [M+H]⁺ 352.15, found 352.40. ¹H NMR (300 MHz, CD3OD) δ: 7.79 (d, J=7.4 Hz, 2H), 7.66 (d, J=7.4 Hz, 2H), 7.39 (t, J=7.4 Hz, 2H), 7.31 (dt, J=1.1 Hz, 7.4 Hz, 2H), 5.81-5.63 (m, 1H), 5.15-5.01 (m, 2H), 4.39-4.27 (m, 2H), 4.21 (t, J=6.6 Hz, 1H), 2.78-2.53 (m, 2H), 1.44 (s, 3H).

A procedure similar to applied for intermediate 21 was used for 23a and 23b starting from 22a and 22b, respectively. Quantitative yields were obtained for both intermediates.

### Synthesis of (9H-fluoren-9-yl)methyl (R)-2-((2-(allylamino)-2-oxo-1-phenylethyl)amino)-2-oxoacetate (32a)

Fmoc-R-phenylglycine (23c) (2.7 mmol; 1 g) was mixed with allylamine hydrochloride (31a) (4.1 mmol, 0.38 g), EDCl (4.1 mmol, 0.79 g), HOAt (4.1 mmol, 0.55 g), 3 equiv DIPEA (8.1 mmol, 1.4 mL) in 100 mL CH₂Cl₂ and stirred at room temperature for 3h. The reaction mixture was quenched with H₂O and extracted to CH₂Cl₂. The organic layer together with a white precipitate was collected and concentrated in vacuo. The remaining white solid was washed from CH₂Cl₂ yielding 0.96 g intermediate (32a) as white powder (86% yield). HRMS (ESI): m/z calc. for C₂₆H₂₅N₂O₃ [M+H]⁺ 413.1860, found 413.1862. ¹H NMR (300 MHz, CD₃CN) δ: 7.85 (d, J=7.5 Hz, 2H), 7.68 (d, J=7.0 Hz, 2H), 7.47-7.29 (m, 9H), 6.83 (brs, 1H), 6.47 (brs, 1H), 5.85-5.71 (m, 1H), 5.16 (d, J=6.6 Hz, 1H), 5.07-4.96 (m, 2H), 4.35 (d, J=6.8 Hz, 2H), 4.24 (t, J=6.9 Hz, 1H), 3.81-3.74 (m, 2H). ¹³C NMR (75 MHz, CD₃CN): δ 170.75, 145.28, 145.18, 142.26, 135.61, 129.81, 129.24, 128.81, 128.42, 128.24, 126.30, 121.09, 115.87, 67.53, 59.96, 48.18, 42.31.

### Synthesis of (S)-2-((S)-2-((R)-2-isobutyramdo-2-methylpent-4-enamido)-5-(3-((2,2,4,6,7-pentamethyl-2,3-dihydrobenzofuran-5-yl)sulfonyl)guanidino)pentanamido) butanoic acid (26)

Fmoc-2-Abu-OH (8.1 mmol, 2.6 g) was loaded on the 2.7 mmol 2-chlorotrityl chloride (24) resin (ChemPep) (1.2 mmol/g) overnight in CH₂Cl₂ and in the presence of DIPEA (8.1 mmol, 1.4 mL). Then, the resin was washed with DMF, MeOH, CH₂Cl₂, respectively, mixed with DIPEA (0.29 mmol, 0.5 mL) in MeOH:CH₂Cl₂ (1:5) and was shaken for 30 min to endcap unreacted 2-chlorotrityl group on the resin. Next, classical chain elongation was carried out with Fmoc chemistry. The carboxylic acid intermediate (26) was cleaved from the resin by treatment of 25 with 4 ml of 1% trifluoroacetic acid in CH₂Cl₂ (3×10 min). The filtrate was evaporated and the remaining crude product was purified with preparative HPLC using the C18 reverse phase column (Waters, Sunfire™ Prep Cis OBD™, 5 μm, 50×100 mm), yielding 0.9 g white powder (70% yield). MS (ESI): m/z calculated for [M+H]⁺ 693.37, found 693.42. ¹H NMR (300 MHz, CD₃OD) δ: 5.79-5.63 (m, 1H), 5.12-5.03 (m, 2H), 4.45-4.36 (m, 1H), 4.24 (dd, J=5.2, 8.4 Hz, 1H), 3.24-3.15 (m, 2H), 3.00 (s, 2H), 2.79-2.67 (m, 1H), 2.60-2.43 (m, 8H), 2.08 (s, 3H), 1.99-1.51 (m, 6H), 1.45 (s, 6H), 1.40 (s, 3H), 1.07 (d, J=6.8 Hz, 6H), 0.97 (t, J=7.4 Hz, 3H). ¹³C NMR (75 MHz, CD₃OD) δ: 179.89, 176.19, 175.20, 174.09, 133.74, 126.49, 119.96, 118.86, 88.10, 60.30, 60.21, 55.49, 54.00, 44.04, 41.91, 36.07, 30.31, 28.89, 25.75, 23.06, 20.17, 19.91, 19.77, 18.48, 12.71, 11.03.

### Synthesis of (5R,8S,11S)-5-allyl-11-ethyl-1-(9H-fluoren-9-yl)-5-methyl-3,6,9-trioxo-8-(3-(3-((2,2,4,6,7-pentamethyl-2,3-dihydrobenzofuran-5-yl)sulfonyl)guanidino) propyl)-2-oxa-4,7,10-triazadodecan-12-oic acid (28)

Intermediate 28 was synthesized using the procedure applied for 26. (70% yield). MS (ESI): m/z calc. for [M+H]⁺ 845.52, found 845.39. ¹H NMR (300 MHz, CD₃OD) δ: 7.79 (d, J=7.4 Hz, 2H), 7.64 (d, J=7.4 Hz, 2H), 7.38 (t, J=7.4 Hz, 2H), 7.30 (ddt, J=1.2, 2.9, 7.4 Hz, 2H), 5.76-5.59 (m, 1H), 5.11-4.98 (m, 2H), 4.49-4.28 (m, 3H), 4.28-4.13 (m, 2H), 3.15 (t, J=6.6 Hz, 2H), 2.96 (s, 2H), 2.70-2.43 (m, 8H), 2.06 (s, 3H), 1.97-1.47 (m, 6H), 1.42 (s, 6H), 1.36 (s, 3H), 0.95 (t, J=7.4 Hz, 3H). ¹³C NMR (75 MHz, CD₃OD) δ: 176.58, 175.20, 174.16, 157.67, 145.42, 145.39, 142.80, 133.80, 128.98, 128.36, 128.34, 126.36, 126.30, 121.13, 120.00, 67.87, 60.57, 55.51, 44.03, 42.30, 30.06, 28.85, 25.87, 23.35, 19.77, 18.46, 12.69, 11.00.

### Synthesis of (S)-2-((S)-5-((E)-2,3-dimethylguanidino)-2-((R)-2-isobutyramido-2-methylpent-4-enamido)pentanamido)butanoic acid (30)

Intermediate 30 was synthesized using the procedure applied for 26. (38% yield). MS (ESI): m/z calc. for [M+H]⁺ 469.31, found 469.50. ¹H NMR (300 MHz, CD₃OD) δ: 5.81-5.66 (m, 1H), 5.15-5.05 (m, 2H), 4.47-4.41 (m, 1H), 4.26 (dd, J=5.1, 8.4 Hz, 1H), 3.27-3.13 (m, 2H), 2.85 (s, 6H), 2.71 (dd, J=7.6, 13.8 Hz, 1H), 2.58-2.47 (m, 2H), 2.00-1.58 (m, 6H), 1.42 (s, 3H), 1.13-1.06 (m, 6H), 0.99 (t, J=7.4 Hz, 3H). ¹³C NMR (75 MHz, CD₃OD) δ: 180.00, 176.21, 175.23, 174.05, 157.46, 133.70, 119.95, 60.31, 55.50, 53.78, 42.40, 42.13, 36.08, 30.55, 28.49, 25.99, 25.83, 22.86, 20.13, 19.89, 10.97.

### Synthesis of (R)-(9H-fluoren-9-yl)methyl 2-((2-(but-3-en-1-ylamino)-2-oxo-1-phenylethyl)amino)-2-oxoacetate (32b)

Fmoc-R-Phenylglycine (23c) (1 mmol; 0.37 g) was mixed with 3-butenyl amine hydrochloride (31b, 1.5 mmol, 0.16 g), EDCl (1.5 mmol, 0.29 g), HOAt (1.5 mmol, 0.21 g), DIPEA (3 mmol, 0.52 mL) in 40 mL CH₂Cl₂ and stirred at room temperature for 4h. The reaction mixture was quenched with H₂O and extracted to CH₂Cl₂. The organic layers were collected and dried over anhydrous Na₂SO₄, filtered, evaporated and purified over flash chromatography using CH₂Cl₂:EtOAc (1:1) to afford 0.41 g of 32b as white solid (96% yield). MS (ESI): m/z calc. for [M+H]⁺ 427.20, found 427.25. ¹H NMR (300 MHz, CDCl₃) δ: 7.75 (d, J=7.5 Hz, 2H), 7.57 (brs, 2H), 7.45-7.23 (m, 9H), 6.32-6.21 (m, 1H), 5.77 (brs, 1H), 5.70-5.53 (m, 1H), 5.18 (d, J=5.3 Hz, 1H), 5.01-4.83 (m, 2H), 4.35 (d, J=6.5 Hz, 2H), 4.19 (t, J=6.9 Hz, 1H), 3.41-3.18 (m, 2H), 2.24-2.09 (m, 2H). ¹³C NMR (75 MHz, CDCl₃) δ: 169.76, 155.88, 144.02, 143.96, 141.45, 134.77, 129.30, 128.71, 127.88, 127.41, 127.27, 125.28, 120.16, 117.76, 67.32, 59.09, 47.30, 38.87, 33.71.

### Synthesis of (R)-(9H-fluoren-9-yl)methyl 2-oxo-2-((2-oxo-2-(pent-4-en-1-ylamino)-1-phenylethyl)amino)acetate (32c)

Intermediate 32c was prepared according to the procedure used for 32b starting from 23c and 31c. The product was purified over flash chromatography using hexanes:EtOAc (3:1) to afford 32c as white solid (84% yield). MS (ESI): m/z calc. for [M+H]⁺ 441.22, found 441.50. ¹H NMR (300 MHz, CD₃OD) δ: 7.79 (d, J=7.5 Hz, 2H), 7.65 (d, J=7.2 Hz, 2H), 7.44-7.24 (m, 9H), 5.83-5.67 (m, 1H), 5.19 (s, 1H), 4.99-4.88 (m, 2H), 4.37 (d, J=6.6 Hz, 2H), 4.22 (t, J=6.6 Hz, 1H), 3.27-3.10 (m, 2H), 2.03-1.92 (m, 2H), 1.60-1.48 (m, 2H). ¹³C NMR (75 MHz, CD₃OD) δ: 172.95, 145.38, 142.78, 139.21, 129.95, 129.46, 128.99, 128.67, 128.38, 126.41, 121.12, 115.67, 68.31, 60.64, 40.23, 32.18, 29.77.

### Synthesis of (R)-(9H-fluoren-9-yl)methyl 2-((2-(hept-6-en-1-ylamino)-2-oxo-1-phenylethyl)amino)-2-oxoacetate (32d)

Intermediate 32d was prepared according to the procedure used for 32b starting from 23c and 31d. The product was purified over flash chromatography using CH₂Cl₂:EtOAc (3:1) and the product was further washed with n-hexanes to afford 32d as white solid (80% yield). MS (ESI): m/z calc. for [M+H]⁺ 469.25, found 469.00. ¹H NMR (300 MHz, CD₃OD) δ: 7.73 (d, J=7.5 Hz, 2H), 7.58 (d, J=7.0 Hz, 2H), 7.41-7.21 (m, 9H), 5.79-5.63 (m, 1H), 5.17 (s, 1H), 4.97-4.83 (m, 2H), 4.35 (d, J=6.7 Hz, 2H), 4.18 (t, J=6.7 Hz, 1H), 3.25-3.06 (m, 2H), 2.01-1.89 (m, 2H), 1.51-1.13 (m, 6H). ¹³C NMR (75 MHz, CD₃OD) δ: 171.72, 157.16, 144.60, 144.51, 142.06, 139.40, 129.49, 128.96, 128.45, 127.83, 127.77, 125.78, 120.63, 114.88, 67.82, 59.48, 47.87, 40.29, 34.32, 29.66, 29.20, 26.91.

### Synthesis of (R)-(9H-fluoren-9-yl)methyl (2-(allyl(methyl)amino)-2-oxo-1-phenylethyl)carbamate (32e)

Intermediate 32e was prepared according to the procedure used for 32b starting from 23c and 31e. The product was purified over flash chromatography using hexanes:EtOAc (1:1). White solid (84% yield). MS (ESI): m/z calc. for [M+H]⁺ 427.20, found 427.08. ¹H NMR (300 MHz, CD₃OD, rotamers) δ: 7.85-7.70 (m, 2H), 7.62-7.47 (m, 2H), 7.46-7.16 (m, 9H), 5.76-5.41 (m, 2H), 5.13-4.88 (m, 2H), 4.36-3.65 (m, 5H), 2.88 (s, 1.3H), 2.84 (s, 1.5H), 2.76 (s, 0.2H). ¹³C NMR (75 MHz, CD₃OD, rotamers) δ: 172.32, 172.10, 158.01, 145.46, 145.29, 142.68, 138.58, 138.07, 133.55, 133.46, 130.24, 129.73, 129.41, 129.31, 128.93, 128.33, 126.46, 126.42, 121.07, 118.31, 117.84, 68.31, 57.61, 57.52, 52.99, 51.75, 48.50, 35.35, 34.36.

### Synthesis of (R)-(9H-fluoren-9-yl)methyl(2-(allyl(methyl)amino)-1-(4-fluorophenyl)-2-oxoethyl)carbamate (32f)

Intermediate 32f was prepared according to the procedure used for 32b starting from 23a and 31e. The product was purified over flash chromatography using hexane:ethylacetate (3:1). White solid (75% yield). MS (ESI): m/z calc. for [M+H]⁺ 445.19, found 444.96. ¹H NMR (300 MHz, CD₃OD, rotamers) δ: 7.88-7.72 (m, 2H), 7.65-7.50 (m, 2H), 7.47-7.19 (m, 6H), 7.14-6.78 (m, 2H), 5.80-5.51 (m, 2H), 5.17-5.03 (m, 2H), 4.42-4.19 (m, 3H), 4.11-3.88 (m, 2H), 2.90 (s, 1.2H), 2.87 (s, 1.5H), 2.77 (s, 0.3H). ¹³C NMR (75 MHz, CD₃OD, rotamers) δ: 172.25, 172.00, 165.91, 162.64, 158.03, 157.98, 145.48, 145.31, 142.74, 134.73, 134.69, 134.21, 134.16, 133.54, 133.49, 131.54, 131.44, 131.35, 128.96, 128.34, 128.32, 126.43, 126.41, 121.09, 118.25, 117.90, 117.06, 116.77, 68.29, 56.80, 56.68, 53.03, 51.80, 48.56, 35.37, 34.44.

### Synthesis of (R)-(9H-fluoren-9-yl)methyl(2-(allyl(methyl)amino)-1-(4-chlorophenyl)-2-oxoethyl)carbamate (32g)

Intermediate 32g was prepared according to the procedure used for 32b starting from 23b and 31e. The product was purified over flash chromatography using hexane:ethylacetate (3:1). White solid (75% yield). MS (ESI): m/z calc. for [M+H]⁺ 461.16, found 461.19. ¹H NMR (300 MHz, CD₃OD, rotamers) δ: 7.88-7.72 (m, 2H), 7.65-7.50 (m, 2H), 7.47-7.20 (m, 6H), 7.14-7.04 (m, 2H), 5.78-5.47 (m, 2H), 5.15-5.01 (m, 2H), 4.38-4.25 (m, 2H), 4.23-4.13 (m, 1H), 4.08-3.85 (m, 2H), 2.90 (s, 1.2H), 2.87 (s, 1.5H),2.77 (s, 0.3H). ¹³C NMR (75 MHz, CD₃OD, rotamers) δ: 171.86, 171.59, 157.87, 145.39, 145.21, 142.65, 137.45, 136.95, 135.51, 133.45, 133.40, 131.00, 130.93, 130.24, 128.92, 128.30, 126.37, 121.07, 118.28, 117.95, 68.23, 56.74, 56.59, 52.98, 51.74, 48.48, 35.36, 34.44.

### Synthesis of (9H-fluoren-9-yl)methyl ((S)-1-(((R)-2-(allylamino)-2-oxo-1-phenylethyl)amino)-1-oxobutan-2-yl)carbamate (33a)

Intermediate 32a (1.65 mmol; 0.68 g) was treated with diethylamine (33 mmol, 3.5 mL) in 100 mL CH₃CN at 40° C. for 4h followed by removal of the solvent and diethylamine in vacuo. The resulting crude product was further dried under vacuum then taken into CH₂Cl₂ and mixed with Fmoc-2-Abu-OH (2.5 mmol, 0.81 g), EDCl (2.5 mmol, 0.48 g), HOAt (2.5 mmol, 0.34 g) and DIPEA (2.5 mmol, 0.44 mL) at room temperature for 4h. The reaction mixture was quenched with H₂O and extracted to CH₂Cl₂. The organic layer together with a white precipitate was collected and concentrated in vacuo. The remaining white solid was washed from CH₂Cl₂ yielding 0.57 g intermediate 33a as white powder (70% yield). HRMS (ESI): m/z calc. for C₃₀H₃₂N₃O₄ [M+H]⁺ 498.2387, found 498.2392. ¹H NMR (300 MHz, CD₃SO) δ: 8.56 (d, J=8.0 Hz, 1H), 8.44 (t, J=5.5 Hz, 1H), 7.89 (d, J=7.5 Hz, 2H), 7.76-7.70 (m, 2H), 7.55 (d, J=8.1 Hz, 1H), 7.45-7.38 (m, 4H), 7.36-7.22 (m, 4H), 5.80-5.67 (m, 1H), 5.48 (d, J=8.0 Hz, 1H), 5.05-4.93 (m, 2H), 4.30-4.03 (m, 4H), 3.73-3.64 (m, 2H), 1.69-1.43 (m, 2H), 0.81 (t, J=7.3 Hz, 3H). ¹³C NMR (75 MHz, CD₃SO) δ: 171.52, 169.41, 156.07, 143.86, 143.75, 140.68, 138.99, 134.70, 128.22, 127.61, 127.45, 127.07, 126.88, 125.25, 120.07, 115.14, 65.67, 56.02, 55.91, 46.65, 40.81, 25.17, 10.35.

Method A: The Fmoc protected intermediate was treated with diethylamine (20 equiv.) in CH₃CN for 2h at room temperature followed by removal of the solvent and diethylamine in vacuo. The resulting crude product was further dried under vacuum, then taken into CH₂Cl₂ and mixed with the corresponding Fmoc-amino acid or peptide carboxylic acid (1.5 equiv.), EDCl (1.5 equiv.), HOAt (1.5 equiv.) and diisopropylethylamine (1.5 equiv.). The reaction mixture was stirred at room temperature for 2-3 h, quenched with H₂O and extracted to CH₂Cl₂. The organic layers were collected and dried over anhydrous Na₂SO₄, filtered, evaporated and purified over flash chromatography.

### Synthesis of (9H-fluoren-9-yl)methyl ((S)-1-(((R)-2-(allyl(methyl)amino)-2-oxo-1-phenylethyl)amino)-1-oxobutan-2-yl)carbamate (33e)

Intermediate 33e was prepared according to Method A starting from 32e (0.7 g; 1.64 mmol). Fmoc-2-Abu-OH used as the amino acid. The compound was purified over flash chromatography using hexanes:EtOAc (1:1) affording 0.68 g intermediate 33e as white solid (81% yield). MS (ESI): m/z calc. for [M+H]⁺ 512.25, found 512.58. ¹H NMR (300 MHz, CD₃OD, 2-rotamers) δ: 7.78 (d, J=7.5 Hz, 2H), 7.64 (d, J=7.3 Hz, 2H), 7.42-7.24 (m, 9H), 5.85 (s, 0.55H), 5.83 (s, 0.45H), 5.77-5.42 (m, 1H), 5.14-4.97 (m, 2H), 4.39-4.24 (m, 2H), 4.22-4.00 (m, 3H), 3.96-3.82 (m, 1H), 2.89 (s, 3H), 1.84-1.51 (m, 2H), 0.86 (t, J=7.4 Hz, 3H). ¹³C NMR (75 MHz, CD₃OD, 2-rotamers) δ: 174.01, 173.94, 171.69, 171.46, 158.59, 145.56, 145.35, 142.77, 138.50, 138.01, 133.54, 133.49, 130.25, 129.77, 129.37, 129.29, 128.96, 128.39, 128.35, 126.43, 126.36, 121.11, 118.37, 117.89, 68.21, 58.03, 55.84, 55.71, 53.07, 51.74, 35.37, 34.31, 26.73, 10.76.

### Synthesis of (9H-fluoren-9-yl)methyl ((6S,9S,12R)-9-ethyl-1-imino-7,10,13-trioxo-1-(2,2,4,6,7-pentamethyl-2,3-dihydrobenzofuran-5-sulfonamido)-12-phenyl-2,8,11,14-tetraazaheptadec-16-en-6-yl)carbamate (34a)

A method applied for 33a was used to make 34a starting from 33a. White solid (83% yield). HRMS (ESI): m/z calc. for C₄₉H₆₀N₇O₈S [M+H]⁺ 906.4219, found 906.4222. ¹H NMR (300 MHz, CD₃SO) δ: 8.59 (d, J=8.1 Hz, 1H), 8.45 (t, J=5.7 Hz, 1H), 7.96 (d, J=7.6 Hz, 1H), 7.89 (d, J=7.4 Hz, 2H), 7.75-7.67 (m, 2H), 7.53 (d, J=8.2 Hz, 1H), 7.44-7.37 (m, 4H), 7.35-7.22 (m, 5H), 5.80-5.66 (m, 1H), 5.48 (d, J=8.1 Hz, 1H), 5.04-4.94 (m, 2H), 4.39-4.17 (m, 4H), 4.08-3.96 (m, 1H), 3.72-3.64 (m, 2H), 3.07-2.98 (m, 2H), 2.93 (s, 2H), 2.49 (s, 3H), 2.43 (s, 3H), 1.99 (s, 3H), 1.72-1.35 (m, 12H), 0.77 (t, J=7.3 Hz, 3H). ¹³C NMR (75 MHz, CD₃SO): δ 171.77, 170.96, 169.40, 157.44, 156.05, 155.91, 143.85, 143.72, 140.69, 138.93, 137.26, 134.70, 134.20, 131.43, 128.22, 127.61, 127.47, 127.06, 126.93, 125.26, 124.30, 120.06, 116.25, 115.13, 86.25, 65.62, 56.03, 53.68, 46.67, 42.45, 40.84, 28.25, 25.33, 18.92, 17.58, 12.23, 10.02.

### Synthesis of (9H-fluoren-9-yl)methyl ((6S,9S,12R)-9-ethyl-1-imino-14-methyl-7,10,13-trioxo-1-(2,2,4,6,7-pentamethyl-2,3-dihydrobenzofuran-5-sulfonamido)-12-phenyl-2,8,11,14-tetraazaheptadec-16-en-6-yl)carbamate (34e)

Intermediate 34e was prepared according to Method A starting from 33e (0.66 g 1.29 mmol). Fmoc-Arg(Pbf)-OH used as the amino acid. The compound was purified over flash chromatography using hexanes:EtOAc (1:1) affording 1.0 g (1.09 mmol) intermediate 34e as white solid (84% yield). MS (ESI): m/z calc. for [M+H]⁺ 920.44, found 920.25. ¹H NMR (300 MHz, CD₃OD, 2-rotamers) δ: 7.79 (d, J=7.5 Hz, 2H), 7.66 (t, J=7.2 Hz, 2H), 7.42-7.26 (m, 9H), 5.81 (s, 0.5H), 5.79 (s, 0.5H), 5.74-5.58 (m, 1H), 5.11-4.97 (m, 2H), 4.40-4.29 (m, 3H), 4.21 (t, J=6.5 Hz, 1H), 4.11-3.98 (m, 2H), 3.91-3.80 (m, 1H), 3.19-3.10 (m, 2H), 2.96 (s, 2H), 2.86 (s, 3H), 2.58 (s, 3H), 2.51 (s, 3H), 2.06 (s, 3H), 1.87-1.46 (m, 6H), 1.42 (s, 6H), 0.85 (t, J=7.1 Hz, 3H). 13C NMR (75 MHz, CD₃OD, rotamers) δ:174.86, 173.27, 171.71, 171.54, 160.07, 158.70, 158.33, 145.65, 145.33, 142.80, 139.62, 137.70, 134.63, 133.73, 133.55, 133.46, 130.28, 129.83, 129.52, 129.42, 129.00, 128.38, 126.44, 126.23, 121.13, 118.65, 118.42, 117.82, 87.85, 68.21, 56.42, 56.07, 56.00, 53.08, 51.78, 44.17, 41.77, 35.41, 34.37, 30.63, 28.89, 26.94, 26.41, 19.79, 18.60, 12.71, 10.76.

### Synthesis of (9H-fluoren-9-yl)methyl ((8S,11S,14R,E)-11-ethyl-9,12,15-trioxo-3-(2,2,4,6,7-pentamethyl-2,3-dihydrobenzofuran-5-sulfonamido)-14-phenyl-2,4,10,13,16-pentaazanonadeca-2,18-dien-8-yl)carbamate (34h)

A method similar that applied for 33a was used for the synthesis of 34h starting from 33a (0.5 g, 1 mmol). Fmoc-Arg(Me)(Pbf)-OH was used as the amino acid. 0.36 g intermediate 34h was obtained as white solid (65% yield) and used for the next step without further purification. MS (ESI): m/z calc. for [M+H]⁺ 920.44, found 920.25.

### Synthesis of (9H-fluoren-9-yl)methyl ((6R,9S,12S,15R)-9-ethyl-15-methyl-5,8,11,14-tetraoxo-12-(3-(3-((2,2,4,6,7-pentamethyl-2,3-dihydrobenzofuran-5-yl)sulfonyl)guanidino)propyl)-6-phenyl-4,7,10,13-tetraazaoctadeca-1,17-dien-15-yl)carbamate (35a)

Intermediate 34 (1.18 g; 1.3 mmol) was treated with diethylamine (26 mmol, 2.7 mL) in 50 mL CH₃CN at 40° C. for 2h followed by removal of the solvent and diethylamine in vacuo. The resulting crude product was taken into CH₂Cl₂ and mixed with 21 (2 mmol, 0.7 g), EDCl (2 mmol, 0.38 g), HOAt (2 mmol, 0.27 g) and DIPEA (2 mmol, 0.35 mL) at room temperature for 4h. The reaction mixture was quenched with H₂O and extracted to CH₂Cl₂. The organic layers were collected and dried over anh. sodium sulfate, filtered, evaporated and purified over flash chromatography using EtAc:MeOH (50:0.7) yielding 0.8 g intermediate 35a as white solid (60% yield). HRMS (ESI): m/z calc. for C₅₅H₆₉N₈O₉S [M+H]⁺ 1017.4903, found 1017.4899. ¹H NMR (300 MHz, MeOD) δ: 7.79 (d, J=7.4 Hz, 2H), 7.65 (d, J=7.4 Hz, 2H), 7.43-7.25 (m, 9H), 5.77-5.60 (m, 2H), 5.40 (s, 1H), 5.11-4.92 (m, 4H), 4.52-4.43 (m, 1H), 4.36-4.15 (m, 4H), 3.84-3.64 (m, 2H), 3.17-3.09 (m, 2H), 2.95 (s, 2H), 2.67-2.39 (m, 8H), 2.05 (s, 3H), 1.96-1.45 (m, 6H), 1.41 (s, 6H), 1.34 (s, 3H), 0.89 (t, J=7.2 Hz, 3H). ¹³C NMR (75 MHz, CD₃OD): δ 171.77, 170.96, 169.40, 157.44, 156.05, 155.91, 143.85, 143.72, 140.69, 138.93, 137.26, 134.70, 134.20, 131.43, 128.22, 127.61, 127.47, 127.06, 126.93, 125.26, 124.30, 120.06, 116.25, 115.13, 86.25, 65.62, 56.03, 53.68, 46.67, 42.45, 40.84, 28.25, 25.33, 18.92, 17.58, 12.23, 10.02.

### Synthesis of (9H-fluoren-9-yl)methyl ((6R,9S,12S,15R)-9-ethyl-4,15-dimethyl-5,8,11,14-tetraoxo-12-(3-(3-((2,2,4,6,7-pentamethyl-2,3-dihydrobenzofuran-5-yl)sulfonyl)guanidino)propyl)-6-phenyl-4,7,10,13-tetraazaoctadeca-1,17-dien-15-yl)carbamate (35e)

Intermediate 35e was prepared according to Method A starting from 34e (0.9 g; 1.0 mmol). Intermediate 21 was used as the amino acid. The compound was purified over flash chromatography using hexanes:EtOAc (1:1) affording 0.83 g (0.8 mmol) intermediate 35e as white solid (80% yield). MS (ESI): m/z calc. for [M+H]⁺ 1031.51, found 1031.75. ¹H NMR (300 MHz, CD₃OD, rotamers) δ: 7.79 (d, J=7.4 Hz, 2H), 7.68-7.62 (m, 2H), 7.44-7.24 (m, 9H), 5.78-5.76 (m, 1H), 5.74-5.43 (m, 2H), 5.12-4.96 (m, 4H), 4.59-4.48 (m, 1H), 4.35-4.15 (m, 4H), 4.07-3.77 (m, 2H), 3.17-3.08 (m, 2H), 2.95 (s, 2H), 2.87 (s, 0.2H), 2.85 (s, 1.6H), 2.83 (s, 1.2H),2.64-2.36 (m, 8H), 2.05 (s, 3H), 1.94-1.45 (m, 6H), 1.42 (s, 6H), 1.33 (s, 3H), 0.87 (t, J=7.2 Hz, 3H). ¹³C NMR (75 MHz, CD₃OD) δ:176.90, 174.37, 173.49, 171.57, 171.37, 160.05, 158.29, 157.88, 145.51, 145.44, 142.87, 142.85, 139.60, 137.69, 134.65, 134.06, 133.71, 133.62, 130.19, 129.76, 129.56, 129.46, 129.01, 128.39, 126.46, 126.34, 126.21, 121.15, 119.98, 118.63, 118.37, 117.79, 87.84, 67.99, 60.45, 56.82, 56.09, 55.89, 54.88, 53.09, 51.75, 44.17, 41.80, 35.37, 34.33, 29.85, 28.90, 27.04, 26.34, 23.51, 19.77, 18.58, 12.71, 11.13.

### Synthesis of (9H-fluoren-9-yl)methyl ((6R,9S,12S,15R)-9-ethyl-6-(4-fluorophenyl)-4,15-dimethyl-5,8,11,14-tetraoxo-12-(3-(3-((2,2,4,6,7-pentamethyl-2,3-dihydrobenzofuran-5-yl)sulfonyl)guanidino)propyl)-4,7,10,13-tetraazaoctadeca-1,17-dien-15-yl)carbamate (35f)

Intermediate 35f was prepared according to Method A starting from 32f. Intermediate 28 was used as the peptide carboxylic acid. The crude product was purified over flash chromatography using ethylacetate. White solid (45% yield). MS (ESI): m/z calc. for [M+H]⁺ 1049.26, found 1049.66. ¹H NMR (300 MHz, CD₃OD, 2-rotamers) δ: 7.80 (d, J=7.5 Hz, 2H), 7.71-7.61 (m, 2H), 7.46-7.26 (m, 6H), 7.10-6.99 (m, 2H), 5.78 (s, 1H), 5.75-5.49 (m, 2H), 5.11-4.96 (m, 4H), 4.61-4.49 (m, 1H), 4.37-4.26 (m, 1H), 4.26-4.13 (m, 3H), 4.05-3.75 (m, 2H), 3.17-3.10 (m, 2H), 2.96 (s, 2H), 2.87 (s, 1.6H), 2.83 (s, 1.4H), 2.63-2.35 (m, 8H), 2.06 (s, 3H), 1.93-1.46 (m, 6H), 1.42 (s, 6H), 1.32 (s, 3H), 0.87 (t, J=7.3 Hz, 3H). ¹³C NMR (75 MHz, CD₃OD, rotamers) δ: 177.06, 176.97, 174.47, 173.57, 173.51, 171.40, 171.17, 165.83, 162.55, 160.02, 158.25, 157.92, 145.46, 145.40, 142.83, 142.81, 139.56, 134.63, 134.03, 133.68, 133.62, 133.57, 131.69, 131.58, 131.49, 129.01, 128.37, 126.43, 126.31, 126.19, 121.16, 120.01, 118.61, 118.30, 117.86, 117.00, 116.94, 116.71, 116.65, 87.81, 68.01, 60.38, 56.85, 55.26, 55.00, 53.06, 51.75, 44.14, 41.68, 35.36, 34.40, 29.73, 28.89, 27.15, 26.29, 26.23, 23.58, 19.80, 18.60, 12.72, 11.21.

### Synthesis of (9H-fluoren-9-yl)methyl ((6R,9S,12S,15R)-6-(4-chlorophenyl)-9-ethyl-4,15-dimethyl-5,8,11,14-tetraoxo-12-(3-(3-((2,2,4,6,7-pentamethyl-2,3-dihydrobenzofuran-5-yl)sulfonyl)guanidino)propyl)-4,7,10,13-tetraazaoctadeca-1,17-dien-15-yl)carbamate (35g)

Intermediate 35g was prepared according to Method A starting from 32g. Intermediate 28 was used as the peptide carboxylic acid. The product was purified over flash chromatography using ethylacetate. White solid (44% yield). MS (ESI): m/z calc. for [M+H]⁺ 1065.47, found 1065.08. ¹H NMR (300 MHz, CD₃OD, 2-rotamers) δ: 7.80 (d, J=7.5 Hz, 2H), 7.69-7.62 (m, 2H), 7.43-7.25 (m, 8H), 5.78 (s, 1H), 5.73-5.50 (m, 2H), 5.12-4.97 (m, 4H), 4.60-4.51 (m, 1H), 4.35-4.27 (m, 1H), 4.25-4.14 (m, 3H), 4.04-3.78 (m, 2H), 3.19-3.09 (m, 2H), 2.96 (s, 2H), 2.90-2.81 (m, 3H), 2.62-2.36 (m, 8H), 2.06 (s, 3H), 1.94-1.46 (m, 6H), 1.42 (s, 6H), 1.32 (s, 3H), 0.88 (t, J=7.3 Hz, 3H). ¹³C NMR (75 MHz, CD₃OD, rotamers) δ: 177.03, 174.50, 173.64, 173.59, 171.18, 170.94, 160.04, 158.27, 157.96, 145.47, 145.42, 142.84, 139.58, 137.09, 136.55, 135.55, 135.46, 134.63, 134.06, 134.04, 133.69, 133.65, 133.57, 131.24, 131.16, 130.22, 130.15, 129.02, 128.38, 126.45, 126.31, 126.20, 121.17, 120.03, 118.62, 118.32, 117.92, 87.82, 68.02, 60.38, 56.91, 55.31, 55.03, 53.09, 51.78, 44.15, 41.66, 35.38, 34.43, 29.71, 28.90, 27.16, 26.28, 26.22, 23.58, 19.80, 18.61, 12.73, 11.24

### Synthesis of (9H-fluoren-9-yl)methyl ((6R,9S,12S,15R)-9-ethyl-15-methyl-12-(3-((E)-2-methyl-3-((2,2,4,6,7-pentamethyl-2,3-dihydrobenzofuran-5-yl)sulfonyl)guanidino)propyl)-5,8,11,14-tetraoxo-6-phenyl-4,7,10,13-tetraazaoctadeca-1,17-dien-15-yl)carbamate (35h)

Intermediate 35h was prepared according to Method A starting from 34h (0.33 g; 0.35 mmol). Intermediate 21 was used as the amino acid. The compound was purified over flash chromatography using EtOAc:MeOH (10:0.1) affording 0.29 g (0.28 mmol) intermediate 35h as white solid (85% yield). MS (ESI): m/z calc. for [M+H]⁺ 1031.51, found 1031.08. ¹H NMR (300 MHz, CD₃OD) δ:7.79 (d, J=7.4 Hz, 2H), 7.65 (d, J=7.4 Hz, 2H), 7.42-7.25 (m, 9H), 5.78-5.60 (m, 2H), 5.40 (s, 1H), 5.12-4.91 (m, 4H), 4.52-4.43 (m, 1H), 4.36-4.15 (m, 4H), 3.84-3.64 (m, 2H), 3.20-3.09 (m, 2H), 2.95 (s, 2H), 2.73 (s, 3H), 2.67-2.40 (m, 8H), 2.05 (s, 3H), 1.95-1.46 (m, 6H), 1.42 (s, 6H), 1.35 (s, 3H), 0.89 (t, J=7.4 Hz, 3H). ¹³C NMR (75 MHz, CD₃OD) δ: 174.56, 174.06, 172.31, 160.00, 158.02, 157.57, 145.42, 145.38, 142.85, 139.46, 138.78, 135.33, 134.83, 134.02, 133.58, 129.94, 129.51, 129.08, 129.04, 128.39, 126.40, 126.35, 126.22, 121.18, 120.04, 118.65, 116.32, 87.83, 68.08, 60.42, 59.35, 57.02, 44.16, 42.91, 42.10, 41.87, 28.91, 28.70, 25.97, 23.58, 19.86, 18.66, 12.72, 11.16.

### Synthesis of (R)—N-((6S,9S,12R)-9-ethyl-1-imino-7,10,13-trioxo-1-(2,2,4,6,7-pentamethyl-2,3-dihydrobenzofuran-5-sulfonamido)-12-phenyl-2,8,11,14-tetraazaheptadec-16-en-6-yl)-2-isobutyramido-2-methylpent-4-enamide (36a)

Intermediate 35a (0.43 mmol, 0.44 g) was dissolved in 50 mL CH₃CN and treated with 20 equiv. diethylamine (8.6 mmol, 0.63 mL) at room temperature for 2h. The solvent and diethylamine were removed in vacuo and the resulting crude product was mixed with isobutyryl chloride (1.9 mmol, 0.2 mL) and DIPEA (1.9 mmol, 0.33 mL) in CH₂Cl₂ at room temperature for 2h. The reaction mixture was evaporated and the remaining crude product was purified over flash chromatography using EtAc:MeOH (50:0.7) yielding 0.23 g intermediate (36a) as white solid (62% yield). HRMS (ESI): m/z calc. for C₄₄H₆₅N₈O₈S [M+H]⁺ 865.4641, found 865.4642. ¹H NMR (300 MHz, CD₃OD) δ: 7.45-7.28 (m, 5H), 5.84-5.64 (m, 2H), 5.42 (s, 1H), 5.14-4.99 (m, 4H), 4.26-4.19 (m, 2H), 3.89-3.69 (m, 2H), 3.15 (t, J=6.7 Hz, 2H), 2.99 (s, 2H), 2.74-2.65 (m, 1H), 2.60-2.40 (m, 8H), 2.07 (s, 3H), 1.98-1.48 (m, 6H), 1.45 (s, 6H), 1.37 (s, 3H), 1.13-1.06 (m, 6H), 0.92 (t, J=7.4 Hz, 3H). ¹³C NMR (75 MHz, CD₃OD): δ 180.33, 176.96, 174.56, 174.22, 172.49, 160.08, 158.33, 139.58, 138.59, 135.36, 134.61, 134.16, 133.72, 129.93, 129.55, 129.21, 126.22, 119.95, 118.64, 116.31, 87.87, 59.99, 59.54, 56.93, 55.03, 44.19, 42.92, 41.24, 36.15, 30.01, 28.91, 25.96, 23.42, 20.43, 20.03, 19.84, 19.75, 18.57, 12.69, 11.25.

### Synthesis of (R)—N-((6S,9S,12R)-9-ethyl-1-imino-7,10,13-trioxo-1-(2,2,4,6,7-pentamethyl-2,3-dihydrobenzofuran-5-sulfonamido)-12-phenyl-2,8,11,14-tetraazaoctadec-17-en-6-yl)-2-isobutyramido-2-methylpent-4-enamide (36b)

Intermediate 36b was prepared according to Method A starting from 32b. Intermediate 26 was used as the peptide carboxylic acid. The crude product was purified over flash chromatography using EtOAc:MeOH (30:1). White solid (72% yield). MS (ESI): m/z calc. for [M+H]⁺ 879.48, found 879.42. ¹H NMR (300 MHz, CD₃OD) δ: 7.44-7.23 (m, 5H), 5.80-5.63 (m, 2H), 5.40 (s, 1H), 5.15-4.91 (m, 4H), 4.27-4.17 (m, 2H), 3.28-3.09 (m, 4H), 2.99 (s, 2H), 2.76-2.65 (m, 1H), 2.61-2.40 (m, 8H), 2.21 (q, J=6.9 Hz, 2H), 2.07 (s, 3H), 1.97-1.48 (m, 6H), 1.44 (s, 6H), 1.37 (s, 3H), 1.15-1.04 (m, 6H), 0.92 (t, J=7.4 Hz, 3H). ¹³C NMR (75 MHz, CD₃OD) δ: 180.29, 176.98, 174.53, 174.16, 172.47, 160.03, 158.24, 139.55, 138.53, 136.56, 134.55, 134.14, 133.67, 129.86, 129.48, 129.21, 126.18, 119.99, 118.60, 117.44, 87.83, 59.89, 59.47, 56.90, 55.09, 44.16, 41.66, 41.17, 40.27, 36.08, 34.86, 29.88, 28.94, 27.12, 25.95, 23.42, 20.51, 19.83, 18.64, 12.76, 11.32.

### Synthesis of (9H-fluoren-9-yl)methyl ((4R,7S,10S,13R)-10-ethyl-4-methyl-5,8,11,14-tetraoxo-7-(3-(3-((2,2,4,6,7-pentamethyl-2,3-dihydrobenzofuran-5-yl)sulfonyl)guanidino)propyl)-13-phenyl-6,9,12,15-tetraazaicosa-1,19-dien-4-yl)carbamate (36c)

Intermediate 36c was prepared according to Method A starting from 32c. Intermediate 26 was used as the peptide carboxylic acid. The crude product was purified over flash chromatography using EtOAc:MeOH (25:4) White solid (80% yield). MS (ESI): m/z calc. for [M+H]⁺ 893.50, found 893.62. ¹H NMR (300 MHz, CD₃OD) δ: 7.43-7.26 (m, 5H), 5.84-5.64 (m, 2H), 5.38 (s, 1H), 5.14-4.91 (m, 4H), 4.26-4.18 (m, 2H), 3.28-3.06 (m, 4H), 2.99 (s, 2H), 2.75-2.64 (m, 1H), 2.61-2.41 (m, 8H), 2.08 (s, 3H), 2.05-1.48 (m, 10H), 1.45 (s, 6H), 1.37 (s, 3H), 1.14-1.06 (m, 6H), 0.92 (t, J=7.4 Hz, 3H). ¹³C NMR (75 MHz, CD₃OD) δ: 180.31, 176.94, 174.56, 174.18, 172.50, 160.08, 158.33, 139.59, 139.24, 138.69, 134.61, 134.14, 133.72, 129.91, 129.51, 129.18, 126.22, 119.96, 118.64, 115.72, 87.87, 59.99, 59.51, 56.96, 50.55, 44.20, 41.28, 40.34, 36.15, 32.24, 30.00, 29.92, 28.91, 27.04, 25.97, 23.40, 20.45, 19.85, 19.76, 18.58, 12.69, 11.26.

### Synthesis of (R)—N-((6S,9S,12R)-9-ethyl-1-imino-7,10,13-trioxo-1-(2,2,4,6,7-pentamethyl-2,3-dihydrobenzofuran-5-sulfonamido)-12-phenyl-2,8,11,14-tetraazahenicos-20-en-6-yl)-2-isobutyramido-2-methylpent-4-enamide (36d)

Intermediate 36d was prepared according to Method A starting from 32d. Intermediate 26 was used as the peptide carboxylic acid. The crude product was purified over flash chromatography using EtOAc:MeOH (40:1) to afford 36d as white solid (71% yield). MS (ESI): m/z calc. for [M+H]⁺ 921.53, found 921.42. ¹H NMR (300 MHz, CD₃OD) δ: 7.43-7.28 (m, 5H), 5.83-5.64 (m, 2H), 5.39 (s, 1H), 5.14-4.90 (m, 4H), 4.25-4.19 (m, 2H), 3.26-3.07 (m, 4H), 2.99 (s, 2H), 2.74-2.65 (m, 1H), 2.58-2.41 (m, 8H), 2.07 (s, 3H), 2.04-1.46 (m, 10H), 1.45 (s, 6H), 1.37 (s, 3H), 1.35-1.21 (m, 4H), 1.13-1.08 (m, 6H), 0.92 (t, J=7.4 Hz, 3H). ¹³C NMR (75 MHz, CD₃OD) δ: 180.32, 177.00, 174.56, 174.15, 172.45, 160.06, 158.29, 140.09, 139.57, 138.66, 134.57, 134.16, 133.70, 129.89, 129.49, 129.18, 126.20, 119.98, 118.62, 115.10, 87.85, 59.93, 59.50, 56.90, 50.10, 44.18, 41.70, 41.22, 40.73, 36.12, 34.97, 30.46, 29.94, 28.92, 27.54, 27.08, 25.97, 23.40, 20.49, 19.83, 19.79, 18.61, 12.72, 11.30.

### Synthesis of (R)—N-((6S,9S,12R)-9-ethyl-1-imino-14-methyl-7,10,13-trioxo-1-(2,2,4,6,7-pentamethyl-2,3-dihydrobenzofuran-5-sulfonamido)-12-phenyl-2,8,11,14-tetraazaheptadec-16-en-6-yl)-2-isobutyramido-2-methylpent-4-enamide (36e)

Intermediate 36e was prepared starting from 35e according to the procedure used for 36a. The reaction mixture was concentrated under vacuo and the remaining crude product was purified over flash chromatography using EtAc:MeOH (20:1) yielding 0.19 g intermediate 36e as white solid (45% yield). MS (ESI): m/z calc. for [M+H]⁺ 879.48, found 879.78. ¹H NMR (300 MHz, CD₃OD, 2-rotamers) δ: 7.44-7.27 (m, 5H), 5.80 (s, 1H), 5.76-5.47 (m, 2H), 5.16-4.99 (m, 4H), 4.31-4.18 (m, 2H), 4.07-3.81 (m, 2H), 3.19-3.10 (m, 2H), 3.00 (s, 2H), 2.90 (s, 1.6H), 2.87 (s, 1.4H),2.69-2.38 (m, 9H), 2.08 (s, 3H), 1.92-1.47 (m, 6H) 1.45 (s, 6H), 1.36 (s, 3H), 1.09 (d, J=6.8 Hz, 6H), 0.90 (t, J=7.4 Hz, 3H). ¹³C NMR (75 MHz, CD₃OD, rotamers) δ: 180.00, 176.66, 176.55, 174.35, 174.29, 173.66, 171.65, 171.46, 160.06, 158.31, 139.59, 138.21, 137.63, 134.65, 134.19, 133.72, 133.64, 130.18, 130.14, 129.79, 129.70, 129.62, 129.52, 126.20, 119.87, 118.62, 118.39, 117.78, 87.86, 60.06, 56.84, 56.16, 55.93, 54.84, 54.73, 53.15, 51.81, 44.19, 41.76, 41.30, 36.15, 35.42, 34.37, 30.20, 28.91, 26.81, 26.36, 26.27, 23.36, 20.41, 20.38, 19.85, 19.77, 18.59, 12.70, 11.24.

### Synthesis of (R)—N-((6S,9S,12R)-9-ethyl-12-(4-fluorophenyl)-1-imino-14-methyl-7,10,13-trioxo-1-(2,2,4,6,7-pentamethyl-2,3-dihydrobenzofuran-5-sulfonamido)-2,8,11,14-tetraazaheptadec-16-en-6-yl)-2-isobutyramido-2-methylpent-4-enamide (36f)

Intermediate 36f was prepared starting from 35f according to the procedure used for 36a. The compound was purified over flash chromatography using ethylacetate:MeOH (15:1). White solid (58% yield). MS (ESI): m/z calc. for [M+H]⁺ 897.47, found 897.76. ¹H NMR (300 MHz, CD₃OD, 2-rotamers) δ: 7.47-7.38 (m, 2H), 7.11-7.01 (m, 2H), 5.81 (s, 1H), 5.77-5.61 (m, 2H), 5.16-5.01 (m, 4H), 4.27-4.17 (m, 2H), 4.09-3.81 (m, 2H), 3.21-3.11 (m, 2H), 3.00 (s, 2H), 2.92 (s, 1.6H), 2.87 (s, 1.4H), 2.68-2.37 (m, 9H), 2.08 (s, 3H), 1.93-1.43 (m, 12H), 1.35 (s, 3H), 1.09 (d, J=6.8 Hz, 6H), 0.90 (t, J=7.3 Hz, 3H). ¹³C NMR (75 MHz, CD₃OD, rotamers) δ: 180.05, 176.88, 176.73, 174.45, 174.37, 173.74, 171.50, 171.30, 165.89, 162.62, 160.06, 158.31, 139.57, 134.62, 134.23, 134.18, 133.83, 133.70, 133.62, 131.78, 131.68, 131.57, 126.20, 119.89, 118.62, 118.35, 117.85, 116.99, 116.89, 116.70, 116.60, 87.85, 59.98, 59.96, 56.96, 56.92, 55.35, 55.06, 53.16, 51.83, 44.18, 41.73, 41.10, 36.12, 35.41, 34.43, 30.04, 28.91, 26.94, 26.33, 26.22, 23.42, 20.48, 20.45, 19.82, 19.77, 18.59, 12.70, 11.35, 11.28.

### Synthesis of (R)—N-((6S,9S,12R)-12-(4-chlorophenyl)-9-ethyl-1-imino-14-methyl-7,10,13-trioxo-1-(2,2,4,6,7-pentamethyl-2,3-dihydrobenzofuran-5-sulfonamido)-2,8,11,14-tetraazaheptadec-16-en-6-yl)-2-isobutyramido-2-methylpent-4-enamide (36g)

Intermediate 36g was prepared starting from 35g according to the procedure used for 36a. The compound was purified over flash chromatography using ethylacetate:MeOH (70:1). White solid (76% yield). MS (ESI): m/z calc. for [M+H]⁺ 913.44, found 913.42. ¹H NMR (300 MHz, CD₃OD, 2-rotamers) δ: 7.45-7.29 (m, 4H), 5.81 (s, 1H), 5.77-5.62 (m, 2H), 5.17-5.02 (m, 4H), 4.27-4.16 (m, 2H), 4.09-3.87 (m, 2H), 3.19-3.12 (m, 2H), 3.00 (s, 2H), 2.92 (s, 1.7H), 2.87 (s, 1.3H), 2.67-2.38 (m, 9H), 2.08 (s, 3H), 1.92-1.43 (m, 12H), 1.35 (m, 3H), 1.09 (d, J=6.9 Hz, 6H), 0.91 (t, J=7.4 Hz, 3H). ¹³C NMR (75 MHz, CD₃OD, rotamers) δ: 180.05, 176.98, 176.79, 174.52, 174.43, 173.77, 171.25, 171.04, 160.03, 158.26, 139.54, 137.07, 136.47, 135.53, 135.40, 134.60, 134.20, 134.15, 133.82, 133.67, 133.57, 131.30, 131.22, 130.18, 130.08, 126.18, 119.94, 118.60, 118.40, 117.94, 87.83, 59.93, 57.00, 55.38, 55.23, 55.05, 53.17, 51.84, 44.17, 41.70, 41.04, 40.92, 36.08, 35.43, 34.46, 29.93, 29.86, 28.93, 27.00, 26.27, 26.14, 23.43, 20.52, 20.47, 19.80, 18.61, 12.74, 11.42, 11.34.

### Synthesis of (R)—N-((8S,11S,14R,E)-11-ethyl-9,12,15-trioxo-3-(2,2,4,6,7-pentamethyl-2,3-dihydrobenzofuran-5-sulfonamido)-14-phenyl-2,4,10,13,16-pentaazanonadeca-2,18-dien-8-yl)-2-isobutyramido-2-methylpent-4-enamide (36h)

Intermediate 36h was prepared starting from 35h according to the procedure used for 36a. The compound was purified over flash chromatography using EtOAc:MeOH (250:4). White solid (59% yield). MS (ESI): m/z calc. for [M+H]⁺ 879.48, found 879.50. ¹H NMR (300 MHz, CD₃OD) δ: 7.45-7.40 (m, 2H), 7.37-7.25 (m, 3H), 5.85-5.64 (m, 2H), 5.45 (s, 1H), 5.14-4.99 (m, 4H), 4.27-4.20 (m, 2H), 3.90-3.69 (m, 2H), 3.21-3.12 (m, 2H), 2.98 (s, 2H), 2.75 (s, 3H), 2.73-2.2.65 (m, 1H), 2.58 (s, 3H), 2.56-2.41 (m, 5H), 2.07 (s, 3H), 2.00-1.49 (m, 6H), 1.44 (s, 6H), 1.37 (s, 3H), 1.14-1.06 (m, 6H), 0.93 (t, J=7.4 Hz, 3H). ¹³C NMR (75 MHz, CD₃OD) δ:180.28, 176.97, 174.49, 174.12, 172.41, 159.96, 157.51, 139.39, 138.53, 135.33, 134.83, 134.15, 133.52, 129.88, 129.49, 129.17, 126.17, 119.95, 118.60, 116.28, 87.81, 59.89, 59.50, 56.89, 55.01, 44.15, 42.86, 42.07, 41.14, 36.07, 29.88, 28.94, 28.72, 27.11, 25.93, 23.46, 20.49, 19.87, 19.82, 18.67, 12.74, 11.30.

### Synthesis of (R)—N-((8S,11S,14R,Z)-11-ethyl-3-(methylamino)-9,12,15-trioxo-14-phenyl-2,4,10,13,16-pentaazanonadeca-2,18-dien-8-yl)-2-isobutyramido-2-methylpent-4-enamide (36i)

Intermediate 36i was prepared according to Method A starting from 32a. Intermediate 30 was used as the peptide carboxylic acid. The crude product was purified with preparative HPLC using the C18 reverse phase column (Waters, Sunfire™ Prep Cis OBD™, 5 μm, 50×100 mm). White solid (44% yield). MS (ESI): m/z calc. for [M+H]⁺ 641.41, found 641.42. ¹H NMR (300 MHz, CD₃OD) δ: 7.45-7.28 (m, 5H), 5.87-5.65 (m, 2H), 5.42 (s, 1H), 5.16-5.01 (m, 4H), 4.27-4.16 (m, 2H), 4.32 (dd, J=5.0, 7.6 Hz, 1H), 4.25 (dd, J=5.7, 8.8 Hz, 1H), 3.90-3.72 (m, 2H), 3.20 (t, J=7.0 Hz, 2H), 2.83 (s, 6H), 2.74-2.64 (m, 1H), 2.60-2.41 (m, 2H), 1.99-1.57 (m, 6H), 1.39 (m, 3H), 1.15-1.07 (m, 6H), 0.93 (t, J=7.4 Hz, 3H). ³C NMR (75 MHz, CD₃OD) δ: 180.33, 176.81, 174.35, 174.13, 172.46, 157.45, 138.61, 135.35, 133.98, 129.94, 129.58, 129.15, 119.97, 116.33, 60.05, 59.51, 56.86, 54.42, 42.92, 42.13, 41.69, 36.07, 30.19, 28.50, 26.17, 26.04, 23.19, 20.38, 19.79, 11.12.

### Synthesis of (R)-2-((((9H-fluoren-9-yl)methoxy)carbonyl)amino)-4-(dibenzylamino)-2-methylbutanoic acid CF₃COOH salt (40)

(R)-2-Fmoc-NH-2-methylpent-4-enoic acid (1 mmol, 350 mg) was dissolved in THF/H₂O (2.6 mL/1 mL) in a flask. The hood lights were turned off and the flask was covered by aluminum foil. Osmium tetroxide (0.05 mmol in 0.32 mL H₂O) was added to the flask. After 5 min, NaIO₄ (2.5 mmol, 535 mg) was added in small portions over a 15 min period. The reaction was kept at room temperature for 4 h before it was filtrated and the THF was evaporated off. The residue was dissolved in EtOAc and saturated NH₄C aqueous solution was added. The aqueous phase was extracted with EtOAc (2×30 mL) and the organic phase was combined, washed with brine, and dried over anhydrous Na₂SO₄. The solution was concentrated in vacuo and the crude product was dissolved in 1,2-dichloroethane (3 mL) in a flask. Then dibenzylamine (1.5 mmol, 0.29 mL) and sodium triacetoxyborohydride (3 mmol, 636 mg) were added to the flask. The reaction was stirred at room temperature until the starting material disappeared on TLC. The solvent was evaporated and the remaining crude product was purified with C-18 reverse phase flash column to yield 40. White solid (75% yield over 2 steps) MS (ESI): m/z calculated for C₃₄H₃₅N₂O₄ [M+H]⁺ 535.26, found 535.23. ¹H NMR (400 MHz, Methanol-d₄) δ 7.80 (d, J=7.6 Hz, 2H), 7.62 (dd, J=15.7, 7.5 Hz, 2H), 7.48-7.35 (m, 12H), 7.33-7.25 (m, 2H), 4.43-4.05 (m, 7H), 3.25-3.03 (m, 2H), 2.55 (q, J=8.3, 6.5 Hz, 1H), 2.46-2.20 (m, 1H), 1.35 (s, 3H). ¹³C NMR (101 MHz, MeOD) δ 176.68, 157.48, 145.26, 145.07, 142.58, 142.56, 132.20, 131.24, 130.45, 130.44, 128.86, 128.19, 126.29, 126.14, 120.98, 67.91, 58.81, 58.40, 48.28, 31.28, 24.16.

Method B. To a solution of intermediate 36a-36h in CH₂Cl₂, Hoveyda-Grubbs 2^{nd} Generation catalyst (0.5 equiv.) was added under N₂ atmosphere, and the reaction mixture stirred at 45° C. overnight under N₂ atmosphere unless stated otherwise. With intermediates 36e-36g another portion of the catalyst (0.5 equiv.) was added and stirred further overnight under the same conditions before filtering through celite and concentrating in vacuo. The remaining crude product was purified over flash chromatography using CH₂Cl₂:MeOH. The cyclic product was taken up to MeOH and the double bond was reduced using 10% Pd/C under 1 atm of H₂. The reaction mixture was filtered through celite and concentrated. The remaining crude product was refluxed in CH₂Cl₂:Trifluoroacetic acid:H₂O (20:10:0.5) for 2h in order to remove the Pbf group from arginine guanidine, and evaporated. The crude product was purified with preparative HPLC using the C18 reverse phase column (Waters, Sunfire™ Prep C18 OBD™, 5 μm, 50×100 mm). The final compound then dissolved in CH₃CN:H₂O (1:1) and lyophilized

### Synthesis of N-((3R,6S,9S,12R)-6-ethyl-9-(3-guanidinopropyl)-12-methyl-2,5,8,11-tetraoxo-3-phenyl-1,4,7,10-tetraazacyclohexadecan-12-yl)isobutyramide (Cpd. No. 64) CF₃COOH salt

Cpd. No. 64 was prepared according Method B starting from 36a. The RCM cyclization was achieved at room temperature overnight. White solid (53% yield over 3 steps). HRMS (ESI+): m/z calculated for C₂₉H₄₇N₈O₅ [M+H]⁺ 587.3664, found 587.3664. ¹H NMR (300 MHz, MeOD): δ 7.42-7.30 (m, 5H), 5.25 (s, 1H), 4.30 (dd, J=4.2, 9.8 Hz, 1H), 4.16 (dd, J=6.2, 7.7 Hz, 1H), 3.51-3.42 (m, 1H), 3.19 (t, J=6.9 Hz, 2H), 3.12-2.99 (m, 1H), 2.59-2.48 (m, 1H), 1.94-1.26 (m, 15H), 1.15-1.08 (m, 6H), 0.92 (t, J=7.4 Hz, 3H). ¹³C NMR (75 MHz, MeOD-d₄): δ 179.86, 176.83, 174.99, 173.64, 172.97, 158.86, 137.93, 130.06, 129.69, 129.32, 61.24, 60.70, 56.67, 55.27, 41.91, 40.34, 39.25, 36.12, 29.99, 26.39, 25.94, 22.81, 21.83, 20.06, 19.91, 10.84.

### Synthesis of N-((3R,6S,9S,12R)-6-ethyl-9-(3-guanidinopropyl)-1,12-dimethyl-2,5,8,11-tetraoxo-3-phenyl-1,4,7,10-tetraazacyclohexadecan-12-yl)isobutyramide (Cpd. No. 65) CF₃COOH salt

The compound was prepared according Method B starting from 36e. White solid (18% yield over 3 steps). HRMS (ESI): m/z calc. for C₃₀H₄₉N₈O₅ [M+H]⁺ 601.3820, found 601.3819. ¹H NMR (300 MHz, CD₃OD, rotamers) δ: 7.41-7.21 (m, 5H), 5.91 (s, 1H), 4.28-4.20 (m, 1H), 4.10 (dd, J=4.7, 10.1 Hz, 1H), 3.21 (t, J=7.1 Hz, 2H), 3.01 (s, 2.6H),2.95 (s, 0.4H), 2.84-2.72 (m, 1H), 2.62-2.51 (m, 1H), 2.07-1.21 (m, 16H), 1.18-1.08 (m, 6H), 1.00 (t, J=7.3 Hz, 2.4H), 0.91 (t, J=7.3 Hz, 0.6H). ¹³C NMR (75 MHz, CD₃OD, rotamers) δ: 180.14, 176.79, 174.99, 173.94, 172.30, 158.88, 138.57, 129.49, 129.28, 128.95, 60.76, 57.29, 56.26, 56.00, 47.38, 42.01, 40.82, 36.21, 35.54, 29.60, 27.91, 26.38, 25.61, 25.50, 21.67, 20.58, 19.72, 17.73.

### Synthesis of N-((3R,6S,9S,12R)-6-ethyl-3-(4-fluorophenyl)-9-(3-guanidinopropyl)-1,12-dimethyl-2,5,8,11-tetraoxo-1,4,7,10-tetraazacyclohexadecan-12-yl)isobutyramide (Cpd. No. 66) CF₃COOH salt

The compound was synthesized using Method B starting from 36f. White solid (11% yield over 3 steps). HRMS (ESI): m/z calculated for C₃₀H₄₈FN₈O₅ [M+H]⁺ 619.3726, found 619.3731. ¹H NMR (300 MHz, CD₃OD, 2-rotamers) δ: 7.44-7.36 (m, 2H), 7.16-7.00 (m, 2H), 5.94 (s, 1H), 4.24-4.16 (m, 1H), 4.07 (dd, J=4.2, 10.5 Hz, 1H), 3.20 (t, J=7.1 Hz, 2H), 2.99 (s, 2.5H), 2.93 (s, 0.5 Hz), 2.78-2.68 (m, 1H), 2.63-2.49 (m, 1H), 2.14-1.18 (m, 16H), 1.15 (d, J=6.8 Hz, 3H), 1.11 (d, J=6.7 Hz, 3H), 1.01 (t, J=7.3 Hz, 2.5H), 0.89 (t, J=7.4 Hz, 0.5H). ¹³C NMR (75 MHz, CD₃OD) δ: 180.14, 177.04, 175.19, 174.05, 172.07, 158.88, 134.96, 134.93, 131.36, 131.26, 116.12, 115.83, 60.69, 57.55, 56.61, 55.15, 46.99, 42.01, 40.67, 36.21, 35.29, 29.36, 27.51, 26.43, 26.16, 25.44, 21.54, 20.72, 19.63, 11.89.

### Synthesis of N-((3R,6S,9S,12R)-3-(4-chlorophenyl)-6-ethyl-9-(3-guanidinopropyl)-1,12-dimethyl-2,5,8,11-tetraoxo-1,4,7,10-tetraazacyclohexadecan-12-yl)isobutyramide (Cpd. No. 67) CF₃COOH salt (Cpd. No. 67)

The compound was synthesized using Method B starting from 36g. Ph₂S was added during catalytic hydrogenation in order to reduce the activity of Pd/C. White solid (11% yield over 3 steps). HRMS (ESI): m/z calculated for C₃₀H₄₈ClN₈O₅ [M+H]⁺ 635.3431, found 635.3428. ¹H NMR (300 MHz, CD₃OD, rotamers) δ: 7.41-7.29 (m, 4H), 5.95 (s, 1H), 4.20 (t, 1H, J=6.6 Hz), 4.07 (dd, 1H, J=4.2, 10.4 Hz), 3.25-3.17 (m, 2H), 2.99 (s, 2.4H), 2.94 (s, 0.4H), 2.90 (s, 0.2H), 2.77-2.68 (m, 1H), 2.62-2.51 (m, 1H), 2.15-1.59 (m, 9H), 1.55-1.18 (m, 7H), 1.16 (d, 3H, J=6.8 Hz), 1.12 (d, 3H, J=6.7 Hz), 1.02 (t, J=7.4 Hz, 2.3H), 0.90 (t, J=7.5 Hz, 0.7H). ¹³C NMR (75 MHz, CD₃OD) δ: 180.13, 177.03, 175.22, 174.10, 171.85, 158.89, 137.86, 134.64, 131.07, 129.38, 60.68, 57.64, 56.59, 55.23, 46.88, 42.01, 40.69, 36.23, 35.25, 29.35, 27.43, 26.43, 26.29, 25.41, 21.51, 20.75, 19.61, 11.92.

### Synthesis of N-((3R,6S,9S,12R)-6-ethyl-12-methyl-9-(3-(3-methylguanidino)propyl)-2,5,8,11-tetraoxo-3-phenyl-1,4,7,10-tetraazacyclohexadecan-12-yl)isobutyramide (Cpd. No. 68) CF₃COOH salt

The compound was synthesized using Method B starting from 36h. The RCM cyclization was achieved at room temperature overnight. White solid (75% yield over 3 steps). HRMS (ESI): m/z calculated for C₃₀H₄₉N₈O₅ [M+H]⁺ 601.3820, found 601.3829. ¹H NMR (300 MHz, CD₃OD) δ: 7.42-7.33 (m, 5H), 5.24 (s, 1H), 4.30 (dd, J=4.2, 9.8 Hz, 1H), 4.17 (dd, J=6.0, 7.8 Hz, 1H), 3.52-3.43 (m, 1H), 3.18 (t, J=6.9 Hz, 2H), 3.11-2.99 (m, 1H), 2.85 (s, 3H), 2.59-2.48 (m, 1H), 1.96-1.24 (m, 15H), 1.18-1.06 (m, 6H), 0.92 (t, J=7.5 Hz, 3H). ¹³C NMR (75 MHz, CD₃OD) δ: 179.84, 176.79, 175.01, 173.94, 172.98, 158.42, 137.90, 130.06, 129.70, 129.33, 61.29, 60.68, 56.62, 55.22, 41.93, 40.39, 39.28, 36.11, 30.06, 28.54, 26.40, 25.98, 22.79, 21.86, 20.08, 19.90, 10.81.

### Synthesis of N-((3R,6S,9S,12R)-9-(3-((E)-2,3-dimethylguanidino)propyl)-6-ethyl-12-methyl-2,5,8,11-tetraoxo-3-phenyl-1,4,7,10-tetraazacyclohexadecan-12-yl)isobutyramide (Cpd. No. 69) CF₃COOH salt

The compound was synthesized according to Method B starting from 36i with the following modification. The RCM cyclization was achieved at room temperature overnight. After the catalytic hydrogenation step the crude product was taken into CH₂Cl₂:Trifluoroacetic acid (1:1) and stirred for 0.5 h at room temperature in order to form the trifluoroacetic acid salt. This mixture was evaporated and purified. White solid (27% yield over 3 steps). HRMS (ESI): m/z calculated for C₃₁H₅₁N₈O₅ [M+H]⁺ 615.3977, found 615.3975. ¹H NMR (300 MHz, CD₃OD) δ: 7.42-7.33 (m, 5H), 5.24 (s, 1H), 4.36-4.28 (m, 1H), 4.18 (dd, J=6.0, 7.8 Hz, 1H), 3.53-3.43 (m, 1H), 3.20 (t, J=7.0 Hz, 2H), 3.10-2.98 (m, 1H), 2.85 (s, 6H), 2.59-2.48 (m, 1H), 1.96-1.23 (m, 15H), 1.15-1.07 (m, 6H), 0.92 (t, J=7.4 Hz, 3H). ¹³C NMR (75 MHz, CD₃OD) δ: 179.79, 176.72, 175.06, 173.95, 172.97, 157.53, 137.88, 130.06, 129.71, 129.32, 61.34, 60.66, 56.56, 55.17, 41.93, 40.41, 39.29, 36.10, 30.06, 28.52, 26.34, 26.01, 22.89, 21.91, 20.07, 19.91, 10.81.

### Synthesis of N-((3S,6R,9R,12S)-6-ethyl-9-(3-guanidinopropyl)-12-methyl-2,5,8,11-tetraoxo-3-phenyl-1,4,7,10-tetraazacyclohexadecan-12-yl)isobutyramide (Cpd. No. 70) CF₃COOH salt

The procedure, used to make Cpd. No. 64, was applied for making Cpd. No. 70. Identical NMR spectrum, ESI-MS data were observed.

### Synthesis of N-((3R,6S,9S,12R)-6-ethyl-9-(3-guanidinopropyl)-2,5,8,11-tetraoxo-3-phenyl-1,4,7,10-tetraazacyclotridecan-12-yl)isobutyramide (Cpd. No. 71) CF₃COOH salt

A method applied for 12 was used to make 9 starting from 24. White solid (24.5% yield over 4 steps). HRMS (ESI): m/z calculated for C₂₅H₃₉N₈O₅ [M+H]⁺ 531.3038, found 531.3040. ¹H NMR (400 MHz, DMSO-d₆) δ 7.47-7.12 (m, 5H), 5.29 (s, 1H), 4.36 (t, J=4.4 Hz, 1H), 4.10 (t, J=7.8 Hz, 1H), 4.04 (t, J=7.5 Hz, 1H), 3.58 (dd, J=13.8, 4.4 Hz, 1H), 3.34 (dd, J=13.6, 4.7 Hz, 1H), 3.12-2.93 (m, 2H), 2.48-2.41 (m, 1H), 1.80-1.29 (m, 6H), 1.03 (d, J=2.6 Hz, 3H), 1.01 (d, J=2.7 Hz, 3H), 0.77 (t, J=7.4 Hz, 3H). ¹³C NMR (75 MHz, DMSO) δ 176.36, 172.67, 171.06, 170.69, 170.63, 156.74, 137.19, 128.18, 127.59, 127.55, 57.49, 55.18, 54.67, 53.39, 40.78, 34.24, 27.40, 25.31, 23.21, 19.48, 19.09, 10.54.

### Synthesis of N-((3R,6S,9S,12R)-6-ethyl-9-(3-guanidinopropyl)-2,5,8,11-tetraoxo-3-phenyl-1,4,7,10-tetraazacyclotetradecan-12-yl)isobutyramide (Cpd. No. 1) CF₃COOH salt

A method applied for Cpd. No. 73 was used to make Cpd. No. 1 starting from 24. White solid (21.7% yield over 4 steps). HRMS (ESI): m/z calculated for C₂₆H₄₁N₈O₅ [M+H]⁺ 545.3200, found 545.3197. ¹H NMR (400 MHz, CD₃OD) δ 7.44-7.36 (m, 2H), 7.36-7.26 (m, 3H), 5.44 (s, 1H), 4.33-4.21 (m, 2H), 4.18 (dd, J=7.7, 6.2 Hz, 1H), 3.68 (dt, J=14.4, 3.7 Hz, 1H), 3.26-3.10 (m, 2H), 2.88 (dd, J=14.3, 11.8 Hz, 1H), 2.65 (hept, J=6.6 Hz, 1H), 2.55-2.39 (m, 1H), 2.22-2.03 (m, 1H), 1.89-1.59 (m, 6H), 1.19 (dd, J=13.8, 6.8 Hz, 6H), 0.94 (t, J=7.4 Hz, 3H). ¹³C NMR (101 MHz, CD₃OD) δ 181.84, 175.27, 175.05, 172.50, 172.27, 158.65, 139.13, 129.49, 129.39, 128.96, 58.55, 57.07, 55.76, 54.13, 41.74, 36.09, 35.68, 30.21, 28.78, 26.53, 24.52, 20.38, 19.42, 11.07.

### Synthesis of N-((3R,6S,9S,12R)-6-ethyl-9-(3-guanidinopropyl)-2,5,8,11-tetraoxo-3-phenyl-1,4,7,10-tetraazacyclopentadecan-12-yl)isobutyramide (Cpd. No. 72) CF₃COOH salt

A method applied for Cpd. No. 73 was used to make Cpd. No. 72 starting from 24. White solid (21.5% yield over 4 steps). HRMS (ESI): m/z calculated for C₂₇H₄₃N₈O₅ [M+H]⁺ 559.3356, found 559.3351. ¹H NMR (400 MHz, CD₃OD) δ 7.47-7.25 (m, 5H), 5.27 (s, 1H), 4.31 (dd, J=10.5, 5.0 Hz, 1H), 4.20 (dd, J=7.9, 6.4 Hz, 1H), 3.96 (dd, J=9.5, 3.7 Hz, 1H), 3.46-3.37 (m, 1H), 3.24-3.01 (m, 3H), 2.65-2.49 (m, 1H), 1.93-1.60 (m, 10H), 1.15 (dd, J=6.9, 5.9 Hz, 6H), 0.93 (t, J=7.5 Hz, 3H). ¹³C NMR (101 MHz, CD₃OD) δ 180.68, 175.30, 174.76, 173.75, 172.98, 158.61, 137.32, 129.90, 129.58, 129.22, 60.94, 56.96, 56.23, 55.33, 41.56, 39.56, 35.76, 29.84, 28.93, 26.58, 26.15, 19.96, 19.63, 10.67.

### Synthesis of N-((3R,6S,9S,12R)-6-ethyl-9-(3-guanidinopropyl)-2,5,8,11-tetraoxo-3-phenyl-1,4,7,10-tetraazacyclohexadecan-12-yl)isobutyramide (Cpd. No. 73) CF₃COOH salt

Fmoc-D-Phg-OH (0.5 mmol, 0.17 g) was loaded on the 0.1 mmol 2-chlorotrityl chloride (24) resin (ChemPep) (1 mmol/g) overnight in CH₂Cl₂ and in the presence of 2,4,6-collidine (3 mmol, 0.4 mL). Then, the resin was washed with DMF, MeOH, CH₂Cl₂, respectively, mixed with DIPEA (0.29 mmol, 0.5 mL) in MeOH:CH₂Cl₂ (1:5) and was shaken for 30 min to endcap unreacted 2-chlorotrityl group on the resin. Next, classical chain elongation was carried out with Fmoc chemistry. The peptide intermediate (38d) was cleaved from the resin by treatment of 37d with 4 ml of 1% trifluoroacetic acid in CH₂Cl₂ (3×10 min). The filtrate was evaporated and followed by treatment with 10% trifluoroacetic acid in CH₂Cl₂ for 30 min. Then the solvent was evaporated and the remaining crude product was purified with preparative HPLC using the C18 reverse phase column (Waters, Sunfire™ Prep C18 OBD™, 5 μm, 50×100 mm) to yield 38d. White powder, MS (ESI): m/z calculated for C₄₁H₆₃N₈O₉S [M+H]⁺ 843.44, found 843.38.

Intermediate 38d (50 mg, 0.05 mmol) dissolved in 5 mL DMF was slowly added to a solution of HATU (38 mg, 0.1 mmol) and DIPEA (0.05 mL, 0.25 mmol) in 5 mL DMF during 30 min. The reaction was stirred for another 30 min and then the solvent was evaporated. The remaining crude product was purified with preparative HPLC using the C18 reverse phase column (Waters, Sunfire™ Prep C18 OBD™, 5 μm, 50×100 mm) to yield 39d. White powder, MS (ESI): m/z calculated for C₄₁H₆₁N₈O₈S [M+H]⁺ 825.43, found 825.36.

The cyclic product 39d was then dissolved in trifluoroacetic acid:H₂O (95:5) and stirred at room temperature for 2h in order to remove the Pbf group from arginine guanidine. Then the solvent was evaporated and the crude product was purified with preparative HPLC using the C18 reverse phase column (Waters, Sunfire™ Prep C18 OBD™, 5 μm, 50×100 mm). The final compound Cpd. No. 73 then was dissolved in CH₃CN:H₂O (1:1) and lyophilized. White solid (12.7% yield over 4 steps). HRMS (ESI): m/z calculated for C₂₈H₄₅N₈O₅ [M+H]⁺ 573.3513, found 573.3507. ¹H NMR (400 MHz, CD₃OD) δ 7.50-7.28 (m, 5H), 5.14 (s, 1H), 4.28 (t, J=6.8 Hz, 1H), 4.13 (dd, J=10.4, 5.2 Hz, 1H), 4.08-3.99 (m, 1H), 3.54-3.41 (m, 1H), 3.18 (td, J=7.1, 2.5 Hz, 2H), 3.04-2.97 (m, 1H), 2.62-2.51 (m, 1H), 1.94-1.54 (m, 9H), 1.47-1.24 (m, 3H), 1.13 (dd, J=6.9, 3.3 Hz, 6H), 0.90 (t, J=7.5 Hz, 3H). ¹³C NMR (101 MHz, CD₃OD) δ 180.51, 175.63, 174.34, 173.64, 172.92, 158.63, 137.30, 129.92, 129.67, 129.31, 61.54, 57.09, 56.08, 55.83, 41.61, 39.68, 35.72, 32.14, 29.95, 29.24, 26.68, 26.53, 23.37, 19.93, 19.74, 10.25.

### Synthesis of N-((3R,6S,9S,12R)-6-ethyl-9-(3-guanidinopropyl)-12-methyl-2,5,8,11-tetraoxo-3-phenyl-1,4,7,10-tetraazacyclotetradecan-12-yl)isobutyramide (Cpd. No. 4) CF₃COOH salt

Fmoc-D-Phg-OH (0.5 mmol, 0.17 g) was loaded on the 0.1 mmol 2-chlorotrityl chloride (24) resin (ChemPep) (1 mmol/g) overnight in CH₂Cl₂ and in the presence of 2,4,6-collidine (3 mmol, 0.4 mL). Then, the resin was washed with DMF, MeOH, CH₂C₂, respectively, mixed with DIPEA (0.29 mmol, 0.5 mL) in MeOH:CH₂Cl₂ (1:5) and was shaken for 30 min to endcap unreacted 2-chlorotrityl group on the resin. Next, classical chain elongation was carried out with Fmoc chemistry. The peptide intermediate was cleaved from the resin by treatment of 41a with 4 ml of 1% trifluoroacetic acid in CH₂Cl₂ (3×10 min). The filtrate was evaporated and the residue was dissolved in anhydrous ethanol. Pd/C (20 mg) was added to the flask and the reaction was stirred at H₂ atmosphere for 12h at 50° C. Then the reaction was filtered, the filtrate was evaporated and the remaining crude product was purified with preparative HPLC using the C18 reverse phase column (Waters, Sunfire™ Prep C18 OBD™, 5 μm, 50×100 mm) to yield 42a. White powder, MS (ESI): m/z calculated for C₄₀H₆₁N₈O₉S [M+H]⁺ 829.43, found 829.25.

Then the method applied for Cpd. No. 73 starting from 38d was used to make Cpd. No. 4 starting from 42a. White solid (8.7% yield over 4 steps). HRMS (ESI): m/z calculated for C₂₇H₄₃N₈O₅ [M+H]⁺ 559.3352, found 559.3351. ¹H NMR (400 MHz, Methanol-d₄) δ 7.49-7.37 (m, 2H), 7.37-7.20 (m, 3H), 5.46 (d, J=9.2 Hz, 1H), 4.38 (dd, J=10.5, 3.8 Hz, 1H), 4.24-4.06 (m, 1H), 3.85-3.63 (m, 1H), 3.26-3.16 (m, 2H), 2.76-2.56 (m, 3H), 2.24-2.07 (m, 1H), 1.92-1.59 (m, 5H), 1.53 (s, 3H), 1.40-1.32 (m, 1H), 1.23 (d, J=6.9 Hz, 3H), 1.16 (d, J=6.7 Hz, 3H), 0.96 (t, J=7.4 Hz, 3H). ¹³C NMR (101 MHz, MeOD) δ 182.07, 177.62, 175.47, 172.28, 172.11, 158.66, 139.36, 129.48, 129.42, 128.86, 59.98, 58.21, 57.51, 55.53, 41.72, 38.63, 36.40, 35.98, 28.83, 26.67, 24.75, 21.17, 18.90, 11.06.

### Synthesis of N-((3R,6S,9S,12R)-6-ethyl-9-(3-guanidinopropyl)-12-methyl-2,5,8,11-tetraoxo-3-phenyl-1,4,7,10-tetraazacyclopentadecan-12-yl)isobutyramide (Cpd. No. 74) CF₃COOH salt

A method applied for Cpd. No. 73 was used to make Cpd. No. 74 starting from 24. White solid (19.4% yield over 4 steps). HRMS (ESI): m/z calculated for C₂₈H₄₅N₈O₅ [M+H]⁺ 573.3513, found 573.3506. ¹H NMR (400 MHz, CD₃OD) δ 7.55-7.23 (m, 5H), 5.35 (s, 1H), 4.40 (dd, J=10.0, 4.7 Hz, 1H), 4.16 (dd, J=8.6, 6.3 Hz, 1H), 3.41-3.33 (m, 1H), 3.24-3.12 (m, 3H), 2.65-2.37 (m, 1H), 2.00-1.48 (m, 10H), 1.46 (s, 3H), 1.12 (dd, J=6.8, 2.5 Hz, 6H), 0.94 (t, J=7.4 Hz, 3H). ¹³C NMR (101 MHz, CD₃OD) δ 179.91, 176.79, 174.82, 174.33, 172.75, 158.62, 138.00, 129.82, 129.37, 128.93, 60.47, 60.37, 56.91, 55.12, 41.74, 40.12, 37.57, 35.81, 28.76, 26.24, 25.14, 23.68, 21.04, 19.97, 19.36, 11.00.

### Synthesis of N-((3R,6S,9S,12R)-6-ethyl-9-(3-guanidinopropyl)-12-methyl-2,5,8,11-tetraoxo-3-phenyl-1,4,7,10-tetraazacycloheptadecan-12-yl)isobutyramide (Cpd. No. 75) CF₃COOH salt

Cpd. No. 75 was prepared according Method B starting from 36b. The RCM cyclization was achieved at room temperature overnight. White solid (28% yield over 3 steps). HRMS (ESI): m/z calc. for C₃₀H₄₉N₈O₅ [M+H]⁺ 601.3820, found 601.3827. ¹H NMR (300 MHz, CD₃OD) δ: 7.45-7.32 (m, 5H), 5.22 (s, 1H), 4.45-4.31 (m, 2H), 3.28-3.13 (m, 4H), 2.59-2.49 (m, 1H), 1.97-1.05 (m, 23H), 0.89 (t, J=7.5 Hz, 3H). ¹³C NMR (75 MHz, CD₃OD) δ: 179.80, 176.66, 174.54, 173.62, 173.32, 158.81, 137.54, 130.08, 129.79, 129.44, 61.04, 60.91, 55.74, 54.59, 41.83, 41.69, 39.95, 36.05, 30.64, 29.75, 27.18, 27.10, 26.14, 25.08, 21.12, 20.33, 19.48, 10.18.

### Synthesis of N-((3R,6S,9S,12R)-6-ethyl-9-(3-guanidinopropyl)-12-methyl-2,5,8,11-tetraoxo-3-phenyl-1,4,7,10-tetraazacyclooctadecan-12-yl)isobutyramide (Cpd. No. 76) CF₃COOH salt

Cpd. No. 76 was prepared according Method B starting from 36c. The RCM cyclization was achieved at room temperature overnight. White solid (46% yield over 3 steps). HRMS (ESI): m/z calc. for C₃₁H₅₁N₈O₅ [M+H]⁺ 615.3977, found 615.3976. ¹H NMR (300 MHz, CD₃OD) δ: 7.45-7.29 (m, 5H), 5.40 (s, 1H), 4.29-4.19 (m, 2H), 3.59-3.47 (m, 1H), 3.19 (t, J=6.7 Hz, 2H), 3.09-2.96 (m, 1H), 2.63-2.52 (m, 1H), 2.02-1.59 (m, 8H), 1.58-1.27 (m, 11H), 1.14 (d, J=6.5 Hz, 3H), 1.12 (d, J=6.5 Hz, 3H), 0.89 (t, J=7.4 Hz, 3H). ¹³C NMR (75 MHz, CD₃OD) δ: 180.53, 177.59, 174.48, 173.99, 172.88, 158.86, 138.38, 129.97, 129.57, 60.67, 59.87, 56.51, 55.22, 42.04, 39.80, 37.53, 36.09, 29.98, 28.42, 28.38, 26.42, 25.37, 22.72, 22.47, 20.08, 19.94, 10.69.

### Synthesis of N-((3R,6S,9S,12R)-6-ethyl-9-(3-guanidinopropyl)-12-methyl-2,5,8,11-tetraoxo-3-phenyl-1,4,7,10-tetraazacycloicosan-12-yl)isobutyramide (Cpd. No. 77) CF₃COOH salt

Cpd. No. 77 was prepared according Method B starting from 36d. The RCM cyclization was achieved at room temperature overnight. White solid (27% yield over 3 steps). HRMS (ESI): m/z calc. for C₃₃H₅₅N₈O₅ [M+H]⁺ 643.4290, found 643.4292. ¹H NMR (300 MHz, CD₃OD) δ: 7.41-7.30 (m, 5H), 5.40 (s, 1H), 4.34-4.28 (m, 1H), 4.18 (t, J=7.1 Hz, 1H), 3.55-3.44 (m, 1H), 3.17 (t, J=6.9 Hz, 2H), 3.07-2.95 (m, 1H), 2.61-2.50 (m, 1H), 2.00-1.28 (m, 23H), 1.16-1.07 (m, 6H), 0.92 (t, J=7.4 Hz, 3H). ¹³C NMR (75 MHz, CD₃OD) δ: 180.22, 176.92, 174.53, 174.30, 172.69, 158.81, 138.70, 130.03, 129.55, 129.39, 60.72, 59.51, 57.11, 54.55, 42.14, 40.03, 38.56, 36.13, 30.28, 29.31, 28.92, 28.63, 28.04, 26.56, 26.16, 26.10, 23.50, 22.76, 20.05, 10.81.

### Synthesis of N-((3R,6S,9S,12R)-6-ethyl-12-methyl-9-(3-(3-methylguanidino)propyl)-2,5,8,11-tetraoxo-3-phenyl-1,4,7,10-tetraazacyclotetradecan-12-yl)isobutyramide (Cpd. No. 5) CF₃COOH salt

A method applied for Cpd. No. 4 was used to make Cpd. No. 5 starting from intermediate 24. White solid (15.4% yield over 4 steps). HRMS (ESI): m/z calculated for C₂₈H₄₅N₈O₅ [M+H]⁺ 573.3507, found 573.3511. ¹H NMR (400 MHz, Methanol-d₄) δ 7.44-7.37 (m, 2H), 7.37-7.26 (m, 3H), 5.48-5.44 (m, 1H), 4.38 (dd, J=10.5, 3.7 Hz, 1H), 4.17 (td, J=7.0, 3.2 Hz, 1H), 3.80-3.72 (m, 1H), 3.28-3.16 (m, 2H), 2.83 (s, 3H), 2.75-2.57 (m, 3H), 2.25-2.11 (m, 1H), 1.96-1.69 (m, 4H), 1.53 (s, 3H), 1.41-1.32 (m, 1H), 1.22 (d, J=6.9 Hz, 3H), 1.16 (d, J=6.7 Hz, 3H), 0.95 (t, J=7.4 Hz, 3H). ¹³C NMR (101 MHz, MeOD) δ 182.17, 177.63, 175.52, 172.28, 172.10, 158.21, 139.37, 129.49, 129.41, 128.86, 60.07, 58.20, 57.47, 55.52, 41.75, 38.63, 36.41, 36.02, 28.88, 28.31, 26.64, 24.75, 21.18, 18.90, 11.06.

Cpd. Nos. 101-105 were synthesized using the procedure used in the synthesis of Cpd. No. 4.

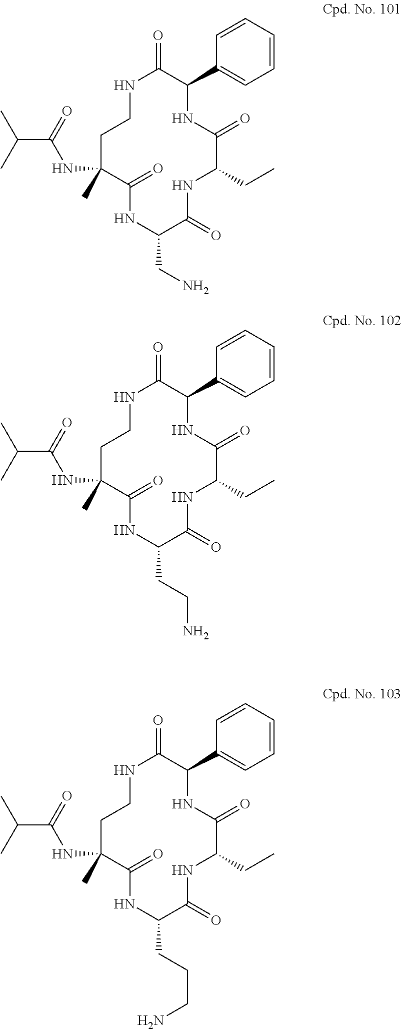

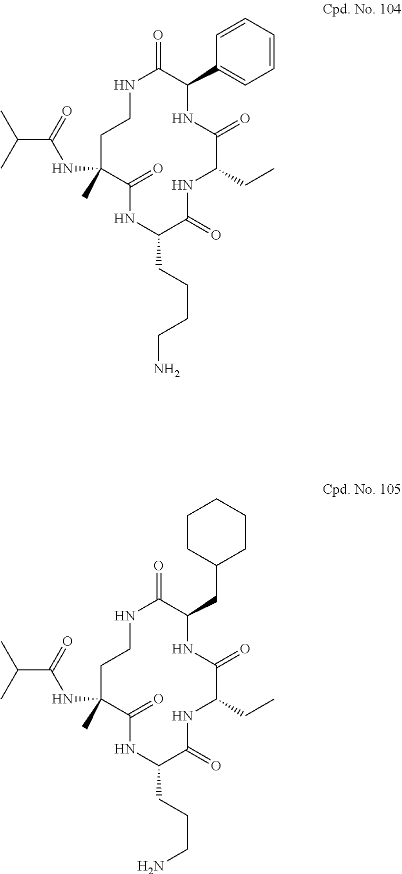

### Synthesis of N-((3S,6S,9S,12R)-6-ethyl-12-methyl-9-(3-(3-methylguanidino)propyl)-2,5,8,11-tetraoxo-3-phenyl-1,4,7,10-tetraazacyclotetradecan-12-yl)isobutyramide (Cpd. No. 62) CF₃COOH salt

Cpd. No. 62 was obtained in the synthesis of starting from Cpd. No. 5 from 24, as D-α-phenylglycine was isomerized to L-α-phenylglycine during the solid phase peptide synthesis. White solid (7% yield over 4 steps). HRMS (ESI): m/z calculated for C₂₈H₄₅N₈O₅ [M+H]⁺ 573.3507, found 573.3504. ¹H NMR (300 MHz, Methanol-d₄) δ 7.36-7.19 (m, 5H), 5.21 (s, 1H), 4.27 (dd, J=8.8, 6.3 Hz, 1H), 4.00 (dd, J=8.5, 6.0 Hz, 1H), 3.38 (dt, J=14.6, 3.9 Hz, 1H), 3.28-3.14 (m, 2H), 3.04 (t, J=13.5 Hz, 1H), 2.84 (s, 3H), 2.70-2.53 (m, 2H), 2.00-1.60 (m, 7H), 1.55 (s, 3H), 1.20 (d, J=5.5 Hz, 3H), 1.18 (d, J=5.3 Hz, 3H), 0.91 (t, J=7.4 Hz, 3H). ¹³C NMR (101 MHz, MeOD) δ 181.09, 177.37, 174.97, 173.54, 171.64, 158.20, 138.80, 129.24, 128.74, 128.60, 60.17, 59.82, 57.73, 57.65, 41.83, 37.48, 37.15, 36.12, 28.51, 28.30, 26.74, 26.11, 21.12, 19.18, 10.88.

### Synthesis of N-((3R,6S,9S,12R)-9-(4-(dimethylamino)butyl)-6-ethyl-12-methyl-2,5,8,11-tetraoxo-3-phenyl-1,4,7,10-tetraazacyclotetradecan-12-yl)isobutyramide (Cpd. No. 6) CF₃COOH salt

Cpd. No. 6 was obtained using the procedure applied for Cpd. No. 9 from Cpd. No. 104 in 32% yield. White solid. >98% purity. MS (ESI): m/z calculated for C₂₉H₄₇N₆O₅ [M+H]⁺ 559.36, found 559.45. ¹H NMR (400 MHz, Methanol-d₄) δ 8.60 (s, 1H), 8.32 (d, J=4.1 Hz, 1H), 8.18 (d, J=9.5 Hz, 1H), 8.00 (d, J=9.2 Hz, 1H), 7.87 (d, J=9.3 Hz, 1H), 7.58-7.39 (m, 2H), 7.39-7.16 (m, 3H), 5.46 (d, J=9.2 Hz, 1H), 4.37 (td, J=10.1, 3.8 Hz, 1H), 4.14 (td, J=7.5, 7.0, 4.0 Hz, 1H), 3.82-3.69 (m, 1H), 3.12 (dd, J=9.2, 6.9 Hz, 2H), 2.88 (s, 6H), 2.75-2.57 (m, 3H), 2.17 (dtd, J=14.8, 7.4, 3.9 Hz, 1H), 1.95-1.83 (m, 2H), 1.83-1.67 (m, 3H), 1.61-1.41 (m, 5H), 1.40-1.32 (m, 1H), 1.23 (d, J=6.9 Hz, 3H), 1.16 (d, J=6.7 Hz, 3H), 0.95 (t, J=7.4 Hz, 3H).

### Synthesis of N-((3R,6S,12R)-6-ethyl-12-methyl-2,5,8,11-tetraoxo-9-phenethyl-3-phenyl-1,4,7,10-tetraazacyclotetradecan-12-yl)isobutyramide (Cpd. No. 13) CF₃COOH salt

Cpd. No. 13 was synthesized using the procedure applied for Cpd. No. 4 to yield 3.9 mg, white powder, 7% yield over 3 steps. >98% purity. MS (ESI): m/z calculated for C₃₁H₄₂N₅O₅ [M+H]⁺ 564.32, found 564.39. ¹H NMR (400 MHz, Methanol-d₄) δ 8.56 (s, 1H), 8.31 (d, J=4.3 Hz, 1H), 8.21 (d, J=9.5 Hz, 1H), 7.99 (d, J=9.2 Hz, 1H), 7.88 (d, J=9.2 Hz, 1H), 7.53-7.38 (m, 2H), 7.38-7.25 (m, 5H), 7.25-7.08 (m, 3H), 5.46 (d, J=9.2 Hz, 1H), 4.39 (td, J=10.1, 3.8 Hz, 1H), 4.10 (ddd, J=8.1, 5.6, 4.2 Hz, 1H), 3.84-3.68 (m, 1H), 2.86-2.56 (m, 5H), 2.24-2.01 (m, 3H), 1.86-1.71 (m, 1H), 1.55 (s, 3H), 1.42-1.32 (m, 1H), 1.23 (d, J=6.9 Hz, 3H), 1.17 (d, J=6.6 Hz, 3H), 0.97 (t, J=7.4 Hz, 3H).

### Synthesis of N-((3R,6S,12R)-6-ethyl-12-methyl-2,5,8,11-tetraoxo-3-phenyl-9-(3-phenylpropyl)-1,4,7,10-tetraazacyclotetradecan-12-yl)isobutyramide (Cpd. No. 14) CF₃COOH salt

Cpd. No. 14 was synthesized using the procedure applied for Cpd. No. 4 to yield 3.9 mg, white powder, 7% yield over 3 steps. >98% purity. MS (ESI): m/z calculated for C₃₂H₄₄N₅O₅ [M+H]⁺ 578.33, found 578.43. ¹H NMR (400 MHz, Methanol-d₄) δ 8.53 (s, 1H), 8.20 (d, J=3.9 Hz, 1H), 8.11 (d, J=9.5 Hz, 1H), 8.02 (d, J=9.2 Hz, 1H), 7.87 (d, J=9.4 Hz, 1H), 7.46-7.37 (m, 2H), 7.37-7.21 (m, 5H), 7.21-7.06 (m, 3H), 5.45 (d, J=9.2 Hz, 1H), 4.36 (td, J=10.1, 3.7 Hz, 1H), 4.19-4.05 (m, 1H), 3.84-3.68 (m, 1H), 2.76-2.49 (m, 5H), 2.16 (ddd, J=14.2, 7.4, 3.8 Hz, 1H), 1.90-1.67 (m, 5H), 1.51 (s, 3H), 1.37-1.32 (m, 1H), 1.20 (d, J=6.9 Hz, 3H), 1.12 (d, J=6.7 Hz, 3H), 0.91 (t, J=7.4 Hz, 3H).

### Synthesis of N-((3R,6S,12R)-9-(3-amino-3-oxopropyl)-6-ethyl-12-methyl-2,5,8,11-tetraoxo-3-phenyl-1,4,7,10-tetraazacyclotetradecan-12-yl)isobutyramide (Cpd. No. 15) CF₃COOH salt

Cpd. No. 15 was synthesized using the procedure applied for Cpd. No. 4 to yield 8.8 mg, white powder, 17% yield over 3 steps. >98% purity. MS (ESI): m/z calculated for C₂₆H₃₉N₆O₆ [M+H]⁺ 531.29, found 531.36.

### Synthesis of N-((3R,6S,9S,12R)-6-ethyl-12-methyl-2,5,8,11-tetraoxo-3-phenyl-9-(3-ureidopropyl)-1,4,7,10-tetraazacyclotetradecan-12-yl)isobutyramide (Cpd. No. 16) CF₃COOH salt

Cpd. No. 16 was synthesized using the procedure applied for Cpd. No. 4 to yield 3.2 mg, white powder, 6% yield over 3 steps. >98% purity. MS (ESI): m/z calculated for C₂₇H₄₂N₇O₆ [M+H]⁺ 560.32, found 560.26. ¹H NMR (400 MHz, Methanol-d₄) δ 8.57 (s, 1H), 8.48 (s, 1H), 8.18 (d, J=9.5 Hz, 1H), 8.04 (d, J=9.2 Hz, 1H), 7.52-7.40 (m, 2H), 7.40-7.15 (m, 4H), 5.46 (d, J=9.3 Hz, 1H), 4.39 (td, J=10.1, 3.7 Hz, 1H), 4.14 (td, J=6.5, 3.6 Hz, 1H), 3.83-3.71 (m, 1H), 3.17 (q, J=6.6 Hz, 2H), 2.77-2.60 (m, 3H), 2.19 (ddt, J=14.9, 11.3, 7.2 Hz, 1H), 1.93-1.77 (m, 3H), 1.67-1.59 (m, 2H), 1.54 (s, 3H), 1.39-1.33 (m, 1H), 1.22 (d, J=6.9 Hz, 3H), 1.16 (d, J=6.7 Hz, 3H), 0.96 (t, J=7.4 Hz, 3H).

The following Compounds of the Disclosure were prepared using the synthetic methods described above.

N-((3R,6S,9S,12R)-6-ethyl-12-methyl-9-(((1-methylpiperidin-4-yl)amino)methyl)-2,5,8,11-tetraoxo-3-phenyl-1,4,7,10-tetraazacyclotetradecan-12-yl)isobutyramide (Cpd. No. 7) CF₃COOH salt. Cpd. No. 7 was synthesized using the procedure applied for Cpd. No. 9 from Cpd. No. 101 to yield 3.8 mg, white powder, 65% yield. >98% purity. MS (ESI): m/z calculated for C₃₀H₄₈N₇O₅[M+H]⁺ 586.37, found 586.40.

N-((3R,6S,9S,12R)-6-ethyl-12-methyl-9-(2-((1-methylpiperidin-4-yl)amino)ethyl)-2,5,8,11-tetraoxo-3-phenyl-1,4,7,10-tetraazacyclotetradecan-12-yl)isobutyramide (Cpd. No. 8) CF₃COOH salt. Cpd. No. 8 was synthesized using the procedure applied for Cpd. No. 9 from Cpd. No. 172 to yield 3.8 mg, white powder, 65% yield. >98% purity. MS (ESI): m/z calculated for C₃₁H₅₀N₇O₅[M+H]⁺ 600.39, found 600.47. ¹H NMR (400 MHz, Methanol-d₄) δ 8.64 (s, 1H), 7.96 (d, J=9.2 Hz, 1H), 7.94-7.83 (m, 1H), 7.47-7.38 (m, 2H), 7.38-7.21 (m, 3H), 5.47 (d, J=9.2 Hz, 1H), 4.40 (dd, J=10.5, 3.9 Hz, 1H), 4.30 (t, J=7.5 Hz, 1H), 3.77 (t, J=10.1 Hz, 1H), 3.64 (d, J=12.5 Hz, 2H), 3.53-3.40 (m, 1H), 3.39-3.30 (m, 1H), 3.29-3.21 (m, 1H), 3.20-3.00 (m, 2H), 2.89 (s, 3H), 2.77-2.56 (m, 3H), 2.49-2.32 (m, 2H), 2.32-2.08 (m, 3H), 2.08-1.87 (m, 2H), 1.81 (ddd, J=14.2, 10.5, 7.3 Hz, 1H), 1.54 (s, 3H), 1.40-1.31 (m, 1H), 1.22 (d, J=6.9 Hz, 3H), 1.15 (d, J=6.6 Hz, 3H), 0.95 (t, J=7.4 Hz, 3H).

N-((3R,6S,9S,12R)-6-ethyl-12-methyl-9-(3-((1-methylpiperidin-4-yl)amino)propyl)-2,5,8,11-tetraoxo-3-phenyl-1,4,7,10-tetraazacyclotetradecan-12-yl)isobutyramide (Cpd. No. 9) CF₃COOH salt.

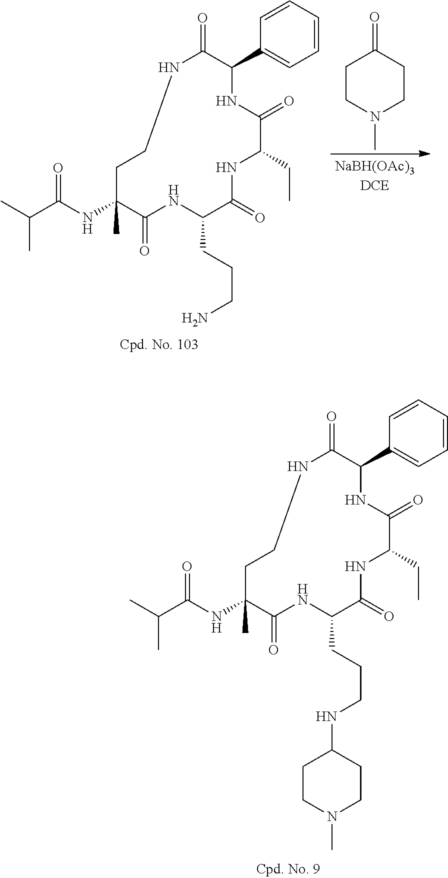

Cpd. No. 103 (18 mg, 0.035 mmol) was dissolved in DCE (2 mL). N-methyl-4-piperidone (6.5 uL, 0.5 mmol) and NaBH(OAc)₃ (30 mg, 0.14 mmol) were added to the reaction. The reaction was stirred at room temperature for 4 hr and then quenched with saturated ammonium chloride solution (10 mL). The product was extracted with ethyl acetate (3*10 mL) and the combined organic solution was dried and concentrated under vacuum. The residue was purified with preparative HPLC using the C18 reverse phase column (Waters, Sunfire™ Prep Cis OBD™, 5 μm, 50×100 mm), yielding Cpd. No. 9 (10.7 mg, white powder, 50% yield). >98% purity. MS (ESI): m/z calculated for C₃₂H₅₂N₇O₅[M+H]⁺ 614.40, found 614.53. ¹H NMR (400 MHz, Methanol-d₄) δ 8.61 (s, 1H), 8.43 (d, J=4.1 Hz, 1H), 8.21 (d, J=9.5 Hz, 1H), 7.99 (d, J=9.1 Hz, 1H), 7.89 (dd, J=9.4, 2.6 Hz, 1H), 7.45-7.37 (m, 2H), 7.37-7.24 (m, 3H), 5.46 (d, J=9.2 Hz, 1H), 4.38 (td, J=10.1, 3.8 Hz, 1H), 4.15 (dd, J=7.6, 4.1 Hz, 1H), 3.79-3.71 (m, 1H), 3.65 (d, J=12.6 Hz, 2H), 3.48-3.38 (m, 1H), 3.17-3.08 (m, 3H), 2.88 (s, 3H), 2.74-2.61 (m, 3H), 2.38 (d, J=13.5 Hz, 2H), 2.18 (ddd, J=14.2, 7.4, 3.9 Hz, 1H), 1.95-1.75 (m, 6H), 1.53 (s, 3H), 1.43-1.24 (m, 2H), 1.22 (d, J=6.9 Hz, 3H), 1.15 (d, J=6.6 Hz, 3H), 0.95 (t, J=7.4 Hz, 3H).

N-((3R,6S,9S,12R)-6-ethyl-12-methyl-2,5,8,11-tetraoxo-3-phenyl-9-((pyridin-2-ylamino)methyl)-1,4,7,10-tetraazacyclotetradecan-12-yl)isobutyramide (Cpd. No. 10) CF₃COOH salt. Cpd. No. 10 was synthesized using the procedure applied for Cpd. No. 12 from Cpd. No. 101 to yield 3.1 mg, white powder, 54% yield. LC-MS(ESI) m/z (M+H)⁺: 566.32; calcd for C₂₉H₄₀N₇O₅ (M+H)⁺: 566.31; >98% purity.

N-((3R,6S,12R)-6-ethyl-12-methyl-2,5,8,11-tetraoxo-3-phenyl-9-(2-(pyridin-2-ylamino)ethyl)-1,4,7,10-tetraazacyclotetradecan-12-yl)isobutyramide (Cpd. No. 11) CF₃COOH salt. Cpd. No. 11 was synthesized using the procedure applied for Cpd. No. 12 from Cpd. No. 102 to yield 4.4 mg, white powder, 54% yield. >98% purity. MS (ESI): m/z calculated for C₃₀H₄₂N₇O₅[M+H]⁺ 580.32, found 580.27.

N-((3R,6S,9S,12R)-6-ethyl-12-methyl-2,5,8,11-tetraoxo-3-phenyl-9-(3-(pyridin-2-ylamino)propyl)-1,4,7,10-tetraazacyclotetradecan-12-yl)isobutyramide (Cpd. No. 12) CF₃COOH salt.

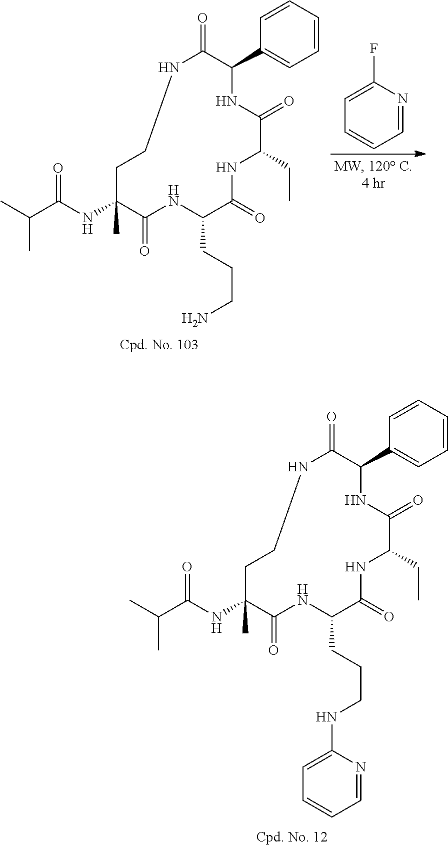 [SMILES: CC[C@@H]1NC(=O)[C@H](CCCN)NC(=O)[C@](C)(NC(=O)C(C)C)CCNC(=O)[C@@H](c2ccccc2)NC1=O ; Fc1ccccn1 ; CC[C@@H]1NC(=O)[C@H](CCCNc2ccccn2)NC(=O)[C@](C)(NC(=O)C(C)C)CCNC(=O)[C@@H](c2ccccc2)NC1=O]

Cpd. No. 103 (5 mg, 0.01 mmol) was dissolved in 2-fluoropyridine (0.2 mL). The reaction was heated to 120° C. for 4 hr under microwave. Then the reaction was cooled to room temperature and the residue was purified with preparative HPLC using the C18 reverse phase column (Waters, Sunfire™ Prep Cis OBD™, 5 μm, 50×100 mm), yielding Cpd. No. 12 (2.6 mg, white powder, 45% yield). >98% purity. MS (ESI): m/z calculated for C₃₁H₄₄N₇O₅[M+H]⁺ 594.34, found 594.34. ¹H NMR (400 MHz, Methanol-d₄) δ 8.61 (s, 1H), 8.37 (d, J=4.0 Hz, 1H), 8.23 (d, J=9.5 Hz, 1H), 7.99 (d, J=9.2 Hz, 1H), 7.95-7.78 (m, 3H), 7.47-7.38 (m, 2H), 7.37-7.23 (m, 3H), 7.05 (d, J=9.1 Hz, 1H), 6.89 (t, J=6.7 Hz, 1H), 5.47 (d, J=9.2 Hz, 1H), 4.39 (td, J=10.0, 3.7 Hz, 1H), 4.27-4.15 (m, 1H), 3.76 (t, J=11.2 Hz, 1H), 3.52-3.38 (m, 2H), 2.78-2.57 (m, 3H), 2.18 (ddd, J=14.2, 7.5, 4.0 Hz, 1H), 1.99-1.72 (m, 5H), 1.53 (s, 3H), 1.40-1.29 (m, 2H), 1.22 (d, J=6.8 Hz, 3H), 1.16 (d, J=6.5 Hz, 3H), 0.93 (t, J=7.4 Hz, 3H)

N-((3R,6S,9S,12R)-9-(3-((4,5-dihydro-1H-imidazol-2-yl)amino)propyl)-6-ethyl-12-methyl-2,5,8,11-tetraoxo-3-phenyl-1,4,7,10-tetraazacyclotetradecan-12-yl)isobutyramide (Cpd. No. 17) CF₃COOH salt.

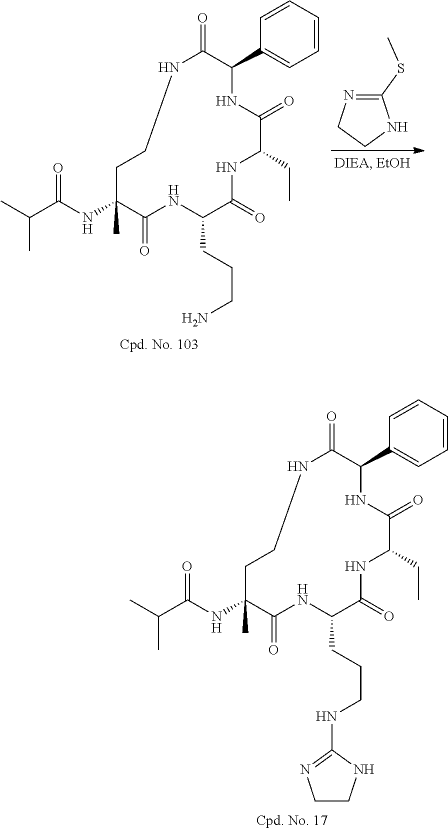

Cpd. No. 103 (5 mg, 0.01 mmol) was dissolved in Ethanol (0.2 mL) and DIEA (0.05 mL). 2-Methylthio-2-imidazoline hydroiodide (25 mg, 0.1 mmol) was added to the reaction. The reaction was heated to 120° C. for 4 hr under microwave under there is no starting materials left checked by UPLC. Then the reaction was cooled to room temperature and the residue was purified with preparative HPLC using the C18 reverse phase column (Waters, Sunfire™ Prep C18 OBD™, 5 μm, 50×100 mm), yielding Cpd. No. 17 (3.1 mg, white powder, 53% yield). >98% purity. MS (ESI): m/z calculated for C₂₉H₄₅N₈O₅[M+H]⁺ 585.35, found 585.47. ¹H NMR (400 MHz, Methanol-d₄) δ 8.60 (s, 1H), 8.33 (d, J=4.1 Hz, 1H), 8.20 (d, J=9.6 Hz, 1H), 7.99 (d, J=9.2 Hz, 1H), 7.87 (d, J=9.3 Hz, 1H), 7.44-7.38 (m, 2H), 7.36-7.25 (m, 3H), 5.46 (d, J=9.2 Hz, 1H), 4.38 (td, J=10.1, 3.8 Hz, 1H), 4.15 (td, J=7.0, 4.1 Hz, 1H), 3.81-3.73 (m, 1H), 3.70 (s, 4H), 3.29-3.22 (m, 2H), 2.76-2.62 (m, 3H), 2.18 (ddt, J=15.0, 7.5, 3.7 Hz, 1H), 1.92-1.71 (m, 5H), 1.53 (s, 3H), 1.40-1.30 (m, 2H), 1.23 (d, J=6.9 Hz, 3H), 1.16 (d, J=6.6 Hz, 3H), 0.95 (t, J=7.4 Hz, 3H).

N-((3R,6S,9S,12R)-9-(2-((4,5-dihydro-1H-imidazol-2-yl)amino)ethyl)-6-ethyl-12-methyl-2,5,8,11-tetraoxo-3-phenyl-1,4,7,10-tetraazacyclotetradecan-12-yl)isobutyramide (Cpd. No. 18) CF₃COOH salt. Cpd. No. 18 was synthesized using the procedure applied for Cpd. No. 17 from Cpd. No. 102 to yield 2.7 mg, white powder, 47% yield. >98% purity. MS (ESI): m/z calculated for C₂₈H₄₃N₈O₅[M+H]⁺ 571.34, found 571.51. ¹H NMR (400 MHz, Methanol-d₄) δ 8.62 (s, 1H), 8.46 (d, J=4.5 Hz, 1H), 8.29 (d, J=9.5 Hz, 1H), 7.95 (d, J=9.1 Hz, 1H), 7.85 (d, J=9.0 Hz, 1H), 7.49-7.38 (m, 2H), 7.38-7.22 (m, 3H), 5.46 (d, J=9.1 Hz, 1H), 4.38 (td, J=10.0, 3.9 Hz, 1H), 4.26-4.16 (m, 1H), 3.84-3.66 (m, 5H), 3.50-3.36 (m, 2H), 2.74-2.60 (m, 3H), 2.18 (ddd, J=14.1, 7.4, 4.1 Hz, 1H), 2.08-1.99 (m, 2H), 1.80 (ddd, J=14.2, 10.5, 7.2 Hz, 1H), 1.53 (s, 3H), 1.43-1.31 (m, 2H), 1.23 (d, J=6.9 Hz, 3H), 1.16 (d, J=6.7 Hz, 3H), 0.95 (t, J=7.4 Hz, 3H).

N-((3R,6S,9S,12R)-9-(2-((1H-imidazol-2-yl)amino)ethyl)-6-ethyl-12-methyl-2,5,8,11-tetraoxo-3-phenyl-1,4,7,10-tetraazacyclotetradecan-12-yl)isobutyramide (Cpd. No. 19) CF₃COOH salt. Cpd. No. 19 was synthesized using the procedure applied for Cpd. No. 17 from Cpd. No. 102 to yield 0.6 mg, white powder, 10% yield. >98% purity. MS (ESI): m/z calculated for C₂₈H₄₁N₈O₅[M+H]⁺ 569.32, found 569.27.

N-((3R,6S,9S,12R)-9-(3-((1H-imidazol-2-yl)amino)propyl)-6-ethyl-12-methyl-2,5,8,11-tetraoxo-3-phenyl-1,4,7,10-tetraazacyclotetradecan-12-yl)isobutyramide (Cpd. No. 20) CF₃COOH salt. Cpd. No. 20 was synthesized using the procedure applied for Cpd. No. 17 from Cpd. No. 102 to yield 1.4 mg, white powder, 24% yield. >98% purity. MS (ESI): m/z calculated for C₂₉H₄₃N₈O₅[M+H]⁺ 583.34, found 583.31. ¹H NMR (400 MHz, Methanol-d₄) δ 8.80 (d, J=6.7 Hz, 1H), 8.63 (d, J=10.6 Hz, 1H), 8.33 (d, J=4.0 Hz, 1H), 8.19 (d, J=9.4 Hz, 1H), 7.99 (d, J=9.1 Hz, 1H), 7.86 (d, J=9.1 Hz, 1H), 7.52-7.23 (m, 5H), 6.89-6.82 (m, 1H), 5.45 (s, 1H), 4.38 (td, J=10.0, 3.9 Hz, 1H), 4.22-4.12 (m, 1H), 3.96-3.88 (m, 1H), 3.80-3.56 (m, 2H), 2.76-2.51 (m, 3H), 2.23-2.14 (m, 1H), 1.99-1.67 (m, 5H), 1.53 (s, 3H), 1.40-1.31 (m, 2H), 1.22 (d, J=6.9 Hz, 3H), 1.15 (d, J=6.6 Hz, 3H), 0.93 (t, J=7.4 Hz, 3H).

N-((3R,6S,12R)-6-ethyl-12-methyl-2,5,8,11-tetraoxo-3-phenyl-9-(3-(pyrimidin-2-ylamino)propyl)-1,4,7,10-tetraazacyclotetradecan-12-yl)isobutyramide (Cpd. No. 21) CF₃COOH salt. Cpd. No. 21 was synthesized using the procedure applied for Cpd. No. 17 from Cpd. No. 103 to yield 2.3 mg, white powder, 69% yield. >98% purity. MS (ESI): m/z calculated for C₃₀H₄₃N₈O₅[M+H]⁺ 595.34, found 585.43. ¹H NMR (400 MHz, Methanol-d₄) δ 8.60 (s, 1H), 8.42 (d, J=5.1 Hz, 2H), 8.31 (d, J=4.0 Hz, 1H), 8.17 (d, J=9.6 Hz, 1H), 8.02 (d, J=9.2 Hz, 1H), 7.87 (d, J=9.3 Hz, 1H), 7.50-7.22 (m, 5H), 6.79 (t, J=5.1 Hz, 1H), 5.46 (d, J=9.2 Hz, 1H), 4.38 (td, J=10.1, 3.7 Hz, 1H), 4.23-4.13 (m, 1H), 3.82-3.71 (m, 1H), 3.48 (td, J=7.0, 2.7 Hz, 2H), 2.75-2.56 (m, 3H), 2.22-2.11 (m, 1H), 1.96-1.86 (m, 2H), 1.86-1.71 (m, 3H), 1.53 (s, 3H), 1.36-1.28 (m, 2H), 1.21 (d, J=6.9 Hz, 3H), 1.12 (d, J=6.7 Hz, 3H), 0.93 (t, J=7.4 Hz, 3H).

N-((3R,6S,9S,12R)-6-ethyl-12-methyl-2,5,8,11-tetraoxo-3-phenyl-9-(3-((E)-2-(2,2,2-trifluoroethyl)guanidino)propyl)-1,4,7,10-tetraazacyclotetradecan-12-yl)isobutyramide (Cpd. No. 22) CF₃COOH salt. Cpd. No. 22 was synthesized using the procedure applied for Cpd. No. 4 to yield 1.1 mg, white powder, 2% yield over 4 steps. >98% purity. MS (ESI): m/z calculated for C₂₉H₄₄N₈O₅F₃ [M+H]⁺ 641.34, found 641.50.

N-(3-((5S,8R,13R)-5-ethyl-13-isobutyramido-13-methyl-3,6,9,14-tetraoxo-8-phenyl-1,4,7,10-tetraazacyclotetradecan-2-yl)propyl)acrylamide (Cpd. No. 23) CF₃COOH salt.

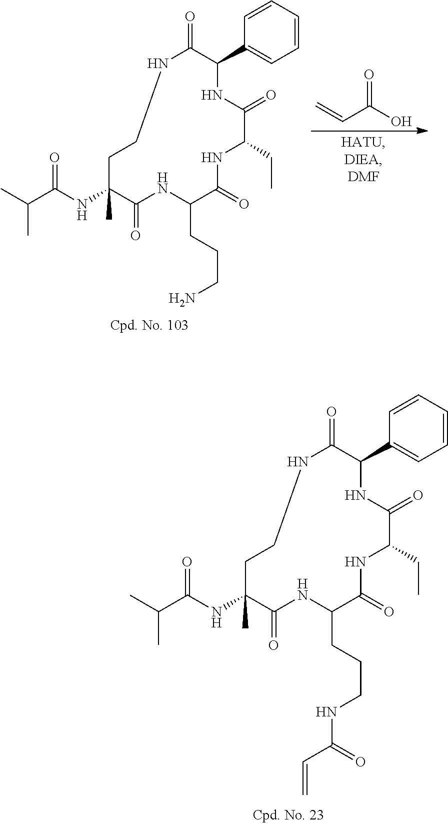

Cpd. No. 103 (4 mg, 0.008 mmol) and acrylic acid (0.8 uL, 0.012 mmol) were dissolved in DMF (0.5 mL). DIEA (7 uL, 0.04 mmol) and HATU (6 mg, 0.016 mmol) were added to the reaction. The reaction was stirred at room temperature for 2 hr. The solvent was removed, and the residue was purified with preparative HPLC using the C18 reverse phase column (Waters, Sunfire™ Prep C18 OBD™, 5 μm, 50×100 mm), yielding Cpd. No. 23 (2.9 mg, white powder, 66% yield). >98% purity. MS (ESI): m/z calculated for C₂₉H₄₃N₆O₆[M+H]⁺ 571.32, found 571.36.

N-(4-((2S,5S,8R,13R)-5-ethyl-13-isobutyramido-13-methyl-3,6,9,14-tetraoxo-8-phenyl-1,4,7,10-tetraazacyclotetradecan-2-yl)butyl)acrylamide (Cpd. No. 24) CF₃COOH salt. Cpd. No. 24 was synthesized using the procedure applied for Cpd. No. 23 from Cpd. No. 104. (3.4 mg, white powder, 51% yield). >98% purity. MS (ESI): m/z calculated for C₃₀H₄₅N₆O₆[M+H]⁺ 585.34, found 585.39.

N-((3R,6S,12R)-9-(3-(2-chloroacetamido)propyl)-6-ethyl-12-methyl-2,5,8,11-tetraoxo-3-phenyl-1,4,7,10-tetraazacyclotetradecan-12-yl)isobutyramide (Cpd. No. 25) CF₃COOH salt. Cpd. No. 25 was synthesized using the procedure applied for Cpd. No. 23 with 2-Chloroacetric acid instead of acrylic acid from Cpd. No. 103. (3.6 mg, white powder, 78% yield). >98% purity. MS (ESI): m/z calculated for C₂₈H₄₂N₆O₆C₁[M+H]⁺ 593.29, found 593.31.

N-((3R,6S,9S,12R)-9-(3-(3-acetylguanidino)propyl)-3-(cyclohexylmethyl)-6-ethyl-12-methyl-2,5,8,11-tetraoxo-1,4,7,10-tetraazacyclotetradecan-12-yl)isobutyramide (Cpd. No. 26) CF₃COOH salt. Cpd. No. 26 was synthesized using the procedure applied for Cpd. No. 17 from Cpd. No. 105 to yield 2.1 mg, white powder, 36% yield. >98% purity. MS (ESI): m/z calculated for C₃₀H₅₃N₈O₆ [M+H]⁺ 621.41, found 621.53.

N-((3R,6S,9S,12R)-3-(cyclohexylmethyl)-9-(3-((4,5-dihydro-1H-imidazol-2-yl)amino)propyl)-6-ethyl-12-methyl-2,5,8,11-tetraoxo-1,4,7,10-tetraazacyclotetradecan-12-yl)isobutyramide (Cpd. No. 30) CF₃COOH salt. Cpd. No. 30 was synthesized using the procedure applied for Cpd. No. 17 from Cpd. No. 105 to yield 4.3 mg, white powder, 76% yield. >98% purity. MS (ESI): m/z calculated for C₃₀H₅₃N₈O₅ [M+H]⁺ 605.41, found 605.42.

N-((3R,6S,9S,12R)-3-(cyclohexylmethyl)-9-(3-((E)-2,3-dimethylguanidino)propyl)-6-ethyl-12-methyl-2,5,8,11-tetraoxo-1,4,7,10-tetraazacyclotetradecan-12-yl)isobutyramide (Cpd. No. 31) CF₃COOH salt. Cpd. No. 31 was synthesized using the procedure applied for Cpd. No. 17 from Cpd. No. 105 to yield 1.7 mg, white powder, 30% yield. >98% purity. MS (ESI): m/z calculated for C₃H₅₅N₈O₅ [M+H]⁺ 607.43, found 607.55.

N-((3R,6S,9S,12R)-3-(cyclohexylmethyl)-6-ethyl-12-methyl-9-(3-(3-nitroguanidino)propyl)-2,5,8,11-tetraoxo-1,4,7,10-tetraazacyclotetradecan-12-yl)isobutyramide (Cpd. No. 32) CF₃COOH salt. Cpd. No. 32 was synthesized using the procedure applied for Cpd. No. 17 from Cpd. No. 105 to yield 5.4 mg, white powder, 93% yield. >98% purity. MS (ESI): m/z calculated for C₂₈H₅ON₉O₇ [M+H]⁺ 624.38, found 624.36.

N-((3R,6S,9S,12R)-9-(3-(3-cyanoguanidino)propyl)-3-(cyclohexylmethyl)-6-ethyl-12-methyl-2,5,8,11-tetraoxo-1,4,7,10-tetraazacyclotetradecan-12-yl)isobutyramide (Cpd. No. 33) CF₃COOH salt. Cpd. No. 33 was synthesized using the procedure applied for Cpd. No. 17 from Cpd. No. 105 to yield 3.0 mg, white powder, 53% yield. >98% purity. MS (ESI): m/z calculated for C₂₉H₅₀N₉O₅ [M+H]⁺ 604.39, found 604.52.

N-((3R,6S,9S,12R)-3-(cyclohexylmethyl)-6-ethyl-9-(3-(hydrazinecarboximidamido)propyl)-12-methyl-2,5,8,11-tetraoxo-1,4,7,10-tetraazacyclotetradecan-12-yl)isobutyramide (Cpd. No. 34) CF₃COOH salt. Cpd. No. 34 was synthesized using the procedure applied for Cpd. No. 17 from Cpd. No. 105 to yield 5.9 mg, white powder, 89% yield. >98% purity. MS (ESI): m/z calculated for C₂₈H₅₂N₉O₅ [M+H]⁺ 594.41, found 594.49.

N-((3R,6S,9S,12R)-9-(3-(3-carbamimidoylguanidino)propyl)-3-(cyclohexylmethyl)-6-ethyl-12-methyl-2,5,8,11-tetraoxo-1,4,7,10-tetraazacyclotetradecan-12-yl)isobutyramide (Cpd. No. 35) CF₃COOH salt. Cpd. No. 35 was synthesized using the procedure applied for Cpd. No. 17 from Cpd. No. 105 to yield 2.2 mg, white powder, 38% yield. >98% purity. MS (ESI): m/z calculated for C₂₉H₅₃N₁₀O₅ [M+H]⁺ 621.42, found 621.51.

N-((3R,6S,9S,12R)-3-(cyclohexylmethyl)-9-(3-(3,3-dimethylguanidino)propyl)-6-ethyl-12-methyl-2,5,8,11-tetraoxo-1,4,7,10-tetraazacyclotetradecan-12-yl)isobutyramide (Cpd. No. 36) CF₃COOH salt. White solid. >98% purity. MS (ESI): m/z calculated for C₃₀H₅₅N₈O₅ [M+H]⁺ 607.43, found 607.51.

N-((3R,6S,9S,12R)-3-(cyclohexylmethyl)-6-ethyl-12-methyl-2,5,8,11-tetraoxo-9-(3-((1,4,5,6-tetrahydropyrimidin-2-yl)amino)propyl)-1,4,7,10-tetraazacyclotetradecan-12-yl)isobutyramide (Cpd. No. 40) CF₃COOH salt. Cpd. No. 40 was synthesized using the procedure applied for Cpd. No. 17 from Cpd. No. 105 to yield 2.9 mg, white powder, 50% yield. >98% purity. MS (ESI): m/z calculated for C₃₁H₅₅N₈O₅ [M+H]⁺ 619.43, found 619.50.

N-((3R,6S,9S,12R)-3-(cyclohexylmethyl)-6-ethyl-9-(3-(3-ethylguanidino)propyl)-12-methyl-2,5,8,11-tetraoxo-1,4,7,10-tetraazacyclotetradecan-12-yl)isobutyramide (Cpd. No. 45) CF₃COOH salt. Cpd. No. 45 was synthesized using the procedure applied for Cpd. No. 17 from Cpd. No. 105 to yield 7.3 mg, white powder, 92% yield. >98% purity. MS (ESI): m/z calculated for C₃₀H₅₅N₈O₅ [M+H]⁺ 607.43, found 607.56.

N-((3R,6S,9S,12R)-3-(cyclohexylmethyl)-6-ethyl-12-methyl-2,5,8,11-tetraoxo-9-(3-(3-propylguanidino)propyl)-1,4,7,10-tetraazacyclotetradecan-12-yl)isobutyramide (Cpd. No. 46) CF₃COOH salt. Cpd. No. 46 was synthesized using the procedure applied for Cpd. No. 17 from Cpd. No. 105 to yield 4.6 mg, white powder, 80% yield. >98% purity. MS (ESI): m/z calculated for C₃₁H₅₇N₈O₅ [M+H]⁺ 621.45, found 621.56.

N-((3R,6S,9S,12R)-9-(3-(3-butylguanidino)propyl)-3-(cyclohexylmethyl)-6-ethyl-12-methyl-2,5,8,11-tetraoxo-1,4,7,10-tetraazacyclotetradecan-12-yl)isobutyramide (Cpd. No. 47) CF₃COOH salt. Cpd. No. 47 was synthesized using the procedure applied for Cpd. No. 17 from Cpd. No. 105 to yield 3.9 mg, white powder, 66% yield. >98% purity. MS (ESI): m/z calculated for C₃₂H₅₉N₈O₅ [M+H]⁺ 635.46, found 635.59.

N-((3R,6S,9S,12R)-3-(cyclohexylmethyl)-6-ethyl-12-methyl-9-(3-(4-methylpiperazin-1-yl)propyl)-2,5,8,11-tetraoxo-1,4,7,10-tetraazacyclotetradecan-12-yl)isobutyramide (Cpd. No. 43). Cpd. No. 43 was synthesized using the procedure applied for Cpd. No. 17 from Cpd. No. 105 to yield 4.2 mg, white powder, 73% yield LC-MS(ESI) m/z (M+H)⁺: 620.55; calcd for C₃₂H₅₈N₇O₅ (M+H)⁺: 620.45; >98% purity.

N-((3R,6S,9S,12R)-9-(3-((amino(methylamino)methyl)amino)propyl)-3-(cyclohexylmethyl)-6-ethyl-12-methyl-2,5,8,11-tetraoxo-1,4,7,10-tetraazacyclotetradecan-12-yl)isobutyramide (Cpd. No. 87). Cpd. No. 87 was synthesized using the procedure applied for Cpd. No. 17 from Cpd. No. 105 to yield 3.4 mg, white powder, 62% yield. LC-MS(ESI) m/z (M+H)⁺: 593.50; calcd for C₂₉H₅₅N₈O₅ (M+H)⁺: 595.43; >98% purity.

N-((3R,6S,9S,12R)-6-ethyl-9-(3-guanidinopropyl)-3,12-dimethyl-2,5,8,11-tetraoxo-3-phenyl-1,4,7,10-tetraazacyclotetradecan-12-yl)isobutyramide (Cpd. No. 78). Cpd. No. 78 was synthesized using the procedure applied for Cpd. No. 4 to yield 2.5 mg, white powder, 5% yield over 4 steps. LC-MS(ESI) m/z (M+H)⁺: 573.32; calcd for C₂₈H₄₅N₈O₅ (M+H)⁺: 573.35; >98% purity.

N-((3R,6S,9S,12R)-9-(3-guanidinopropyl)-12-methyl-2,5,8,11-tetraoxo-3-phenyl-6-propyl-1,4,7,10-tetraazacyclotetradecan-12-yl)isobutyramide (Cpd. No. 88). Cpd. No. 88 was synthesized using the procedure applied for Cpd. No. 4 to yield 19.6 mg, white powder, 34% yield over 4 steps. LC-MS(ESI) m/z (M+H)⁺: 573.33; calcd for C₂₈H₄₅N₈O₅ (M+H)⁺: 573.35; >98% purity. ¹H NMR (400 MHz, Methanol-d₄) δ 8.64 (s, 1H), 8.38 (d, J=4.0 Hz, 1H), 8.19 (d, J=9.5 Hz, 1H), 8.03 (d, J=9.2 Hz, 1H), 7.93-7.86 (m, 1H), 7.45-7.39 (m, 2H), 7.36-7.25 (m, 3H), 5.46 (d, J=9.2 Hz, 1H), 4.47 (ddd, J=11.0, 9.5, 3.6 Hz, 1H), 4.15 (ddd, J=7.5, 6.3, 4.0 Hz, 1H), 3.83-3.68 (m, 1H), 3.30-3.15 (m, 2H), 2.75-2.57 (m, 3H), 2.18-2.05 (m, 1H), 1.91-1.65 (m, 5H), 1.53 (s, 3H), 1.47-1.26 (m, 4H), 1.23 (d, J=6.9 Hz, 3H), 1.16 (d, J=6.6 Hz, 3H), 0.93 (t, J=7.3 Hz, 3H).

N-((3R,6S,9S,12R)-9-(3-guanidinopropyl)-6-isopropyl-12-methyl-2,5,8,11-tetraoxo-3-phenyl-1,4,7,10-tetraazacyclotetradecan-12-yl)isobutyramide (Cpd. No. 89). Cpd. No. 89 was synthesized using the procedure applied for Cpd. No. 4 to yield 11.7 mg, white powder, 20% yield over 4 steps. LC-MS(ESI) m/z (M+H)⁺: 573.43; calcd for C₂₈H₄₅N₈O₅ (M+H)⁺: 573.35; >98% purity. ¹H NMR (400 MHz, Methanol-d₄) δ 8.53 (s, 1H), 8.23 (d, J=3.6 Hz, 1H), 7.98 (t, J=9.7 Hz, 2H), 7.93-7.85 (m, 1H), 7.45-7.36 (m, 2H), 7.36-7.24 (m, 3H), 5.47 (d, J=9.0 Hz, 1H), 4.59 (dd, J=10.2, 3.9 Hz, 1H), 4.15 (ddd, J=7.7, 6.3, 3.5 Hz, 1H), 3.75 (td, J=13.1, 10.9, 3.2 Hz, 1H), 3.28-3.14 (m, 2H), 2.76-2.61 (m, 3H), 2.61-2.50 (m, 1H), 1.98-1.66 (m, 4H), 1.52 (s, 3H), 1.38-1.29 (m, 1H), 1.21 (d, J=6.9 Hz, 3H), 1.18 (d, J=6.7 Hz, 3H), 1.03 (d, J=6.9 Hz, 3H), 0.92 (d, J=6.9 Hz, 3H).

N-((3R,6S,9S,12R)-6-((R)-sec-butyl)-9-(3-guanidinopropyl)-12-methyl-2,5,8,11-tetraoxo-3-phenyl-1,4,7,10-tetraazacyclotetradecan-12-yl)isobutyramide (Cpd. No. 90). Cpd. No. 90 was synthesized using the procedure applied for Cpd. No. 4 to yield 7.8 mg, white powder, 14% yield over 4 steps. LC-MS(ESI) m/z (M+H)⁺: 587.44; calcd for C₂₉H₄₇N₈O₅ (M+H)⁺: 587.37; >98% purity. ¹H NMR (400 MHz, Methanol-d₄) δ 8.53 (s, 1H), 8.22 (d, J=3.6 Hz, 1H), 8.01 (d, J=9.0 Hz, 1H), 7.92 (t, J=9.7 Hz, 2H), 7.42-7.35 (m, 2H), 7.35-7.23 (m, 3H), 5.46 (d, J=9.0 Hz, 1H), 4.61 (dd, J=10.2, 3.7 Hz, 1H), 4.14 (ddd, J=7.7, 6.3, 3.5 Hz, 1H), 3.75 (td, J=9.6, 5.3 Hz, 1H), 3.28-3.13 (m, 2H), 2.77-2.57 (m, 3H), 2.36-2.23 (m, 1H), 1.94-1.58 (m, 5H), 1.52 (s, 3H), 1.36-1.25 (m, 2H), 1.22 (d, J=6.9 Hz, 3H), 1.18 (d, J=6.7 Hz, 3H), 0.97-0.89 (m, 6H).

N-((3R,6S,9S,12R)-9-(3-(3-acetylguanidino)propyl)-6-ethyl-12-methyl-2,5,8,11-tetraoxo-3-phenyl-1,4,7,10-tetraazacyclotetradecan-12-yl)isobutyramide (Cpd. No. 91). Cpd. No. 91 was synthesized using the procedure applied for Cpd. No. 17 from Cpd. No. 103 to yield 3.4 mg, white powder, 61% yield. LC-MS(ESI) m/z (M+H)⁺: 601.44; calcd for C₂₉H₄₄N₈O₆ (M+H)⁺: 600.34; >98% purity. ¹H NMR (400 MHz, Methanol-d₄) δ 8.60 (s, 1H), 8.33 (d, J=4.0 Hz, 1H), 8.21 (d, J=9.5 Hz, 1H), 7.99 (d, J=9.2 Hz, 1H), 7.87 (d, J=9.2 Hz, 1H), 7.49-7.37 (m, 2H), 7.37-7.20 (m, 3H), 5.46 (d, J=9.2 Hz, 1H), 4.39 (td, J=10.1, 3.8 Hz, 1H), 4.22-4.14 (m, 1H), 3.82-3.71 (m, 1H), 3.46-3.34 (m, 2H), 2.75-2.58 (m, 3H), 2.25-2.08 (m, 4H), 1.97-1.73 (m, 5H), 1.53 (s, 3H), 1.40-1.33 (m, 1H), 1.22 (d, J=6.9 Hz, 3H), 1.16 (d, J=6.7 Hz, 3H), 0.95 (t, J=7.4 Hz, 3H).

N-((3R,6S,9S,12R)-6-ethyl-12-methyl-2,5,8,11-tetraoxo-3-phenyl-9-(3-((1,4,5,6-tetrahydropyrimidin-2-yl)amino)propyl)-1,4,7,10-tetraazacyclotetradecan-12-yl)isobutyramide (Cpd. No. 92). Cpd. No. 92 was synthesized using the procedure applied for Cpd. No. 17 from Cpd. No. 103 to yield 7.2 mg, white powder, 65% yield. LC-MS(ESI) m/z (M+H)⁺: 599.46; calcd for C₃₀H₄₇N₈O₅ (M+H)⁺: 599.37; >98% purity. ¹H NMR (400 MHz, Methanol-d₄) δ 8.60 (s, 1H), 8.33 (d, J=4.1 Hz, 1H), 8.20 (d, J=9.5 Hz, 1H), 7.99 (d, J=9.2 Hz, 1H), 7.93-7.82 (m, 1H), 7.48-7.37 (m, 2H), 7.37-7.23 (m, 3H), 5.46 (d, J=9.1 Hz, 1H), 4.38 (td, J=10.1, 3.8 Hz, 1H), 4.15 (td, J=7.1, 4.0 Hz, 1H), 3.81-3.71 (m, 1H), 3.38-3.32 (m, 4H), 3.26-3.08 (m, 2H), 2.76-2.57 (m, 3H), 2.26-2.10 (m, 1H), 1.98-1.90 (m, 2H), 1.90-1.65 (m, 5H), 1.53 (s, 3H), 1.42-1.32 (m, 1H), 1.22 (d, J=6.9 Hz, 3H), 1.16 (d, J=6.6 Hz, 3H), 0.95 (t, J=7.4 Hz, 3H).

N-((3R,6S,9S,12R)-6-ethyl-3-(3-fluorophenyl)-9-(3-guanidinopropyl)-12-methyl-2,5,8,11-tetraoxo-1,4,7,10-tetraazacyclotetradecan-12-yl)isobutyramide (Cpd. No. 80). Cpd. No. 80 was synthesized using the procedure applied for Cpd. No. 4 to yield 8.0 mg, white powder, 14% yield over 4 steps. LC-MS(ESI) m/z (M+H)⁺: 577.34; calcd for C₂₇H₄₂FN₈O₅ (M+H)⁺: 577.33; >98% purity. ¹H NMR (400 MHz, Methanol-d₄) δ 8.64 (s, 1H), 8.38 (d, J=3.9 Hz, 1H), 8.16 (d, J=9.5 Hz, 1H), 8.06 (d, J=9.2 Hz, 1H), 7.86 (dd, J=9.9, 2.9 Hz, 1H), 7.34 (td, J=8.0, 5.9 Hz, 1H), 7.21 (dt, J=7.7, 1.2 Hz, 1H), 7.17 (dt, J=9.8, 2.1 Hz, 1H), 7.11-6.91 (m, 1H), 5.49 (d, J=9.2 Hz, 1H), 4.39 (td, J=10.1, 3.6 Hz, 1H), 4.17 (td, J=7.5, 7.0, 3.9 Hz, 1H), 3.85-3.64 (m, 1H), 3.28-3.15 (m, 2H), 2.76-2.55 (m, 3H), 2.19 (ddd, J=14.2, 7.4, 3.7 Hz, 1H), 1.87-1.66 (m, 5H), 1.53 (s, 3H), 1.37 (dd, J=6.7, 3.7 Hz, 1H), 1.22 (d, J=6.9 Hz, 3H), 1.16 (d, J=6.6 Hz, 3H), 0.96 (t, J=7.4 Hz, 3H).

N-((8S,11R,16R)-8-(3-guanidinopropyl)-11-methyl-7,10,15,18-tetraoxo-16-phenyl-6,9,14,17-tetraazaspiro[4.13]octadecan-11-yl)isobutyramide (Cpd. No. 86). Cpd. No. 86 was synthesized using the procedure applied for Cpd. No. 4 to yield 4.2 mg, white powder, 7% yield over 4 steps. LC-MS(ESI) m/z (M+H)⁺: 585.43; calcd for C₂₉H₄₅N₈O₅ (M+H): 585.35; >98% purity.

N-((3R,6S,9S,12R)-6-ethyl-9-(3-guanidinopropyl)-12-methyl-3-(naphthalen-1-yl)-2,5,8,11-tetraoxo-1,4,7,10-tetraazacyclotetradecan-12-yl)isobutyramide (Cpd. No. 81). Cpd. No. 81 was synthesized using the procedure applied for Cpd. No. 4 to yield 3.3 mg, white powder, 6% yield over 4 steps. LC-MS(ESI) m/z (M+H)⁺: 609.35; calcd for C₃₁H₄₅N₈O₅ (M+H)⁺: 609.35; >98% purity.

N-((3R,6S,9S,12R)-6-ethyl-9-(3-guanidinopropyl)-12-methyl-3-(naphthalen-2-yl)-2,5,8,11-tetraoxo-1,4,7,10-tetraazacyclotetradecan-12-yl)isobutyramide (Cpd. No. 82). Cpd. No. 82 was synthesized using the procedure applied for Cpd. No. 4 to yield 9 mg, white powder, 15% yield over 4 steps. LC-MS(ESI) m/z (M+H)⁺: 609.42; calcd for C₃₁H₄₅N₈O₅ (M+H)⁺: 609.35; >98% purity. ¹H NMR (400 MHz, Methanol-d₄) δ 8.63 (s, 1H), 8.37 (d, J=4.0 Hz, 1H), 8.22 (d, J=9.5 Hz, 1H), 8.13 (d, J=9.2 Hz, 1H), 7.94 (d, J=8.5 Hz, 1H), 7.89-7.78 (m, 4H), 7.58 (dd, J=8.5, 1.8 Hz, 1H), 7.52-7.38 (m, 2H), 5.66 (d, J=9.1 Hz, 1H), 4.40 (td, J=10.1, 3.7 Hz, 1H), 4.19 (td, J=7.1, 4.0 Hz, 1H), 3.88-3.73 (m, 1H), 3.28-3.11 (m, 2H), 2.79-2.55 (m, 3H), 2.28-2.10 (m, 1H), 1.92-1.68 (m, 5H), 1.55 (s, 3H), 1.43-1.34 (m, 1H), 1.24 (d, J=6.9 Hz, 3H), 1.18 (d, J=6.7 Hz, 3H), 0.97 (t, J=7.4 Hz, 3H).

N-((3R,6S,9S,12R)-6-ethyl-3-(2-fluorophenyl)-9-(3-guanidinopropyl)-12-methyl-2,5,8,11-tetraoxo-1,4,7,10-tetraazacyclotetradecan-12-yl)isobutyramide (Cpd. No. 79). Cpd. No. 79 was synthesized using the procedure applied for Cpd. No. 4 to yield 10.5 mg, white powder, 18% yield over 4 steps. LC-MS(ESI) m/z (M+H)⁺: 577.39; calcd for C₂₇H₄₂FN₈O₅ (M+H)⁺: 577.33; >98% purity. ¹H NMR (400 MHz, Methanol-d₄) δ 8.62 (s, 1H), 8.35 (d, J=3.9 Hz, 1H), 8.18 (d, J=9.5 Hz, 1H), 8.07 (d, J=9.2 Hz, 1H), 7.89-7.73 (m, 1H), 7.43 (td, J=7.5, 1.7 Hz, 1H), 7.36-7.26 (m, 1H), 7.14 (td, J=7.6, 1.2 Hz, 1H), 7.08 (ddd, J=9.6, 8.3, 1.2 Hz, 1H), 5.84 (d, J=9.2 Hz, 1H), 4.41 (td, J=10.1, 3.7 Hz, 1H), 4.15 (td, J=7.5, 7.0, 3.9 Hz, 1H), 3.87-3.65 (m, 1H), 3.22 (ddt, J=13.6, 11.4, 7.1 Hz, 2H), 2.79-2.50 (m, 3H), 2.19 (m, J=14.8, 7.5, 3.8 Hz, 1H), 1.93-1.67 (m, 5H), 1.52 (s, 3H), 1.36-1.28 (m, 1H), 1.22 (d, J=6.9 Hz, 3H), 1.16 (d, J=6.6 Hz, 3H), 0.96 (t, J=7.4 Hz, 3H).

N-((3R,6S,9S,12R)-6-ethyl-9-(3-guanidinopropyl)-3-(4-methoxyphenyl)-12-methyl-2,5,8,11-tetraoxo-1,4,7,10-tetraazacyclotetradecan-12-yl)isobutyramide (Cpd. No. 93). Cpd. No. 93 was synthesized using the procedure applied for Cpd. No. 4 to yield 6.0 mg, white powder, 10% yield over 4 steps. LC-MS(ESI) m/z (M+H)⁺: 589.38; calcd for C₂₈H₄₅N₈O₆ (M+H)⁺: 589.35; >98% purity. ¹H NMR (400 MHz, Methanol-d₄) δ 8.59 (s, 1H), 8.32 (d, J=4.1 Hz, 1H), 8.20 (d, J=9.5 Hz, 1H), 7.92 (d, J=9.2 Hz, 1H), 7.85 (d, J=9.2 Hz, 1H), 7.39-7.25 (m, 2H), 6.94-6.83 (m, 2H), 5.41 (d, J=9.2 Hz, 1H), 4.37 (td, J=10.1, 3.8 Hz, 1H), 4.15 (td, J=7.5, 7.0, 4.0 Hz, 1H), 3.91-3.61 (m, 4H), 3.28-3.12 (m, 2H), 2.74-2.52 (m, 3H), 2.26-2.11 (m, 1H), 1.96-1.69 (m, 5H), 1.52 (s, 3H), 1.40-1.31 (m, 1H), 1.22 (d, J=6.9 Hz, 3H), 1.16 (d, J=6.6 Hz, 3H), 0.95 (t, J=7.4 Hz, 3H).

N-((3R,6S,9S,12R)-6-ethyl-9-(3-guanidinopropyl)-3-(3-methoxyphenyl)-12-methyl-2,5,8,11-tetraoxo-1,4,7,10-tetraazacyclotetradecan-12-yl)isobutyramide (Cpd. No. 94). Cpd. No. 94 was synthesized using the procedure applied for Cpd. No. 17 to yield 2.3 mg, white powder, 42% yield. LC-MS(ESI) m/z (M+H)⁺: 589.36; calcd for C₂₈H₄₅N₈O₆ (M+H)⁺: 589.35; >98% purity. ¹H NMR (400 MHz, Methanol-d₄) δ 8.59 (s, 1H), 8.32 (d, J=4.1 Hz, 1H), 8.19 (d, J=9.5 Hz, 1H), 7.96 (d, J=9.2 Hz, 1H), 7.87 (d, J=9.2 Hz, 1H), 7.22 (t, J=7.9 Hz, 1H), 7.08-7.01 (m, 1H), 6.97 (dt, J=7.6, 1.2 Hz, 1H), 6.89-6.77 (m, 1H), 5.42 (d, J=9.1 Hz, 1H), 4.38 (td, J=10.1, 3.8 Hz, 1H), 4.16 (td, J=7.5, 7.1, 4.1 Hz, 1H), 3.91-3.65 (m, 4H), 3.29-3.15 (m, 2H), 2.78-2.54 (m, 3H), 2.18 (ddd, J=14.1, 7.4, 3.9 Hz, 1H), 1.94-1.62 (m, 5H), 1.53 (s, 3H), 1.41-1.34 (m, 1H), 1.28-1.20 (m, 3H), 1.16 (d, J=6.7 Hz, 3H), 0.95 (t, J=7.4 Hz, 3H).

N-((3R,6S,9S,12R)-3-(cyclohexylmethyl)-9-(3-((5,5-difluoro-1,4,5,6-tetrahydropyrimidin-2-yl)amino)propyl)-6-ethyl-12-methyl-2,5,8,11-tetraoxo-1,4,7,10-tetraazacyclotetradecan-12-yl)isobutyramide (Cpd. No. 95). Cpd. No. 95 was synthesized using the procedure applied for Cpd. No. 17 from Cpd. No. 105 to yield 1.6 mg, white powder, 44% yield. LC-MS(ESI) m/z (M+H)⁺: 655.51; calcd for C₃₁H₅₃F₂N₈O₅ (M+H)⁺: 655.41; >98% purity.

N-((3R,6S,9S,12R)-3-(cyclohexylmethyl)-9-(3-((5,5-dimethyl-1,4,5,6-tetrahydropyrimidin-2-yl)amino)propyl)-6-ethyl-12-methyl-2,5,8,11-tetraoxo-1,4,7,10-tetraazacyclotetradecan-12-yl)isobutyramide (Cpd. No. 96). Cpd. No. 96 was synthesized using the procedure applied for Cpd. No. 17 from Cpd. No. 105 to yield 9.0 mg, white powder, 93% yield. LC-MS(ESI) m/z (M+H)⁺: 647.64; calcd for C₃₃H₅₉N₈O₅ (M+H)⁺: 647.46; >98% purity.

N-((3R,6S,9S,12R)-3-(cyclohexylmethyl)-6-ethyl-12-methyl-9-(3-((5-methyl-1,4,5,6-tetrahydropyrimidin-2-yl)amino)propyl)-2,5,8,11-tetraoxo-1,4,7,10-tetraazacyclotetradecan-12-yl)isobutyramide (Cpd. No. 97). Cpd. No. 97 was synthesized using the procedure applied for Cpd. No. 17 from Cpd. No. 105 to yield 5.1 mg, white powder, 87% yield. LC-MS(ESI) m/z (M+H)⁺: 633.63; calcd for C₃₂H₅₇N₈O₅ (M+H)⁺: 633.45; >98% purity.

N-((3R,6S,9S,12R)-6-ethyl-12-methyl-3-(naphthalen-2-yl)-2,5,8,11-tetraoxo-9-(3-((1,4,5,6-tetrahydropyrimidin-2-yl)amino)propyl)-1,4,7,10-tetraazacyclotetradecan-12-yl)isobutyramide (Cpd. No. 98). Cpd. No. 98 was synthesized using the procedure applied for Cpd. No. 17 from Cpd. No. 103 to yield 3.9 mg, white powder, 68% yield. LC-MS(ESI) m/z (M+H)⁺: 649.45; calcd for C₃₄H₄₉N₈O₅ (M+H)⁺: 649.38; >98% purity. ¹H NMR (400 MHz, Methanol-d₄) δ 8.64 (s, 1H), 8.38 (d, J=4.1 Hz, 1H), 8.22 (d, J=9.5 Hz, 1H), 8.12 (d, J=9.1 Hz, 1H), 7.93 (d, J=8.8 Hz, 1H), 7.89-7.77 (m, 4H), 7.58 (dd, J=8.5, 1.7 Hz, 1H), 7.51-7.44 (m, 2H), 5.65 (d, J=9.1 Hz, 1H), 4.40 (td, J=10.1, 3.7 Hz, 1H), 4.19 (td, J=7.0, 3.9 Hz, 1H), 3.80 (t, J=11.2 Hz, 1H), 3.37-3.32 (m, 4H), 3.26-3.10 (m, 2H), 2.78-2.59 (m, 3H), 2.27-2.17 (m, 1H), 1.97-1.65 (m, 7H), 1.55 (s, 3H), 1.40-1.34 (m, 1H), 1.24 (d, J=6.9 Hz, 3H), 1.17 (d, J=6.6 Hz, 3H), 0.96 (t, J=7.5 Hz, 3H).

N-((3R,6S,9S,12R)-3-benzyl-6-ethyl-9-(3-guanidinopropyl)-12-methyl-2,5,8,11-tetraoxo-1,4,7,10-tetraazacyclotetradecan-12-yl)isobutyramide (Cpd. No. 54) CF₃COOH salt. Solid (56% yield over 4 steps). MS (ESI): m/z calculated for C₂₈H₄₄N₈O₅ [M+H]⁺ 572.34, found 573.35. ¹H NMR (400 MHz, Methanol-d₄) δ 8.52 (s, 1H), 8.22 (d, J=4.4 Hz, 1H), 8.15 (d, J=9.6 Hz, 1H), 7.33 (d, J=9.6 Hz, 1H), 7.25-7.14 (m, 5H), 4.66-4.59 (m, 1H), 4.24-4.14 (m, 1H), 4.13-4.09 (m, 1H), 3.82-3.72 (m, 1H), 3.48-3.42 (m, 1H), 3.30-3.15 (m, 1H), 2.75-2.55 (m, 4H), 2.07-1.97 (m, 1H), 1.90-1.82 (m, 1H), 1.80-1.60 (m, 4H), 1.50 (s, 3H), 1.38-1.28 (m, 1H), 1.18 (d, J=6.8 Hz, 3H), 1.11 (d, J=6.8 Hz, 3H), 0.86 (t, J=7.6 Hz, 3H).

N-((3R,6S,9S,12R)-6-ethyl-9-(3-guanidinopropyl)-3-isobutyl-12-methyl-2,5,8,11-tetraoxo-1,4,7,10-tetraazacyclotetradecan-12-yl)isobutyramide (Cpd. No. 49) CF₃COOH salt. Solid (35% yield over 4 steps). MS (ESI): m/z calculated for C₂₅H₄₆N₈O₅ [M+H]⁺ 538.36, found 539.28. ¹H NMR (400 MHz, Methanol-d₄) δ 8.52 (s, 1H), 8.27-8.21 (m, 2H), 7.61-7.57 (m, 1H), 7.27 (d, J=9.2 Hz, 1H), 4.53-4.38 (m, 2H), 4.16-4.08 (m, 1H), 3.68-3.78 (m, 1H), 3.28-3.18 (m, 3H), 2.72-2.64 (m, 1H), 2.60-2.51 (m, 2H), 2.22-2.12 (m, 1H), 1.92-1.82 (m, 3H), 1.78-1.68 (m, 3H), 1.65-1.55 (m, 1H), 1.54-1.42 (m, 5H), 1.38-1.28 (m, 1H), 1.18 (d, J=7.2 Hz, 3H), 1.16-1.08 (m, 5H), 0.97-0.90 (m, 9H).

N-((3R,6S,9S,12R)-3-cyclohexyl-6-ethyl-9-(3-guanidinopropyl)-12-methyl-2,5,8,11-tetraoxo-1,4,7,10-tetraazacyclotetradecan-12-yl)isobutyramide (Cpd. No. 51) CF₃COOH salt. Solid (51% yield over 4 steps). MS (ESI): m/z calculated for C₂₇H₄₈N₈O₅ [M+H]⁺ 564.37, found 565.39. ¹H NMR (400 MHz, Methanol-d₄) δ 8.54 (s, 1H), 8.25-8.17 (m, 2H), 7.64-7.58 (m, 1H), 7.28 (d, J=9.6 Hz, 1H), 4.53-4.44 (m, 1H), 4.39-4.33 (m, 1H), 4.15-4.08 (m, 1H), 3.82-3.72 (m, 1H), 3.28-3.15 (m, 3H), 2.70-2.62 (m, 1H), 2.60-2.50 (m, 2H), 2.25-2.15 (m, 2H), 1.90-1.82 (m, 2H), 1.80-1.60 (m, 9H), 1.48 (s, 3H), 1.35-1.25 (m, 4H), 1.20-1.00 (m, 10H), 0.94 (t, J=7.2 Hz, 3H).

N-((3R,6S,9S,12R)-6-ethyl-9-(3-guanidinopropyl)-3,12-dimethyl-2,5,8,11-tetraoxo-1,4,7,10-tetraazacyclotetradecan-12-yl)isobutyramide (Cpd. No. 48) CF₃COOH salt. Solid (38% yield over 4 steps). MS (ESI): m/z calculated for C₂₂H₄₀N₈O₅ [M+H]⁺ 496.31, found 497.20. ¹H NMR (400 MHz, Methanol-d₄) δ 8.50 (s, 1H), 8.24 (d, J=4 Hz, 1H), 8.15 (d, J=9.6 Hz, 1H), 7.62-7.58 (m, 1H), 7.52-7.45 (m, 1H), 4.51-4.38 (m, 2H), 4.12-4.05 (m, 1H), 3.78-3.68 (m, 1H), 3.28-3.18 (m, 3H), 2.76-2.68 (m, 1H), 2.65-2.50 (m, 2H), 2.22-2.12 (m, 1H), 1.92-1.83 (m, 2H), 1.80-1.65 (m, 4H), 1.50 (s, 3H), 1.36-1.28 (m, 6H), 1.19 (d, J=6.8 Hz, 3H), 1.14 (d, J=6.8 Hz, 3H), 0.94 (t, J=7.6 Hz, 3H).

N-((3R,6S,9S,12R)-3-cyclopentyl-6-ethyl-9-(3-guanidinopropyl)-12-methyl-2,5,8,11-tetraoxo-1,4,7,10-tetraazacyclotetradecan-12-yl)isobutyramide (Cpd. No. 50) CF₃COOH salt. Solid (31% yield over 4 steps). MS (ESI): m/z calculated for C₂₆H₄₆N₈O₅ [M+H]⁺ 550.36, found 551.46. ¹H NMR (400 MHz, Methanol-d₄) δ 8.48 (s, 1H), 8.23-8.17 (m, 2H), 7.62-7.59 (m, 1H), 7.35 (d, J=10.0 Hz, 1H), 4.55-4.44 (m, 2H), 4.11-4.07 (m, 1H), 3.75-3.71 (m, 1H), 3.28-3.15 (m, 3H), 2.78-2.48 (m, 4H), 2.28-2.15 (m, 1H), 1.92-1.83 (m, 2H), 1.82-1.62 (m, 6H), 1.58-1.45 (m, 8H), 1.38-1.35 (m, 2H), 1.34-1.23 (m, 2H), 1.18 (d, J=6.8 Hz, 3H), 1.13 (d, J=6.8 Hz, 3H), 0.94 (t, J=7.6 Hz, 3H).

N-((3R,6S,9S,12R)-6-ethyl-3-(2-fluorobenzyl)-9-(3-guanidinopropyl)-12-methyl-2,5,8,11-tetraoxo-1,4,7,10-tetraazacyclotetradecan-12-yl)isobutyramide (Cpd. No. 55) CF₃COOH salt. Solid (30% yield over 4 steps). MS (ESI): m/z calculated for C₂₈H₄₃FN₈O₅ [M+H]⁺ 590.33, found 591.47. ¹H NMR (400 MHz, Methanol-d₄) δ 8.54 (s, 1H), 8.20 (d, J=4.4 Hz, 1H), 8.13 (d, J=9.6 Hz, 1H), 7.68-7.60 (m, 1H), 7.45-7.40 (m, 1H), 7.27-7.18 (m, 2H), 7.08-6.98 (m, 2H), 4.74-4.67 (m, 1H), 4.20-4.10 (m, 1H), 3.85-3.75 (m, 1H), 3.65-3.55 (m, 1H), 3.25-3.15 (m, 3H), 2.73-2.58 (m, 4H), 2.05-1.95 (m, 1H), 1.88-1.80 (m, 2H), 1.78-1.68 (m, 2H), 1.65-1.55 (m, 2H), 1.49 (s, 3H), 1.40-1.28 (m, 4H), 1.18 (d, J=6.8 Hz, 3H), 1.11 (d, J=6.8 Hz, 3H), 0.86 (t, J=7.6 Hz, 3H).

N-((3R,6S,9S,12R)-6-ethyl-3-(3-fluorobenzyl)-9-(3-guanidinopropyl)-12-methyl-2,5,8,11-tetraoxo-1,4,7,10-tetraazacyclotetradecan-12-yl)isobutyramide (Cpd. No. 56) CF₃COOH salt. Solid (31% yield over 4 steps). MS (ESI): m/z calculated for C₂₈H₄₃FN₈O₅ [M+H]⁺ 590.33, found 591.45. ¹H NMR (400 MHz, Methanol-d₄) δ 8.54 (s, 1H), 8.21 (d, J=4.0 Hz, 1H), 8.11 (d, J=9.6 Hz, 1H), 7.65-7.58 (m, 1H), 7.35 (d, J=9.6 Hz, 1H), 7.30-7.20 (m, 1H), 7.03-6.95 (m, 2H), 6.94-6.86 (m, 1H), 4.69-4.62 (m, 1H), 4.28-4.20 (m, 1H), 4.13-4.07 (m, 1H), 3.82-3.72 (m, 1H), 3.47-3.40 (m, 1H), 3.30-3.20 (m, 2H), 2.80-2.72 (m, 1H), 2.70-2.50 (m, 3H), 2.10-2.00 (m, 1H), 1.95-1.80 (m, 3H), 1.79-1.60 (m, 4H), 1.48 (s, 3H), 1.40-1.28 (m, 3H), 1.18 (d, J=6.8 Hz, 3H), 1.11 (d, J=6.8 Hz, 3H), 0.88 (t, J=7.6 Hz, 3H).

N-((3R,6S,9S,12R)-6-ethyl-9-(3-guanidinopropyl)-12-methyl-3-(naphthalen-1-ylmethyl)-2,5,8,11-tetraoxo-1,4,7,10-tetraazacyclotetradecan-12-yl)isobutyramide (Cpd. No. 58) CF₃COOH salt. Solid (15% yield over 4 steps). MS (ESI): m/z calculated for C₃₂H₄₆N₈O₅ [M+H]⁺ 622.36, found 623.48. ¹H NMR (400 MHz, Methanol-d₄) δ 8.54 (s, 1H), 8.23-8.18 (m, 1H), 8.12 (d, J=10.0 Hz, 1H), 7.85-7.73 (m, 3H), 7.70-7.63 (m, 2H), 7.47-7.35 (m, 4H), 4.78-4.70 (m, 1H), 4.23-4.16 (m, 1H), 4.15-4.07 (m, 1H), 3.85-3.75 (m, 1H), 3.65-3.58 (m, 1H), 3.30-3.20 (m, 2H), 2.95-2.85 (m, 1H), 2.70-2.52 (m, 3H), 2.00-1.93 (m, 1H), 1.88-1.81 (m, 2H), 1.78-1.65 (m, 2H), 1.64-1.55 (m, 1H), 1.48 (s, 3H), 1.40-1.30 (m, 1H), 1.18 (d, J=6.8 Hz, 3H), 1.11 (d, J=6.8 Hz, 3H), 0.83 (t, J=7.6 Hz, 3H).

N-((3R,6S,9S,12R)-3-(cyclohexylmethyl)-6-ethyl-9-(3-guanidinopropyl)-12-methyl-2,5,8,11-tetraoxo-1,4,7,10-tetraazacyclotetradecan-12-yl)isobutyramide (Cpd. No. 52) CF₃COOH salt. Solid (34% yield over 4 steps). MS (ESI): m/z calculated for C₂₈H50N₈O₅ [M+H]⁺ 578.39, found 579.47. ¹H NMR (400 MHz, Methanol-d₄) δ 8.59 (s, 1H), 8.30-8.20 (m, 2H), 7.59 (d, J=8.4 Hz, 1H), 7.28 (d, J=9.6 Hz, 1H), 4.54-4.47 (m, 1H), 4.46-4.39 (m, 1H), 4.14-4.08 (m, 1H), 3.80-3.67 (m, 1H), 3.30-3.18 (m, 3H), 2.73-2.63 (m, 1H), 2.61-2.50 (m, 2H), 2.23-2.12 (m, 1H), 1.98-1.80 (m, 4H), 1.79-1.60 (m, 8H), 1.51-1.38 (m, 4H), 1.37-1.15 (m, 9H), 1.12 (d, J=6.8 Hz, 3H), 1.15-0.85 (m, 6H).

N-((3R,6S,9S,12R)-6-ethyl-3-(4-fluorophenyl)-9-(3-guanidinopropyl)-12-methyl-2,5,8,11-tetraoxo-1,4,7,10-tetraazacyclotetradecan-12-yl)isobutyramide (Cpd. No. 53) CF₃COOH salt. Solid (10% yield over 4 steps). MS (ESI): m/z calculated for C₂₇H₄₁FN₈O₅ [M+H]⁺ 576.32, found 577.34. ¹H NMR (400 MHz, Methanol-d₄) δ 8.61 (s, 1H), 8.35-8.30 (m, 1H), 8.16 (d, J=9.6 Hz, 1H), 8.02 (d, J=9.2 Hz, 1H), 7.90-7.82 (m, 1H), 7.44-7.39 (m, 2H), 7.07-7.02 (m, 2H), 5.47 (d, J=8.8 Hz, 1H), 4.44-4.35 (m, 1H), 4.20-4.10 (m, 1H), 3.80-3.72 (m, 1H), 3.30-3.17 (m, 3H), 2.80-2.60 (m, 3H), 2.24-2.12 (m, 1H), 1.92-1.68 (m, 6H), 1.53 (s, 3H), 1.38-1.32 (m, 1H), 1.22 (d, J=6.8 Hz, 3H), 1.20-1.10 (m, 4H), 0.96 (t, J=7.6 Hz, 3H).

N-((3R,6S,9S,12R)-6-ethyl-3-(4-fluorobenzyl)-9-(3-guanidinopropyl)-12-methyl-2,5,8,11-tetraoxo-1,4,7,10-tetraazacyclotetradecan-12-yl)isobutyramide (Cpd. No. 57) CF₃COOH salt. Solid (27% yield over 4 steps). MS (ESI): m/z calculated for C₂₈H₄₃FN₈O₅ [M+H]⁺ 590.33, found 591.43. ¹H NMR (400 MHz, Methanol-d₄) δ 8.55 (s, 1H), 8.23 (d, J=4.4 Hz, 1H), 8.10 (d, J=9.6 Hz, 1H), 7.65-7.60 (m, 1H), 7.34 (d, J=9.6 Hz, 1H), 7.21-7.16 (m, 2H), 7.00-6.90 (m, 2H), 4.65-4.57 (m, 1H), 4.30-4.20 (m, 1H), 4.12-4.06 (m, 1H), 3.83-3.73 (m, 1H), 3.43-3.36 (m, 1H), 3.30-3.15 (m, 3H), 2.80-2.72 (m, 1H), 2.71-2.50 (m, 3H), 2.10-2.00 (m, 1H), 1.90-1.80 (m, 2H), 1.79-1.60 (m, 4H), 1.48 (s, 3H), 1.35-1.27 (m, 1H), 1.17 (d, J=6.8 Hz, 3H), 1.11 (d, J=6.8 Hz, 3H), 0.88 (t, J=7.2 Hz, 3H).

N-((3R,6S,9S,12R)-6-ethyl-9-(3-guanidinopropyl)-3-(2-methoxyphenyl)-12-methyl-2,5,8,11-tetraoxo-1,4,7,10-tetraazacyclotetradecan-12-yl)isobutyramide (Cpd. No. 99) CF₃COOH salt. Solid (25% yield over 4 steps). MS (ESI): m/z calculated for C₂₈H44N₈O₆ [M+H]⁺ 588.34, found 589.25. ¹H NMR (400 MHz, Methanol-d₄) δ 8.51 (s, 1H), 8.32 (d, J=9.6 Hz, 1H), 8.19 (d, J=4 Hz, 1H), 7.89 (d, J=9.2 Hz), 7.32-7.22 (m, 2H), 7.00-6.89 (m, 2H), 5.76 (d, J=9.2 Hz, 1H), 4.45-4.35 (m, 1H), 4.25-4.15 (m, 1H), 3.85 (s, 3H), 3.82-3.72 (m, 1H), 3.40-3.15 (m, 3H), 2.75-2.60 (m, 2H), 2.20-2.15 (m, 1H), 1.80-1.70 (m, 4H), 1.79-1.60 (m, 4H), 1.53 (s, 3H), 1.38-1.32 (m, 1H), 1.22 (d, J=6.8 Hz, 3H), 1.20-1.10 (m, 4H), 0.96 (t, J=7.6 Hz, 3H).

N-((3R,6S,9S,12R)-3-(2-chlorophenyl)-6-ethyl-9-(3-guanidinopropyl)-12-methyl-2,5,8,11-tetraoxo-1,4,7,10-tetraazacyclotetradecan-12-yl)isobutyramide (Cpd. No. 83) CF₃COOH salt. Solid (26% yield over 4 steps). MS (ESI): m/z calculated for C₂₇H₄₁C₁N₈O₅ [M+H]⁺ 590.33, found 593.29. ¹H NMR (400 MHz, Methanol-d₄) δ 8.40 (s, 2H), 8.24 (d, J=5.2 Hz, 1H), 7.99 (d, J=9.2 Hz, 1H), 7.90-7.85 (m, 1H), 7.55-7.45 (m, 1H), 7.35-7.25 (m, 3H), 7.20-7.24 (m, 1H), 5.25-5.23 (m, 1H), 4.35-4.25 (m, 1H), 4.10-3.99 (m, 1H), 3.40-3.05 (m, 3H), 2.70-2.55 (m, 3H), 2.05-1.60 (m, 8H), 1.25-1.15 (m, 6H), 0.86 (t, J=7.6 Hz, 3H).

N-((6S,9S,12R)-3-(3-chlorophenyl)-6-ethyl-9-(3-guanidinopropyl)-12-methyl-2,5,8,11-tetraoxo-1,4,7,10-tetraazacyclotetradecan-12-yl)isobutyramide (Cpd. No. 84) CF₃COOH salt. Solid (15% yield over 4 steps). MS (ESI): m/z calculated for C₂₇H₄₁ClN₈O₅ [M+H]⁺ 590.33, found 593.29. ¹H NMR (400 MHz, Methanol-d₄) δ 8.60 (s, 1H), 8.31 (d, J=4.0 Hz, 1H), 8.21 (d, J=9.2 Hz, 1H), 8.10 (d, J=8.8 Hz, 1H), 7.80-7.70 (m, 1H), 7.51-7.48 (m, 1H), 7.42-7.41 (m, 1H), 7.32-7.28 (m, 2H), 6.05 (d, J=9.2 Hz, 1H), 4.43-4.39 (m, 1H), 4.19-4.18 (m, 1H), 3.85-3.70 (m, 1H), 3.33-3.25 (m, 2H), 2.78-2.62 (m, 3H), 2.28-2.15 (m, 1H), 1.90-1.70 (m, 5H), 1.54 (s, 3H), 1.40-1.32 (m, 1H), 1.25 (d, J=6.8 Hz, 3H), 1.20 (d, J=6.8 Hz, 3H), 0.97 (t, J=7.2 Hz, 3H).

N-((3R,6S,9S,12R)-3-(4-chlorophenyl)-6-ethyl-9-(3-guanidinopropyl)-12-methyl-2,5,8,11-tetraoxo-1,4,7,10-tetraazacyclotetradecan-12-yl)isobutyramide (Cpd. No. 100) CF₃COOH salt. Solid (20% yield over 4 steps). MS (ESI): m/z calculated for C₂₇H₄₁CN₈O₅ [M+H]⁺ 590.33, found 593.29. ¹H NMR (400 MHz, Methanol-d₄) δ 8.40-8.39 (m, 2H), 8.23-8.21 (d, J=6 Hz, 1H), 8.00 (d, J=9.2 Hz, 1H), 7.87-7.85 (m, 1H), 7.51-7.49 (m, 1H), 7.33 (d, J=8.4 Hz, 2H), 7.26 (d, J=8.4 Hz, 2H), 5.24 (s, 1H), 4.32-4.27 (m, 1H), 4.04-4.03 (m, 1H), 3.35-3.20 (m, 2H), 3.18-3.08 (m, 1H), 2.70-2.55 (m, 1H), 2.00-1.60 (m, 8H), 1.56 (s, 3H), 1.25-1.15 (m, 6H), 1.05 (t, J=7.2 Hz, 3H).

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

1. A compound having Formula I: 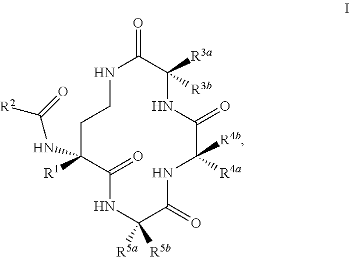
or a pharmaceutically acceptable salt or solvate thereof, wherein:
R¹ is selected from the group consisting of hydrogen, C₁₋₄ alkyl, and C₃₋₈ cycloalkyl;
R² is selected from the group consisting of C₁₋₆ alkyl, and C₃₋₈ cycloalkyl;
R^{3a} and R^{3b} are independently selected from the group consisting of hydrogen, C₁₋₆ alkyl, C₃₋₈ cycloalkyl, (cycloalkyl)alkyl, aralkyl, optionally substituted aryl, and optionally substituted heteroaryl; or
R^{3a} and R^{3b} taken together with the carbon atom to which they are attached form a C₃₋₈ cycloalkyl;
R^{4a} and R^{4b} are independently selected from the group consisting of hydrogen and C₁₋₄ alkyl; or
R^{4a} and R^{4b} taken together with the carbon atom to which they are attached form a C₃₋₈ cycloalkyl;
R^{5a} and R^{5b} are independently selected from the group consisting of hydrogen, C₁₋₄ alkyl, and —(R^{6a}R^{6b})ₘ—X—R⁹;
each R^{6a} and R^{6b} are independently selected from the group consisting of hydrogen and C₁₋₄ alkyl;
m is 1, 2, 3, 4, or 5;
X is selected from the group consisting of —N(R^{6c})—, —C(═O)NR^{6d}—, —N(R^{6e})C(═NR⁷)NR⁸—, and —N(R^{6d})C(═O)NR^{8a}—; or
X is absent;
R^{6c} is selected from the group consisting of hydrogen and C₁₋₆ alkyl;
R^{6d} is selected from the group consisting of hydrogen and C₁₋₆ alkyl;
R^{6e} is selected from the group consisting of hydrogen and C₁₋₆ alkyl;
R⁷ is selected from the group consisting of hydrogen, C₁₋₆ alkyl, and C₁₋₆ haloalkyl;
R⁸ is selected from the group consisting of hydrogen and C₁₋₆ alkyl; and
R⁹ is selected from the group consisting of hydrogen, nitro, cyano, amino, C₁₋₆ alkyl, aralkyl, (heteroaryl)alkyl, optionally substituted C₃₋₇ cycloalkyl, optionally substituted 4- to 8-membered heterocyclo, optionally substituted 5- to 10-membered heteroaryl, optionally substituted C₆₋₁₀ aryl, —C(═O)R¹⁰, —C(═NH)R¹¹, and —S(═O)₂R¹²;
R¹⁰ is selected from the group consisting of C₁₋₆ alkyl, C₁₋₆ haloalkyl, and C₁₋₆ alkenyl;
R¹¹ is amino; and
R¹² is selected from the group consisting of C₁₋₆ alkyl, C₁₋₆ haloalkyl, and C₁₋₆ alkenyl.

2. The compound of claim 1, or a pharmaceutically acceptable salt or solvate thereof, wherein R^{3b} is hydrogen.

3. The compound of claim 1, or a pharmaceutically acceptable salt or solvate thereof, wherein R^{3a} is hydrogen.

4. The compound of claim 1, or a pharmaceutically acceptable salt or solvate thereof, wherein R^{4b} is hydrogen.

5. The compound of claim 1, or a pharmaceutically acceptable salt or solvate thereof, wherein R^{4a} is hydrogen.

6. The compound of claim 1, or a pharmaceutically acceptable salt or solvate thereof, wherein Rb is hydrogen.

7. The compound of claim 1, or a pharmaceutically acceptable salt or solvate thereof, wherein R^{5a} is hydrogen.

8. The compound of claim 1, or a pharmaceutically acceptable salt or solvate thereof, having Formula II: 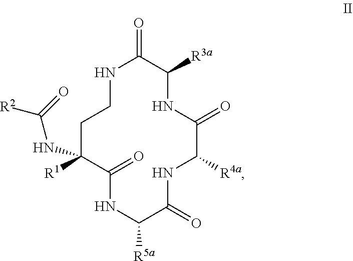
wherein R¹ is selected from the group consisting of hydrogen and methyl.

9. The compound of claim 1, or a pharmaceutically acceptable salt thereof, wherein R¹ is methyl.

10. The compound of claim 1, or a pharmaceutically acceptable salt or solvate thereof, wherein R² is C₁₋₄ alkyl.

11. The compound of claim 10, or a pharmaceutically acceptable salt or solvate thereof, wherein R² is —CH(CH₃)₂.

12. The compound of claim 1, or a pharmaceutically acceptable salt or solvate thereof, wherein R^{3a} is selected from the group consisting of:

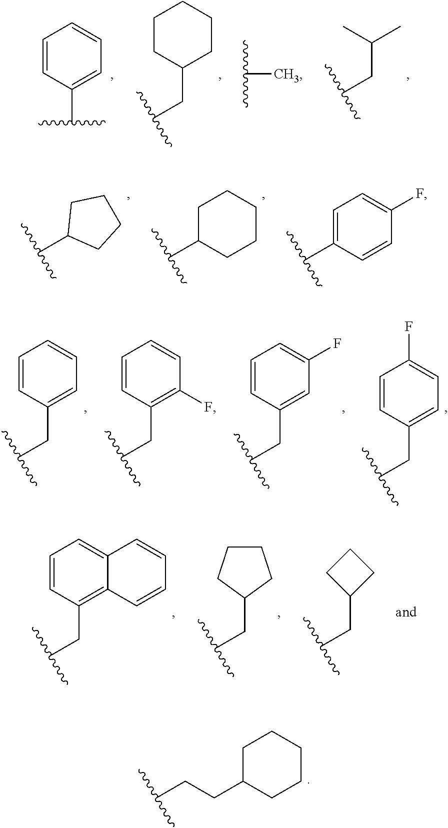

13. The compound of claim 1, or a pharmaceutically acceptable salt or solvate thereof, wherein R^{3a} is optionally substituted phenyl.

14. The compound of claim 1, or a pharmaceutically acceptable salt or solvate thereof, wherein R^{4a} is —CH₂CH₃.

15. The compound of claim 1, or a pharmaceutically acceptable salt or solvate thereof, wherein R^{5a} is —CH₂CH₂CH₂N(H)C(═NR⁷)NR⁸R⁹.

16. The compound of claim 15, or a pharmaceutically acceptable salt or solvate thereof, wherein R⁷ is hydrogen, R⁸ is hydrogen, and R⁹ is selected from the group consisting of hydrogen and C₁₋₄ alkyl.

17. The compound of claim 15, or a pharmaceutically acceptable salt or solvate thereof, wherein R^{5a} is selected from the group consisting of —CH₂CH₂CH₂N(H)C(═NH)NH₂ and —CH₂CH₂CH₂N(H)C(═NH)NHCH₃.

18. A compound, or a pharmaceutically acceptable salt or solvate thereof, selected from the group consisting of:
N-((3R,6S,9S,12R)-6-ethyl-9-(3-guanidinopropyl)-2,5,8,11-tetraoxo-3-phenyl-1,4,7,10-tetraazacyclotetradecan-12-yl)isobutyramide;
N-((3R,6S,9S,12R)-6-ethyl-9-(3-guanidinopropyl)-12-methyl-2,5,8,11-tetraoxo-3-phenyl-1,4,7,10-tetraazacyclotetradecan-12-yl)isobutyramide;
N-((3R,6S,9S,12R)-6-ethyl-12-methyl-9-(3-(3-methylguanidino)propyl)-2,5,8,11-tetraoxo-3-phenyl-1,4,7,10-tetraazacyclotetradecan-12-yl)isobutyramide;
N-((3R,6S,9S,12R)-9-(4-(dimethylamino)butyl)-6-ethyl-12-methyl-2,5,8,11-tetraoxo-3-phenyl-1,4,7,10-tetraazacyclotetradecan-12-yl)isobutyramide;
N-((3R,6S,9S,12R)-6-ethyl-12-methyl-9-(((1-methylpiperidin-4-yl)amino)methyl)-2,5,8,11-tetraoxo-3-phenyl-1,4,7,10-tetraazacyclotetradecan-12-yl)isobutyramide;
N-((3R,6S,9S,12R)-6-ethyl-12-methyl-9-(2-((1-methylpiperidin-4-yl)amino)ethyl)-2,5,8,11-tetraoxo-3-phenyl-1,4,7,10-tetraazacyclotetradecan-12-yl)isobutyramide;
N-((3R,6S,9S,12R)-6-ethyl-12-methyl-9-(3-((1-methylpiperidin-4-yl)amino)propyl)-2,5,8,11-tetraoxo-3-phenyl-1,4,7,10-tetraazacyclotetradecan-12-yl)isobutyramide;
N-((3R,6S,9S,12R)-6-ethyl-12-methyl-2,5,8,11-tetraoxo-3-phenyl-9-((pyridin-2-ylamino)methyl)-1,4,7,10-tetraazacyclotetradecan-12-yl)isobutyramide;
N-((3R,6S,12R)-6-ethyl-12-methyl-2,5,8,11-tetraoxo-3-phenyl-9-(2-(pyridin-2-ylamino)ethyl)-1,4,7,10-tetraazacyclotetradecan-12-yl)isobutyramide;
N-((3R,6S,9S,12R)-6-ethyl-12-methyl-2,5,8,11-tetraoxo-3-phenyl-9-(3-(pyridin-2-ylamino)propyl)-1,4,7,10-tetraazacyclotetradecan-12-yl)isobutyramide;
N-((3R,6S,12R)-6-ethyl-12-methyl-2,5,8,11-tetraoxo-9-phenethyl-3-phenyl-1,4,7,10-tetraazacyclotetradecan-12-yl)isobutyramide;
N-((3R,6S,12R)-6-ethyl-12-methyl-2,5,8,11-tetraoxo-3-phenyl-9-(3-phenylpropyl)-1,4,7,10-tetraazacyclotetradecan-12-yl)isobutyramide;
N-((3R,6S,12R)-9-(3-amino-3-oxopropyl)-6-ethyl-12-methyl-2,5,8,11-tetraoxo-3-phenyl-1,4,7,10-tetraazacyclotetradecan-12-yl)isobutyramide;
N-((3R,6S,9S,12R)-6-ethyl-12-methyl-2,5,8,11-tetraoxo-3-phenyl-9-(3-ureidopropyl)-1,4,7,10-tetraazacyclotetradecan-12-yl)isobutyramide;
N-((3R,6S,9S,12R)-9-(3-((4,5-dihydro-1H-imidazol-2-yl)amino)propyl)-6-ethyl-12-methyl-2,5,8,11-tetraoxo-3-phenyl-1,4,7,10-tetraazacyclotetradecan-12-yl)isobutyramide;
N-((3R,6S,9S,12R)-9-(2-((4,5-dihydro-1H-imidazol-2-yl)amino)ethyl)-6-ethyl-12-methyl-2,5,8,11-tetraoxo-3-phenyl-1,4,7,10-tetraazacyclotetradecan-12-yl)isobutyramide;
N-((3R,6S,9S,12R)-9-(2-((1H-imidazol-2-yl)amino)ethyl)-6-ethyl-12-methyl-2,5,8,11-tetraoxo-3-phenyl-1,4,7,10-tetraazacyclotetradecan-12-yl)isobutyramide;
N-((3R,6S,9S,12R)-9-(3-((1H-imidazol-2-yl)amino)propyl)-6-ethyl-12-methyl-2,5,8,11-tetraoxo-3-phenyl-1,4,7,10-tetraazacyclotetradecan-12-yl)isobutyramide;
N-((3R,6S,12R)-6-ethyl-12-methyl-2,5,8,11-tetraoxo-3-phenyl-9-(3-(pyrimidin-2-ylamino)propyl)-1,4,7,10-tetraazacyclotetradecan-12-yl)isobutyramide;
N-((3R,6S,9S,12R)-6-ethyl-12-methyl-2,5,8,11-tetraoxo-3-phenyl-9-(3-((E)-2-(2,2,2-trifluoroethyl)guanidino)propyl)-1,4,7,10-tetraazacyclotetradecan-12-yl)isobutyramide;
N-(3-((5S,8R,13R)-5-ethyl-13-isobutyramido-13-methyl-3,6,9,14-tetraoxo-8-phenyl-1,4,7,10-tetraazacyclotetradecan-2-yl)propyl)acrylamide;
N-(4-((2S,5S,8R,13R)-5-ethyl-13-isobutyramido-13-methyl-3,6,9,14-tetraoxo-8-phenyl-1,4,7,10-tetraazacyclotetradecan-2-yl)butyl)acrylamide;
N-((3R,6S,12R)-9-(3-(2-chloroacetamido)propyl)-6-ethyl-12-methyl-2,5,8,11-tetraoxo-3-phenyl-1,4,7,10-tetraazacyclotetradecan-12-yl)isobutyramide;
N-((3R,6S,9S,12R)-9-(3-(3-acetylguanidino)propyl)-3-(cyclohexylmethyl)-6-ethyl-12-methyl-2,5,8,11-tetraoxo-1,4,7,10-tetraazacyclotetradecan-12-yl)isobutyramide;
N—(N-(3-((2S,5S,8R,13R)-8-(cyclohexylmethyl)-5-ethyl-13-isobutyramido-13-methyl-3,6,9,14-tetraoxo-1,4,7,10-tetraazacyclotetradecan-2-yl)propyl)carbamimidoyl)acrylamide;
N-((3R,6S,9S,12R)-9-(3-(3-(2-chloroacetyl)guanidino)propyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-2,5,8,11-tetraoxo-9-(3-(3-(vinylsulfonyl)guanidino)propyl)-1,4,7,10-tetraazacyclotetradecan-12-yl)isobutyramide;
N-((3R,6S,9S,12R)-3-(cyclohexylmethyl)-9-(3-((4,5-dihydro-1H-imidazol-2-yl)amino)propyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-9-(3-((E)-2,3-dimethylguanidino)propyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-9-(3-(3-nitroguanidino)propyl)-2,5,8,11-tetraoxo-1,4,7,10-tetraazacyclotetradecan-12-yl)isobutyramide;
N-((3R,6S,9S,12R)-9-(3-(3-cyanoguanidino)propyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-9-(3-(hydrazinecarboximidamido)propyl)-12-methyl-2,5,8,11-tetraoxo-1,4,7,10-tetraazacyclotetradecan-12-yl)isobutyramide;
N-((3R,6S,9S,12R)-9-(3-(3-carbamimidoylguanidino)propyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-9-(3-(3,3-dimethylguanidino)propyl)-6-ethyl-12-methyl-2,5,8,11-tetraoxo-1,4,7,10-tetraazacyclotetradecan-12-yl)isobutyramide;
N-((3R,6S,9S,12R)-9-(2-(((1H-imidazol-2-yl)methyl)amino)ethyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-9-(2-(((1H-imidazol-5-yl)methyl)amino)ethyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-9-(3-((1H-pyrazol-4-yl)amino)propyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-2,5,8,11-tetraoxo-9-(3-((1,4,5,6-tetrahydropyrimidin-2-yl)amino)propyl)-1,4,7,10-tetraazacyclotetradecan-12-yl)isobutyramide;
N-((3R,6S,9S,12R)-3-(cyclohexylmethyl)-6-ethyl-12-methyl-9-(2-((1-methylazetidin-3-yl)amino)ethyl)-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-9-(3-((1-methylazetidin-3-yl)amino)propyl)-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-9-(3-(4-methylpiperazin-1-yl)propyl)-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-9-(4-(4-methylpiperazin-1-yl)butyl)-2,5,8,11-tetraoxo-1,4,7,10-tetraazacyclotetradecan-12-yl)isobutyramide;
N-((3R,6S,9S,12R)-3-(cyclohexylmethyl)-6-ethyl-9-(3-(3-ethylguanidino)propyl)-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-2,5,8,11-tetraoxo-9-(3-(3-propylguanidino)propyl)-1,4,7,10-tetraazacyclotetradecan-12-yl)isobutyramide;
N-((3R,6S,9S,12R)-9-(3-(3-butylguanidino)propyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-6-ethyl-9-(3-guanidinopropyl)-3,12-dimethyl-2,5,8,11-tetraoxo-1,4,7,10-tetraazacyclotetradecan-12-yl)isobutyramide;
N-((3R,6S,9S,12R)-6-ethyl-9-(3-guanidinopropyl)-3-isobutyl-12-methyl-2,5,8,11-tetraoxo-1,4,7,10-tetraazacyclotetradecan-12-yl)isobutyramide;
N-((3R,6S,9S,12R)-3-cyclopentyl-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-3-cyclohexyl-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-3-(4-fluorophenyl)-9-(3-guanidinopropyl)-12-methyl-2,5,8,11-tetraoxo-1,4,7,10-tetraazacyclotetradecan-12-yl)isobutyramide;
N-((3R,6S,9S,12R)-3-benzyl-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-3-(2-fluorobenzyl)-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-3-(3-fluorobenzyl)-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-3-(4-fluorobenzyl)-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-9-(3-guanidinopropyl)-12-methyl-3-(naphthalen-1-ylmethyl)-2,5,8,11-tetraoxo-1,4,7,10-tetraazacyclotetradecan-12-yl)isobutyramide;
N-((3R,6S,9S,12R)-3-(cyclopentylmethyl)-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-3-(cyclobutylmethyl)-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-3-(2-cyclohexylethyl)-6-ethyl-9-(3-guanidinopropyl)-12-methyl-2,5,8,11-tetraoxo-1,4,7,10-tetraazacyclotetradecan-12-yl)isobutyramide;
N-((3S,6S,9S,12R)-6-ethyl-12-methyl-9-(3-(3-methylguanidino)propyl)-2,5,8,11-tetraoxo-3-phenyl-1,4,7,10-tetraazacyclotetradecan-12-yl)isobutyramide;
N-((3R,6S,9S,12R)-6-ethyl-9-(3-guanidinopropyl)-3,12-dimethyl-2,5,8,11-tetraoxo-3-phenyl-1,4,7,10-tetraazacyclotetradecan-12-yl)isobutyramide;
N-((3R,6S,9S,12R)-6-ethyl-3-(2-fluorophenyl)-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-3-(3-fluorophenyl)-9-(3-guanidinopropyl)-12-methyl-2,5,8,11-tetraoxo-1,4,7,10-tetraazacyclotetradecan-12-yl)isobutyramide;
N-((3R,6S,9S,12R)-6-ethyl-9-(3-guanidinopropyl)-12-methyl-3-(naphthalen-1-yl)-2,5,8,11-tetraoxo-1,4,7,10-tetraazacyclotetradecan-12-yl)isobutyramide;
N-((3R,6S,9S,12R)-6-ethyl-9-(3-guanidinopropyl)-12-methyl-3-(naphthalen-2-yl)-2,5,8,11-tetraoxo-1,4,7,10-tetraazacyclotetradecan-12-yl)isobutyramide;
N-((3R,6S,9S,12R)-3-(2-chlorophenyl)-6-ethyl-9-(3-guanidinopropyl)-12-methyl-2,5,8,11-tetraoxo-1,4,7,10-tetraazacyclotetradecan-12-yl)isobutyramide;
N-((3R,6S,9S,12R)-3-(3-chlorophenyl)-6-ethyl-9-(3-guanidinopropyl)-12-methyl-2,5,8,11-tetraoxo-1,4,7,10-tetraazacyclotetradecan-12-yl)isobutyramide;
N-((9S,12R,17R)-9-(3-guanidinopropyl)-12-methyl-8,11,16,19-tetraoxo-17-phenyl-7,10,15,18-tetraazaspiro[5.13]nonadecan-12-yl)isobutyramide;
N-((8S,11R,16R)-8-(3-guanidinopropyl)-11-methyl-7,10,15,18-tetraoxo-16-phenyl-6,9,14,17-tetraazaspiro[4.13]octadecan-11-yl)isobutyramide;
N-((3R,6S,9S,12R)-9-(3-((amino(methylamino)methyl)amino)propyl)-3-(cyclohexylmethyl)-6-ethyl-12-methyl-2,5,8,11-tetraoxo-1,4,7,10-tetraazacyclotetradecan-12-yl)isobutyramide;
N-((3R,6S,9S,12R)-9-(3-guanidinopropyl)-12-methyl-2,5,8,11-tetraoxo-3-phenyl-6-propyl-1,4,7,10-tetraazacyclotetradecan-12-yl)isobutyramide;
N-((3R,6S,9S,12R)-9-(3-guanidinopropyl)-6-isopropyl-12-methyl-2,5,8,11-tetraoxo-3-phenyl-1,4,7,10-tetraazacyclotetradecan-12-yl)isobutyramide;
N-((3R,6S,9S,12R)-6-((S)-sec-butyl)-9-(3-guanidinopropyl)-12-methyl-2,5,8,11-tetraoxo-3-phenyl-1,4,7,10-tetraazacyclotetradecan-12-yl)isobutyramide;
N-((3R,6S,9S,12R)-9-(3-(3-acetylguanidino)propyl)-6-ethyl-12-methyl-2,5,8,11-tetraoxo-3-phenyl-1,4,7,10-tetraazacyclotetradecan-12-yl)isobutyramide;
N-((3R,6S,9S,12R)-6-ethyl-12-methyl-2,5,8,11-tetraoxo-3-phenyl-9-(3-((1,4,5,6-tetrahydropyrimidin-2-yl)amino)propyl)-1,4,7,10-tetraazacyclotetradecan-12-yl)isobutyramide;
N-((3R,6S,9S,12R)-6-ethyl-9-(3-guanidinopropyl)-3-(4-methoxyphenyl)-12-methyl-2,5,8,11-tetraoxo-1,4,7,10-tetraazacyclotetradecan-12-yl)isobutyramide;
N-((3R,6S,9S,12R)-6-ethyl-9-(3-guanidinopropyl)-3-(3-methoxyphenyl)-12-methyl-2,5,8,11-tetraoxo-1,4,7,10-tetraazacyclotetradecan-12-yl)isobutyramide;
N-((3R,6S,9S,12R)-3-(cyclohexylmethyl)-9-(3-((5,5-difluoro-1,4,5,6-tetrahydropyrimidin-2-yl)amino)propyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-9-(3-((5,5-dimethyl-1,4,5,6-tetrahydropyrimidin-2-yl)amino)propyl)-6-ethyl-12-methyl-2,5,8,11-tetraoxo-1,4,7,10-tetraazacyclotetradecan-12-yl)isobutyramide;
N-((3R,6S,9S,12R)-3-(cyclohexylmethyl)-6-ethyl-12-methyl-9-(3-((5-methyl-1,4,5,6-tetrahydropyrimidin-2-yl)amino)propyl)-2,5,8,11-tetraoxo-1,4,7,10-tetraazacyclotetradecan-12-yl)isobutyramide;
N-((3R,6S,9S,12R)-6-ethyl-12-methyl-3-(naphthalen-2-yl)-2,5,8,11-tetraoxo-9-(3-((1,4,5,6-tetrahydropyrimidin-2-yl)amino)propyl)-1,4,7,10-tetraazacyclotetradecan-12-yl)isobutyramide;
N-((3R,6S,9S,12R)-6-ethyl-9-(3-guanidinopropyl)-3-(2-methoxyphenyl)-12-methyl-2,5,8,11-tetraoxo-1,4,7,10-tetraazacyclotetradecan-12-yl)isobutyramide; and
N-((3R,6S,9S,12R)-3-(4-chlorophenyl)-6-ethyl-9-(3-guanidinopropyl)-12-methyl-2,5,8,11-tetraoxo-1,4,7,10-tetraazacyclotetradecan-12-yl)isobutyramide.

19. A pharmaceutical composition comprising the compound of claim 1 or a pharmaceutically acceptable salt or hydrate thereof, and a pharmaceutically acceptable carrier.

20. A method of treating a patient, the method comprising administering to the patient a therapeutically effective amount of the compound of claim 1, or a pharmaceutically acceptable salt or solvate thereof, wherein the patient has cancer, a chronic autoimmune disorder, an inflammatory condition, or a proliferative disorder.

21. The method claim 20, wherein the patient has cancer.

22. The method of claim 21, wherein the cancer is selected from any one or more of the cancers of Table 2.

23. The method of claim 21, wherein the cancer is leukemia.

24. The method of claim 20 further comprising administering a therapeutically effective amount of a second therapeutic agent useful in the treatment of the disease or condition.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)
